SEQUENCE LISTING (81 sequences)

SEQ ID NO: 1 - protein, 4588 aa, homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGGIDWDSHPFGYNLTLQAKDKGTPPQFSSVKVIHVTSPQFKAGPVKFEKDVYRAEISEFAPPNTPVVMVKAIPAYSHLRYVFKSTPGKAKFSLNYNTGLISILEPVKRQQAAHFELEVTTSDRKASTKVLVKVLGANSNPPEFTQTAYKAAFDENVPIGTTVMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDFFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSIVTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDARRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYHVIATDKDEGPNAEISYSIEDGNEHGKFFIEPKTGVVSSKRFSAAGEYDILSIKAVDNGRPQKSSTTRLHIEWISKPKPSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVHQHTLTVMVRDQDVPVKRNFARIVVNVSDTNDHAPWFTASSYKGRVYESAAVGSVVLQVTALDKDKGKNAEVLYSIESGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGNPINEPLFYHILNPDRRFKISRTSGVLSTTGTPFDREQQEAFDVVVEVTEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNDNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKVLASDADEGSNADITYAIEADSESVKENLEINKLSGVITTKESLIGLENEFFTFFVRAVDNGSPSKESVVLVYVKILPPEMQLPKFSEPFYTFTVSEDVPIGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVLTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSKPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV

SEQ ID NO: 2 - protein, 21 aa, homo sapiens
MGRHLALLLLLLLLFQHFGDS

SEQ ID NO: 3 - DNA, 11599 bp, homo sapiens
gagcgtgaggcgccggcgccgagctgggcggccgggcgcggggagagggcgcgggagcggctcgtgcggcaggtaccatgcggacgcgcgagcccggcgagggccccggcaggcccggtccctgctcgggggcgcgctgagacggcgggtgagctccacgagagcgccgtcgccacttcgggccaactttgcgattcccgacagttaagcaatggggagacatttggctttgctcctgcttctgctccttctcttccaacattttggagacagtgatggcagccaacgacttgaacagactcctctgcagtttacacacctcgagtacaacgtcaccgtgcaggagaactctgcagctaagacttatgtggggcatcctgtcaagatgggtgtttacattacacatccagcgtgggaagtaaggtacaaaattgtttccggagacagtgaaaacctgttcaaagctgaagagtacattctcggagacttttgctttctaagaataaggaccaaaggaggaaatacagctattcttaatagagaagtgaaggatcactacacattgatagtgaaagcacttgaaaaaaatactaatgtggaggcgcgaacaaaggtcagggtgcaggtgctggatacaaatgacttgagaccgttattctcacccacctcatacagcgtttctttacctgaaaacacagctataaggaccagtatcgcaagagtcagcgccacggatgcagacataggaaccaacggggaattttactacagttttaaagatcgaacagatatgtttgctattcacccaaccagtggtgtgatagtgttaactggtagacttgattacctagagaccaagctctatgagatggaaatcctcgctgcggaccgtggcatgaagttgtatgggagcagtggcatcagcagcatggccaagctaacggtgcacatcgaacaggccaatgaatgtgctccggtgataacagcagtgacattgtcaccatcagaactggacagggacccagcatatgcaattgtgacagtggatgactgcgatcagggtgccaatggtgacatagcatctttaagcatcgtggcaggtgaccttctccagcagtttagaacagtgaggtcctttccagggagtaaggagtataaagtcaaagccatcggtggcattgattgggacagtcatcctttcggctacaatctcacactacaggctaaagataaaggaactccgccccagttctcttctgttaaagtcattcacgtgacttctccacagttcaaagccgggccagtcaagtttgaaaaggatgtttacagagcagaaataagtgaatttgctcctcccaacacacctgtggtcatggtaaaggccattcctgcttattcccatttgaggtatgtttttaaaagtacacctggaaaagctaaattcagtttaaattacaacactggtctcatttctattttagaaccagttaaaagacagcaggcagcccattttgaacttgaagtaacaacaagtgacagaaaagcgtccaccaaggtcttggtgaaagtcttaggtgcaaatagcaatccccctgaatttacccagacagcgtacaaagctgcttttgatgagaacgtgcccattggtactactgtcatgagcctgagtgccgtagaccctgatgagggtgagaacgggtacgtgacatacagtatcgcaaatttaaatcatgtgccgtttgcgattgaccatttcactggtgccgtgagtacgtcagaaaacctggactacgaactgatgcctcgggtttatactctgaggattcgtgcatcagactggggcttgccgtaccgccgggaagtcgaagtccttgctacaattactctcaataacttgaatgacaacacacctttgtttgagaaaataaattgtgaagggacaattcccagagatctaggcgtgggagagcaaataaccactgtttctgctattgatgcagatgaacttcagttggtacagtatcagattgaagctggaaatgaactggatttctttagtttaaaccccaactcgggggtattgtcattaaagcgatcgctaatggatggcttaggtgcaaaggtgtctttccacagtctgagaatcacagctacagatggagaaaattttgccacaccattatatatcaacataacagtggctgccagtcacaagctggtaaacttgcagtgtgaagagactggtgttgccaaaatgctggcagagaagctcctgcaggcaaataaattacacaaccagggagaggtggaggatattttcttcgattctcactctgtcaatgctcacataccgcagtttagaagcactcttccgactggtattcaggtaaaggaaaaccagcctgtgggttccagtgtaattttcatgaactccactgaccttgacactggcttcaatggaaaactggtctatgctgtttctggaggaaatgaggatagttgcttcatgattgatatggaaacaggaatgctgaaaattttatctcctcttgaccgtgaaacaacagacaaatacaccctgaatattaccgtctatgaccttgggataccccagaaggctgcgtggcgtcttctacatgtcgtggttgtcgatgccaatgataatccacccgagtttttacaggagagctattttgtggaagtgagtgaagacaaggaggtacatagtgaaatcatccaggttgaagccacagataaagacctggggcccaacggacacgtgacgtactcaattgttacagacacagacacattttcaattgacagcgtgacgggtgttgttaacatcgcacgccctctggatcgagagctgcagcatgagcactccttaaagattgaggccagggaccaagccagagaagagcctcagctgttctccactgtcgttgtgaaagtatcactagaagatgttaatgacaacccacctacatttattccacctaattatcgtgtgaaagtccgagaggatcttccagaaggaaccgtcatcatgtggttagaagcccacgatcctgatttaggtcagtctggtcaggtgagatacagccttctggaccacggagaaggaaacttcgatgtggataaactcagtggagcagttaggatcgtccagcagttggactttgagaagaagcaagtgtataatctcactgtgagggccaaagacaagggaaagccagtttctctgtcttctacttgctatgttgaagttgaggtggttgatgtgaatgagaacctgcacccacccgtgttttccagctttgtggaaaaggggacagtgaaagaagatgcacctgttggttcattggtaatgacggtgtcggctcatgatgaggacgccagaagagatggggagatccgatactccattagagatggctctggcgttggtgttttcaaaataggtgaagagacaggtgtcatagagacgtcagatcgactggaccgtgaatcgacctcccattattggctaacagtctttgcaaccgatcagggtgtcgtgcctctttcatcgttcatagagatctacatagaggttgaggatgtcaatgacaatgcaccacagacatcagagcctgtttattacccagaaatcatggaaaattctcctaaagatgtatctgtggtccagatcgaggcatttgatccagattcgagctctaatgacaagctcatgtacaaaattacaagtggaaatccacaaggattcttttcaatacatcctaaaacaggtctcatcacaactacgtcaaggaagctagaccgagaacagcaagatgaacacatattagaggttactgtgacagacaatggtagtccccccaaatcaaccattgcaagagtcattgtgaaaatccttgatgaaaatgacaacaaacctcagtttctgcaaaagttctacaaaatcagactccctgagcgggaaaagccagaccgagaaagaaatgccagacgggagccgctctatcacgtcatagccaccgacaaggatgagggccccaatgcagaaatctcctacagcatcgaagacgggaatgagcatggcaaatttttcatcgaaccgaaaactggagtggtttcgtccaagaggttttcagcagctggagaatatgatattctttcaattaaggcagttgacaatggtcgccctcaaaagtcatcaaccaccagactccatattgaatggatctccaagcccaaaccgtccctggagcccatttcatttgaagaatcattttttacctttactgtgatggaaagtgaccccgttgctcacatgattggagtaatatctgtggagcctcctggcatacccctttggtttgacatcactggtggcaactacgacagtcacttcgatgtggacaagggaactggaaccatcattgttgccaaacctcttgatgcagaacagaagtcaaactacaacctcacagtcgaggctacagatggaaccaccactatcctcactcaggtattcatcaaagtaatagacacaaatgaccatcgtcctcagttttctacatcaaagtatgaagttgttattcctgaagatacagcgccagaaacagaaattttgcaaatcagtgctgtggatcaggatgagaaaaacaaactaatctacactctgcagagcagtagagatccactgagtctcaagaaatttcgtcttgatcctgcaaccggctctctctatacttctgagaaactggatcatgaagctgttcaccagcacaccctcacggtcatggtacgagatcaagatgtgcctgtaaaacgcaactttgcaaggattgtggtcaatgtcagcgacacgaatgaccacgccccgtggttcaccgcttcctcctacaaagggcgggtttatgaatcggcagccgttggctcagttgtgttgcaggtgacggctctggacaaggacaaagggaaaaatgctgaagtgctgtactcgatcgagtcaggaaatattggaaattcttttatgattgatcctgtcttgggctctattaaaactgccaaagaattagatcgaagtaaccaagcggagtatgatttaatggtaaaagctacagataagggcagtccaccaatgagtgaaataacttctgtgcgtatctttgtcacaattgctgacaacgcctctccgaagtttacatcaaaagaatattctgttgaacttagtgaaactgtcagcattgggagtttcgttgggatggttacagcccatagtcaatcatcagtggtgtatgaaataaaagatggaaatacaggtgatgcttttgatattaatccacattctggaactatcatcactcagaaagccctggactttgaaactttgcccatttacacattgataatacaaggaactaacatggctggtttgtccactaatacaacggttctagttcacttgcaggatgagaatgacaacgcgccagtttttatgcaggcagaatatacaggactcattagtgaatcagcctcaattaacagcgtggtcctaacagacaggaatgtcccactggtgattcgagcagctgatgctgataaagactcaaatgctttgcttgtatatcacattgttgaaccatctgtacacacatattttgctattgattctagcactggtgctattcatacagtactaagtctggactatgaagaaacaagtatttttcactttaccgtccaagtgcatgacatgggaaccccacgtttatttgctgagtatgcagcgaatgtaacagtacatgtaattgacattaatgactgcccccctgtgtttgccaagccattatatgaagcatctcttttgttaccaacatacaaaggagtaaaagtcatcacagtaaatgctacagatgctgattcaagtgcattctcacagttgatttactccatcaccgaaggcaacatcggggagaagttttctatggactacaagactggtgctctcactgtccaaaacacaactcagttaagaagccgctacgagctaaccgttagagcttccgatggcagatttgccggccttacctctgtcaaaattaatgtgaaagaaagcaaagaaagtcacctaaagtttacccaggatgtctactctgcggtagtgaaagagaattccaccgaggccgaaacattagctgtcattactgctattgggaatccaatcaatgagcctttgttttatcacatcctcaacccagatcgcagatttaaaataagccgcacttcaggagttctgtcaaccactggcacgcccttcgatcgtgagcagcaggaggcgtttgatgtggttgtagaagtgacagaggaacataagccttctgcagtggcccacgttgtcgtgaaggtcattgtagaagaccaaaatgataatgcgccggtgtttgtcaaccttccctactacgccgttgttaaagtggacactgaggtgggccatgtcattcgctatgtcactgctgtagacagagacagtggcagaaacggggaagtgcattactacctcaaggaacatcatgaacactttcaaattggacccttgggtgaaatttcactgaaaaagcaatttgagcttgacaccttaaataaagaatatcttgttacagtggttgcaaaagatggagggaacccggccttttcagcggaagttatcgttccgatcactgtcatgaataaagccatgcctgtgtttgaaaaacctttctacagtgcagagattgcagagagcatccaggtgcacagccctgtggtccacgtgcaggctaacagcccggaaggcctgaaagtgttctacagcatcacagacggagaccctttcagccagttcactattaacttcaatactggagttatcaatgtcatagctcctctggactttgaggcccacccggcatataagctgagcatacgcgcaactgactccttgacgggcgctcatgctgaagtatttgtggacatcatagtagacgacatcaatgataaccctcctgtgtttgctcagcagtcttatgcggtgaccctgtctgaggcatctgtaattggaacgtctgttgttcaagttagagccaccgattctgattcagaaccaaatagaggaatctcataccagatgtttgggaatcacagcaagagtcatgatcattttcatgtagacagcagcactggcctcatctcactactcagaaccctggattacgagcagtcccggcagcacacgatttttgtgagggcagttgatggtggtatgcccacgctgagcagtgatgtgattgtcacggtggacgttaccgacctcaatgataatccaccactctttgaacaacagatttatgaagccagaattagcgagcacgcccctcatgggcatttcgtgacctgtgtaaaagcctatgatgcagacagttcagacatagacaagttgcagtattccattctgtctggcaatgatcataaacattttgtcattgacagtgcaacagggattatcaccctctcaaacctgcaccggcacgccctgaagccattttacagtcttaacctgtcagtgtctgatggagtttttagaagttccacccaggttcatgtaactgtaattggaggcaatttgcacagtcctgctttccttcagaacgaatatgaagtggaactagctgaaaacgctcccctacataccctggtgatggaggtgaaaactacggatggggattctggtatttatggtcacgttacttaccatattgtaaatgactttgccaaagacagattttacataaatgagagaggacagatatttactttggaaaaacttgatcgagaaaccccggcggagaaagtgatctcagtccgtttaatggctaaggatgctggaggaaaagttgctttctgcaccgtgaatgtcatccttacagatgacaatgacaatgcaccacaatttcgagcaaccaaatacgaagtgaatatcgggtccagtgctgctaaagggacttcagtcgttaaagttcttgcaagtgatgccgatgagggctccaatgccgacatcacctatgccattgaagcagactctgaaagtgtaaaagagaatttggaaattaacaaactgtccggcgtaatcactacaaaggagagcctcattggcttggaaaatgaattcttcactttctttgttagagctgtggataatgggtctccatcaaaagaatctgttgttcttgtctatgttaaaatccttccaccggaaatgcagcttccaaaattttcagaacctttctatacctttacagtgtcagaggacgtgcctattggaacagagatagatctcatccgagcagaacatagtgggactgttctttacagcctggtcaaagggaatactccagaaagcaatagggatgagtcctttgtgattgacagacagagcgggagactgaagttggagaagagtcttgatcatgagacaactaagtggtatcagttttccatactggccaggtgcactcaagatgaccatgagatggtggcttctgtagatgttagtatccaagtgaaagatgcaaatgacaacagcccggtctttgaatctagtccatatgaggcattcattgttgaaaacctgccagggggaagtagagtaattcagatcagggcatctgatgctgactcaggaaccaacggccaagttatgtatagcctggatcagtcacaaagtgtggaagtcattgaatcctttgccattaacatggaaacaggctggattacaactttaaaggaacttgaccatgaaaagagagacaattaccagattaaagtggttgcatcagatcatggtgaaaagatccagctatcctccacagccattgtggatgttaccgtcaccgatgtcaacgatagtccaccacgattcacggccgagatctataaagggactgtgagtgaggatgacccccaaggtggggtgattgccatcttaagtaccacggatgctgattctgaagagatcaacagacaagttacatatttcataacaggaggggatcctttaggacagtttgccgttgaaactatacagaatgaatggaaggtatatgtgaagaaacctctagacagggaaaaaagggacaattaccttcttactatcacggcaactgatggcaccttctcatcaaaagcgatagttgaagtgaaagttctggatgcaaatgacaacagtccagtttgtgaaaagactttatattcagacactattcctgaagacgtccttcctggaaaattgatcatgcagatctctgctacagacgcagacatccgctctaacgctgaaattacttacacgttattgggttcaggtgcagaaaaattcaaactaaatccagacacaggtgaactgaaaacgtcaaccccccttgatcgtgaggagcaagctgtttatcatcttctcgtcagggccacagatggaggaggaagattctgccaagccagtattgtgctcacgctagaagatgtgaacgataacgcccccgaattctctgccgatccttatgccatcaccgtgtttgaaaacacagagccgggaacgctgctgacaagagtgcaggccacagatgccgacgcaggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcagttcttgacataaatgacaacccccctgtgtttgagtaccgtgaatatggtgccaccgtgtctgaggacattcttgttggaactgaagttcttcaagtgtatgcagcaagtcgggatattgaagcaaatgcagaaatcacctactcaataataagtggaaatgaacatgggaaattcagcatagattctaaaacaggggccgtatttatcattgagaatctggattatgagagctctcatgagtattacctaacagtagaggccactgatggaggcacgccttcactgagcgacgttgccactgtgaacgttaatgtaacagatatcaacgataatacccctgtgttcagccaagacacctacacgacagtcatcagtgaagatgccgttcttgagcagtctgtcatcacggttatggccgatgatgccgatggaccttccaacagccacatccactactcaattatagatggcaaccaaggaagctcgttcacaattgaccccgtcaggggagaagtcaaagtgaccaaacttctcgaccgagaaacgatttcaggttacacgctcacggttcaagcttctgataatggcagtccacccagagtcaacacgacgaccgtgaacatcgatgtgtccgatgtcaatgacaacgcgcccgtcttctccaggggaaactacagtgtcattatccaggaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtggcagataatggaaagcctcagttgtcatctttgacatacattgacattagggtaattgaggagagcatctatccgcctgcgattttgcccctggagattttcatcacctcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaagagggaaggtgcccacctgtccaccatggctgtgaagat

SEQ ID NO: 4 - protein, 7 aa, artificial sequence
  protein / consensus sequence
SSRCGCC

SEQ ID NO: 5 - protein, 7 aa, artificial sequence
  protein / consensus sequence
RTDKKKK

SEQ ID NO: 6 - DNA, 10 bp, artificial sequence
  nucleic acid / consensus sequence
  misc_feature (1)..(1): N can be C, G, T or A
  misc_feature (2)..(2): N can be C, T or G
  misc_feature (3)..(3): N can be C or T
  misc_feature (4)..(4): N can be A or T
  misc_feature (9)..(9): N can be T or C
  misc_feature (10)..(10): N can be C, T, A or G
nnnnttgtnn

SEQ ID NO: 7 - DNA, 10 bp, artificial sequence
  nucleic acid / consensus sequence
  misc_feature (1)..(3): N can be C, G, A or T
  misc_feature (4)..(4): N can be G or C
  misc_feature (6)..(6): N can be A, T, or C
  misc_feature (9)..(9): N can be G, T, or C
  misc_feature (10)..(10): N can be C, T or A
nnnngnatnn

SEQ ID NO: 8 - DNA, 25 bp, artificial sequence
  hFat1_L1_exon2 +
caccgagtggctgccagtcacaagc

SEQ ID NO: 9 - DNA, 25 bp, artificial sequence
  hFat1_L1_exon2 -
aaacgcttgtgactggcagccactc

SEQ ID NO: 10 - DNA, 25 bp, artificial sequence
  hFat1_L2_exon2 +
caccgtatgttgatatataatggtg

SEQ ID NO: 11 - DNA, 25 bp, artificial sequence
  hFat1_L2_exon2 -
aaaccaccattatatatcaacatac

SEQ ID NO: 12 - DNA, 25 bp, artificial sequence
  hFat1_R1_exon2 +
caccgactggggcttgccgtaccgc

SEQ ID NO: 13 - DNA, 25 bp, artificial sequence
  hFat1_R1_exon2 -
aaacgcggtacggcaagccccagtc

SEQ ID NO: 14 - DNA, 25 bp, artificial sequence
  hFat1_R2_exon2 +
caccgatcctcagagtataaacccg

SEQ ID NO: 15 - DNA, 25 bp, artificial sequence
  hFat1_R2_exon2 -
aaaccgggtttatactctgaggatc

SEQ ID NO: 16 - DNA, 25 bp, artificial sequence
  hCD44 del sg1For
caccgaagactcccattcgacaaca

SEQ ID NO: 17 - DNA, 25 bp, artificial sequence
  hCD44 del sg1Rev
aaactgttgtcgaatgggagtcttc

SEQ ID NO: 18 - DNA, 25 bp, artificial sequence
  hCD44 del sg2For
caccgcatcacggttaacaatagct

SEQ ID NO: 19 - DNA, 25 bp, artificial sequence
  hCD44 del sg2Rev
aaacagctattgttaaccgtgatgc

SEQ ID NO: 20 - DNA, 25 bp, artificial sequence
  hCD44 del sg3For
caccgtcgctacagcatctctcgga

SEQ ID NO: 21 - DNA, 25 bp, artificial sequence
  hCD44 del sg3Rev
aaactccgagagatgctgtagcgac

SEQ ID NO: 22 - DNA, 25 bp, artificial sequence
  hCD44 del sg4For
caccgtgctacttcagacaaccaca

SEQ ID NO: 23 - DNA, 25 bp, artificial sequence
  hCD44 del sg4Rev
aaactgtggttgtctgaagtagcac

SEQ ID NO: 24 - DNA, 25 bp, artificial sequence
  hYAP1 del sg1For
caccggatgaacctttaccaaaacg

SEQ ID NO: 25 - DNA, 25 bp, artificial sequence
  hYAP1 del sg1Rev
aaaccgttttggtaaaggttcatcc

SEQ ID NO: 26 - DNA, 25 bp, artificial sequence
  hYAP1 del sg2For
caccggtgcacgatctgatgcccgg

SEQ ID NO: 27 - DNA, 25 bp, artificial sequence
  hYAP1del sg2Rev
aaacccgggcatcagatcgtgcacc

SEQ ID NO: 28 - DNA, 25 bp, artificial sequence
  hYAP1 del sg3For
caccgtcgaacatgctgtggagtca

SEQ ID NO: 29 - DNA, 25 bp, artificial sequence
  hYAP1 del sg3Rev
aaactgactccacagcatgttcgac

SEQ ID NO: 30 - DNA, 25 bp, artificial sequence
  hYAP1 del sg4For
caccgtgccccagaccgtgcccatg

SEQ ID NO: 31 - DNA, 25 bp, artificial sequence
  hYAP1 del sg4Rev
aaaccatgggcacggtctggggcac

SEQ ID NO: 32 - DNA, 25 bp, artificial sequence
  hSOX2 del sg1For
caccgattataaataccggccccgg

SEQ ID NO: 33 - DNA, 25 bp, artificial sequence
  hSOX2 del sg1Rev
aaacccggggccggtatttataatc

SEQ ID NO: 34 - DNA, 25 bp, artificial sequence
  hSOX2 del sg2For
caccgcgttcatcgacgaggctaag

SEQ ID NO: 35 - DNA, 25 bp, artificial sequence
  hSOX2del sg2Rev
aaaccttagcctcgtcgatgaacgc

SEQ ID NO: 36 - DNA, 25 bp, artificial sequence
  hSOX2 del sg3For
caccggacagttacgcgcacatgaa

SEQ ID NO: 37 - DNA, 25 bp, artificial sequence
  hSOX2 del sg3Rev
aaacttcatgtgcgcgtaactgtcc

SEQ ID NO: 38 - DNA, 25 bp, artificial sequence
  hSOX2 del sg4For
caccggcagggcgctcacgtcgtag

SEQ ID NO: 39 - DNA, 25 bp, artificial sequence
  hSOX2 del sg4Rev
aaacctacgacgtgagcgccctgcc

SEQ ID NO: 40 - DNA, 25 bp, artificial sequence
  hCDH1 del sg1For
caccgaagtcacgctgaatacagtg

SEQ ID NO: 41 - DNA, 25 bp, artificial sequence
  hCDH1 del sg1Rev
aaaccactgtattcagcgtgacttc

SEQ ID NO: 42 - DNA, 25 bp, artificial sequence
  hCDH1 del sg2For
caccgctggagattaatccggacac

SEQ ID NO: 43 - DNA, 25 bp, artificial sequence
  hCDH1 del sg2Rev
aaacgtgtccggattaatctccagc

SEQ ID NO: 44 - DNA, 25 bp, artificial sequence
  hCDH1 del sg3For
caccggcaatgcgttctctatccag

SEQ ID NO: 45 - DNA, 25 bp, artificial sequence
  hCDH1 del sg3Rev
aaacctggatagagaacgcattgcc

SEQ ID NO: 46 - DNA, 25 bp, artificial sequence
  hCDH1 del sg4For
caccgtgaaccaccagggtatacgt

SEQ ID NO: 47 - DNA, 25 bp, artificial sequence
  hCDH1 del sg4Rev
aaacacgtataccctggtggttcac

SEQ ID NO: 48 - DNA, 25 bp, artificial sequence
  mYap1 sg1Rev
aaaccggggactcggagaccgactc

SEQ ID NO: 49 - DNA, 25 bp, artificial sequence
  mYap1 sg2For
caccgtgccgtcatgaaccccaaga

SEQ ID NO: 50 - DNA, 25 bp, artificial sequence
  mYap1 sg2Rev
aaactcttggggttcatgacggcac

SEQ ID NO: 51 - DNA, 25 bp, artificial sequence
  mYap1 sg3For
caccgggcggcttgaagaaggagtc

SEQ ID NO: 52 - DNA, 25 bp, artificial sequence
  mYap1 sg3Rev
aaacgactccttcttcaagccgccc

SEQ ID NO: 53 - DNA, 25 bp, artificial sequence
  mYap1 sg4For
caccgcgacaggtaagggtatcccg

SEQ ID NO: 54 - DNA, 25 bp, artificial sequence
  mYap1 sg4Rev
aaaccgggatacccttacctgtcgc

SEQ ID NO: 55 - DNA, 25 bp, artificial sequence
  mSox2 sg1For
caccgaggaggcaacgccacggcgg

SEQ ID NO: 56 - DNA, 25 bp, artificial sequence
  mSox2 sg1Rev
aaacccgccgtggcgttgcctcctc

SEQ ID NO: 57 - DNA, 25 bp, artificial sequence
  mSox2 sg2For
caccgtgggcctcttgacgcggtcc

SEQ ID NO: 58 - DNA, 25 bp, artificial sequence
  mSox2 sg2Rev
aaacggaccgcgtcaagaggcccac

SEQ ID NO: 59 - DNA, 25 bp, artificial sequence
  mSox2 sg3For
caccgggtgggcgagccgttcatgt

SEQ ID NO: 60 - DNA, 25 bp, artificial sequence
  mSox2 sg3Rev
aaacacatgaacggctcgcccaccc

SEQ ID NO: 61 - DNA, 25 bp, artificial sequence
  mSox2 sg4For
caccggggcacccccggtatggcgc

SEQ ID NO: 62 - DNA, 25 bp, artificial sequence
  mSox2 sg4Rev
aaacgcgccataccgggggtgcccc

SEQ ID NO: 63 - DNA, 25 bp, artificial sequence
  mCdh1_sg1For
caccgggagaacgaggaaccctttg

SEQ ID NO: 64 - DNA, 25 bp, artificial sequence
  mCdh1_sg1Rev
aaaccaaagggttcctcgttctccc

SEQ ID NO: 65 - DNA, 25 bp, artificial sequence
  mCdh1_sg2For
caccggtccacagtgacagtggctg

SEQ ID NO: 66 - DNA, 25 bp, artificial sequence
  mCdh1_sg2Rev
aaaccagccactgtcactgtggacc

SEQ ID NO: 67 - DNA, 25 bp, artificial sequence
  mCdh1_sg3For
caccggtcgaagtgcccgaagactt

SEQ ID NO: 68 - DNA, 25 bp, artificial sequence
  mCdh1_sg3Rev
aaacaagtcttcgggcacttcgacc

SEQ ID NO: 69 - DNA, 25 bp, artificial sequence
  mCdh1_sg4For
caccggaacgtgtccggctctcgag

SEQ ID NO: 70 - DNA, 25 bp, artificial sequence
  mCdh1_sg4Rev
aaacctcgagagccggacacgttcc

SEQ ID NO: 71 - DNA, 25 bp, artificial sequence
  mTAZ del sg1For
caccgacatagagaaaatcaccaca

SEQ ID NO: 72 - DNA, 25 bp, artificial sequence
  mTAZ del sg1Rev
aaactgtggtgattttctctatgtc

SEQ ID NO: 73 - DNA, 25 bp, artificial sequence
  mTAZ del sg2For
caccggcaagtcatccacgtcacgc

SEQ ID NO: 74 - DNA, 25 bp, artificial sequence
  mTAZ del sg2Rev
aaacgcgtgacgtggatgacttgcc

SEQ ID NO: 75 - DNA, 25 bp, artificial sequence
  mTAZ del sg3For
caccgtgggttggttctgagtcggg

SEQ ID NO: 76 - DNA, 25 bp, artificial sequence
  mTAZ4 del sg3Rev
aaaccccgactcagaaccaacccac

SEQ ID NO: 77 - DNA, 25 bp, artificial sequence
  mTAZ del sg4For
caccggaggattaggatgcgtcaag

SEQ ID NO: 78 - DNA, 25 bp, artificial sequence
  mTAZ del sg4Rev
aaaccttgacgcatcctaatcctcc

SEQ ID NO: 79 - DNA, 21 bp, artificial sequence
  Fat1_exon2_external_Forward
gcggtatgtgagcattgacag

SEQ ID NO: 80 - DNA, 20 bp, artificial sequence
  Fat1_exon2_internal_Reverse
cccaactcgggggtattgtc

SEQ ID NO: 81 - DNA, 19 bp, artificial sequence
  Fat1_exon2_external_Reverse
gggtgagaacgggtacgtg

(19) United States
(12) Patent Application Publication
(10) Pub. No.: US 2024/0026458 A1
(43) Pub. Date: Jan. 25, 2024

(54) Method for Determining Sensitivity to an Antineoplastic Agent

(71) Applicant: UNIVERSITÉ LIBRE DE BRUXELLES, Brussels (BE)

(72) Inventors: Cédric Blanpain, Ohain (BE); Ievgenia Pastushenko, Uccle (BE); Frederico Mauri, Vienna (AT)

(21) Appl. No.: 18/015,901

(22) PCT Filed: Jul. 15, 2021
(86) PCT No.: PCT/EP2021/069685

§ 371 Date: Jan. 12, 2023

(30) Foreign Application Priority Data
Jul. 15, 2020 (EP) 20185880.0

(51) Int. Cl. C12Q 1/6886; G01N 33/574; C12Q 1/6851; C12Q 1/6869

(52) U.S. Cl. CPC C12Q 1/6886; G01N 33/57492; C12Q 1/6851; C12Q 1/6869; C12Q 2600/156; C12Q 2600/158; C12Q 2600/106; G01N 2440/14

(57) ABSTRACT

Present invention provides methods for determining sensitivity or resistance to treatment with an antineoplastic agent in a subject diagnosed with a neoplastic disease, the method comprising determining in a biological sample obtained from said subject the presence or absence of a genetic or epigenetic alteration leading to reduced or abolished expression or function of FAT1, or determining whether FAT1 expression or function is reduced or abolished in a biological sample obtained from said subject; wherein said antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor. Present invention further also provides methods of treating a subject diagnosed with a neoplastic disease, comprising determining the sensitivity or resistance of said subject to treatment with an antineoplastic agent.

### CROSS-REFERENCE TO RELATED APPLICATIONS

This application is a national phase entry under 35 U.S.C. § 371 of International Patent Application PCT/EP2021/069685, filed Jul. 15, 2021, designating the United States of America and published in English as International Patent Publication WO 2022/013328 on Jan. 20, 2022, which claims the benefit under Article 8 of the Patent Cooperation Treaty to European Patent Application Serial No. 20185880.0, filed Jul. 15, 2020, the entireties of which are hereby incorporated by reference.

### FIELD OF THE INVENTION

The present invention is broadly in the therapeutic and companion diagnostic fields. The invention particularly relates to methods for determining sensitivity of a subject diagnosed with a neoplastic disease to an antineoplastic agent, in particular an antineoplastic agent selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor and a SRC kinase inhibitor. The present invention further relates to treating those patients who are determined to be sensitive to said antineoplastic agent with said antineoplastic agent.

### BACKGROUND OF THE INVENTION

The sensitivity of neoplastic diseases to antineoplastic agents may vary and can depend to some extent on their molecular subtype. A companion diagnostic for an antineoplastic agent is therefore valuable to determine whether a patient will benefit from a treatment with the antineoplastic agent. In particular, gene mutations, epigenetic alterations and chromosomal rearrangements have been found to play a functional role in tumor responses to antineoplastic agents.

FAT1 is very frequently mutated in a broad range of human cancers, in particular in squamous cell carcinomas (SCC) originating from various body locations including skin, head and neck, oesophagus and lung. In the most frequently used cancer mouse model, skin SCC induced by the chemical carcinogen (7,12-dimethylbenzanthracene (DMBA)/12-O-tetradecanoylphorbol-13-acetate (TPA)), Fat1 is mutated in about 20% of the cases, as in human SCC. Stop-gain mutations (mutations that result in a premature translation termination codon) are very frequently found, indicating that these mutations most likely result in loss of function (LOF) and that FAT1 acts as a tumor suppressor gene (TSG). Short hairpin RNA (shRNA) mediated knock-down of FAT1 in human cancer cell lines decreased cell-cell adhesion and promoted cell migration, whereas contradictory results were obtained on the role of FAT1 in regulating Epithelial to Mesenchymal Transition (EMT) in cancer cell lines in vitro. However, the molecular mechanisms by which FAT1 mutation promotes tumorigenesis and controls tumor heterogeneity in vivo are completely unknown.

The treatment of advanced squamous cell carcinomas (SCCs) from different organs, such as head and neck, oesophagus or lung, remains challenging, and the prognosis is particularly poor in metastatic disease. There thus remains a need in the art to improve the assessment of therapeutic sensitivity of neoplastic diseases, and in particular SCC, to neoplastic agents.

### SUMMARY OF THE INVENTION

The present invention is at least in part based on the inventors' insight that certain signalling cascades, such as those including epidermal growth factor receptor (EGFR), a mitogen-activated protein kinase (MEK), a Ca²⁺/calmodulin-dependent protein kinase (CAMK) or a SRC kinase, are impacted when FAT1 function is perturbed or abolished. Furthermore, the present inventors found that because the aforementioned signaling cascades can be targeted by antineoplastic agents, the differential activation of the signaling cascades in neoplastic diseases which involve reduced or abolished expression or function of FAT1 allows to predict therapeutic resistance or sensitivity of subjects to such antineoplastic agents based on the FAT1 status of the subjects' neoplastic diseases.

Accordingly, an aspect provides a method for determining sensitivity or resistance to treatment with an antineoplastic agent in a subject diagnosed with a neoplastic disease, the method comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject;
   wherein said antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor.

A further aspect provides a method of treating a subject diagnosed with a neoplastic disease, comprising
determining the sensitivity or resistance of said subject to treatment with an antineoplastic agent comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject,
wherein said antineoplastic agent is selected from the group consisting of an EGFR inhibitor, a MEK inhibitor, a CAMK inhibitor, and a SRC kinase inhibitor; and
treating said subject with said antineoplastic agent if said subject is determined to be sensitive to said antineoplastic agent.

A further aspect provides a kit for determining sensitivity or resistance to treatment with an antineoplastic agent, comprising
means for determining in a biological sample the presence or absence of a genetic or epigenetic alteration leading to reduced or abolished expression or function of FAT1, and/or
means for determining FAT1 expression or function in a biological sample,
   and a computer readable storage medium having recorded thereon one or more programs for carrying out the method as taught herein.

A further aspect provides an antineoplastic agent selected from the group consisting of a CAMK inhibitor, a SRC kinase inhibitor, and a combination thereof, for use in the treatment of a neoplastic disease in a subject, wherein said subject has been selected as having a neoplastic disease characterized by
a genetic or epigenetic alteration leading to reduced or abolished expression or function of FAT1, or
reduced or abolished FAT1 expression or function.

Another aspect provides an antineoplastic agent selected from the group consisting of an EGFR inhibitor, a MEK inhibitor, and a combination thereof, for use in the treatment of a neoplastic disease in a subject, wherein said subject has been selected as having a neoplastic disease characterized by
the absence of a genetic or epigenetic alteration leading to reduced or abolished expression or function of FAT1, or
normal or increased FAT1 expression or function.

These and further aspects and preferred embodiments of the invention are described in the following sections and in the appended claims. The subject-matter of the appended claims is hereby specifically incorporated in this specification.

### DESCRIPTION OF THE DRAWINGS

FIGS. 1A-1F Nucleic acid sequence of human FAT1 mRNA. The coding sequence is underlined and in bold. The sequence encoding the signal peptide is italicized.

FIG. 2 FIG. 2A, Box Plot showing the distribution of the common mRNA signature (mouse skin and lung Fat1 conditional knockout (cKO) squamous cell carcinoma's (SCCs) and human FAT1 KO SCC cell line) compared to FAT1 mutation status in human Lung SCC (TCGA database) (For the analysis only high impact mutations in >20% of variant allele frequency were considered, referred to as “FAT1 mutated”. The term “FAT1 WT” refers wild-type FAT1. Mean, the lowest and the highest values, Wilcox Rank Sum test). FIG. 2B, Box Plot showing the distribution of the common mRNA signature (mouse skin and lung Fat1cKO SCCs and human FAT1 KO SCC cell line; also referred to as “FAT1 deletion”) compared to FAT1 Copy Number Variation (CNV) status in human Lung SCC (TCGA database) (Mean±SD, Wilcox Rank Sum test).

FIG. 3 Yes-associated protein 1 (Yap1) and sex determining region Y-box 2 (Sox2) regulate mesenchymal and epithelial states downstream of Fat1 deletion. FIG. 3A, Transcription factor (TF) motifs enriched in the assay for transposase-accessible chromatin (ATAC)-sequencing peaks up-regulated between the Epcam+ Fat1cKO and Epcam+ Fat1 WT and between Epcam− Fat1cKO and Epcam− Fat1 wild-type (WT) skin squamous cell carcinoma (SCC) tumor cells (TCs) as determined by Homer. FIG. 3B, Table showing frequency of secondary tumors observed upon subcutaneous transplantation of limiting dilution of yellow fluorescent protein (YFP)+ Epcam− Fat1 KO, YFP+ Epcam− Fat1/Sox2 KO and YFP+ Epcam− Fat1/Yap1/Taz KO skin SCC TCs and the estimation of the tumor propagating cell (TPC) frequency using Extreme Limiting Dilution analysis (ELDA) (Chi-square test). FIG. 3C, Graph showing the number of lung metastasis arising from the injection of 1000 YFP+ Epcam− Fat1 KO, YFP+ Epcam− Fat1/Sox2 KO and YFP+ Epcam− Fat1/Yap1/Taz KO skin SCC TCs (Mean±standard error of the mean (SEM), two-tailed T-test). FIG. 3D, Bar chart showing the number of cells in spheroids formed by FAT1 KO, FAT1/YAP1/TAZ KO and FAT1/SOX2 KO human SCC cells 7 days after plating 4000c in ultra-low attachment plate (Mean±SEM, two-tailed T-test). FIG. 3E, Distribution of YFP+ Epcam− TC subpopulations based on CD106Ncam1, CD61/Itgb3 and CD51/Itgav expression in Fat1cKO mouse skin SCC-derived cell lines after subcutaneous transplantation (n=5). TN: triple negative (CD106−/CD51−/CD61−); TP: triple positive (CD106+/CD51+/CD61+) FIG. 3F, Distribution of YFP+ Epcam− TC subpopulations based on CD106Ncam1, CD61/Itgb3 and CD51/Itgav expression in Fat1/Sox2 KO mouse skin SCC cell lines after subcutaneous transplantation (n=6). TN: triple negative (CD106−/CD51−/CD61−); TP: triple positive (CD106+/CD51+/CD61+) FIG. 3G, Distribution of YFP+ Epcam− TC subpopulations based on CD106Ncam1, CD61/Itgb3 and CD51/Itgav expression in Fat1/Yap1/Taz1 KO mouse skin SCC cell lines after subcutaneous transplantation (n=5). TN: triple negative (CD106−/CD51−/CD61−); TP: triple positive (CD106+/CD51+/CD61+) FIG. 3H, Venn diagram of the genes upregulated in Epcam− Fat1cKO skin SCC TCs upon Sox2 deletion and naturally upregulated genes in hybrid EMT triple negative (TN) (CD106−/CD51−/CD61−) TCs as compared to late EMT triple positive (TP) CD106+/CD51+/CD61+) tumor cells (early hybrid EMT signature) and in TP as comparted to Epcam+ TCs (late EMT signature) (Two-sided hypergeometric test). FIG. 3I, Venn diagram of the genes upregulated in Epcam− Fat1cKO skin SCC TCs upon Yap1/Taz deletion and naturally upregulated genes in hybrid EMT triple negative (TN) TCs as compared to late EMT Triple Positive (TP) TCs (early hybrid EMT signature) and in TP as comparted to Epcam+ TCs (late EMT signature) (Two-sided hypergeometric test). FIG. 3J, mRNA expression of epithelial genes and genes associated with polarity controlled by Sox2 defined by RNA-seq in Epcam− Fat1cKO and Fat1/Sox2 KO skin SCC cells (Mean+SEM). FIG. 3K, mRNA expression of Yap1/Taz target genes and other mesenchymal genes regulated by Yap1/Taz defined by RNA-seq in Epcam− Fat1cKO and Fat1/Yap1/Taz KO skin SCC cells (mean+SEM) (n=2).

FIG. 4. Phosphoproteomic analysis identifies the signalling cascades downstream of FAT1 deletion. FIG. 4A, Volcano plot showing the Fold Change (in log₂ on the X-axis) of each phosphopeptide between FAT1 WT and FAT1 KO sample and statistical significance (−Log p value on the Y-axis) (t-test, fals discovery rate (FDR)=0.05 and S₀=1). FIG. 4B, Table showing the phosphorylation sites (“Ph-site”) of the different kinases significantly upregulated in FAT1 WT as compared to FAT1 KO. FC: fold-change. FIG. 4C, Western Blot showing the expression levels of phosphorylated MEK1/2 (antibody recognizes phosphorylation of MEK 1/2 on Ser218, SER222, Ser226) and total MEK in FAT1 WT and FAT1 KO cells. FIG. 4D, Western Blot showing the expression levels of phosphorylated EGFR (antibody recognizes phosphorylation of EGFR on Y1197) and total EGFR in FAT1 WT and FAT1 KO cells. FIG. 4E, Table showing the enrichment of YES1 Y194 phosphosite in FAT1 KO vs WT. FIG. 4F, Table showing the phosphosites (“Ph-site”) predicted to be phosphorylated by CAMK2 enriched in FAT1 KO cells. FC: fold-change. FIG. 4G, Western Blot showing the expression levels of phosphorylated CAMK2 (antibody recognizes phosphorylation of CAMK2alpha on Thr286, and CAMK2beta and gamma on Thr287) and total CAMK2 proteins in FAT1 WT and FAT1 KO cells. FIG. 4H, Western Blot showing the expression levels of phosphorylated SRC (antibody recognizes phosphorylation of YES/SRC on Tyr416), total SRC and YES proteins in FAT1 WT and FAT1 KO cells. FIG. 4I, Western Blot showing the expression levels of phosphorylated SRC, total SRC and YES proteins in FAT1 KO cells treated with dimethyl sulfoxide or with CAMK2 inhibitor (KN93). FIG. 4J, Histograms showing the expression of CD44 by fluorescence-activated cell sorting (FACS) in FAT1 WT and FAT1 KO TCs. FIG. 4K, Histograms showing the expression of CD44 by FACS in FAT1 KO TCs treated with DMSO or with 10 uM CAMK2 inhibitor (KN93) (n=16). FIG. 4L, Bar chart showing the proportion of FAT1 KO TCs expressing high levels of CD44 treated with DMSO or with CAMK2 inhibitor (Mean+SEM, two-tailed T-test). FIG. 4M, Western Blot showing the expression levels of phosphorylated SRC, total SRC and YES proteins in FAT1 KO and FAT1/CD44 KO TCs. FIG. 4N, Bar chart showing the quantification by FACS of the number of cells in FAT1 WT and FAT1/CD44 KO spheroids (Mean+SEM, two-tailed T-test). FIG. 4O, Western blot showing the expression levels of H3K27me3 mark and H3 proteins in FAT1 WT and FAT1 KO cells. FIG. 4P, Western Blot showing the expression levels of H3K27me3 mark and H3 proteins in FAT1 KO TCs treated with DMSO or with CAMK2 inhibitor (KN93). FIG. 4Q, Western Blot showing the expression levels of H3K27me3 mark and H3 proteins in FAT1 WT cells treated with DMSO or with EZH2 inhibitor (GSK343). FIG. 4R, Dot plot showing SOX2 mRNA expression by RT-qPCR in FAT1 WT cells treated during 7 days with DMSO or with EZH2-inhibitor (GSK343) (Mean±SEM, two-tailed T-test). FC: fold-change. FIG. 4S, Western Blot showing the expression level of SOX2 protein in FAT1 WT cells treated during 7 days with DMSO or with EZH2-inhibitor (GSK343). FIG. 4T, ChIP-qPCR showing different levels of H3K27me3 mark deposition in the genomic region surrounding SOX2 TSS in FAT1 WT and KO cells. The data represent the ratio of relative enrichment of the indicated genomic regions in Fat1 WT vs KO cells. Ctrl1 and 2 are negative control regions for EZH2/H3K27me3. (One sample T-test, Mean±SEM. Control 1 n=3, p=0.61; Control 2 n=3, p=0.63; −0.5 n=3, p=0.04; 0.1 n=3, p=0.008; 0.5 n=3, p=0.006; 0.7 n=3, p=0.02). FIG. 4U, Representative dose-response curve showing the effect of increasing doses of EGFR-inhibitor Afatinib on FAT1 WT and FAT1 KO cell viability at 48 h analysed by FACS (normalized to the average of 5 wells treated with DMSO). Non-linear regression log(inhibitor) with least squares fit method (n=3, Mean±SEM). FIG. 4V, Representative dose-response curve showing the effect of increasing doses of SRC-inhibitor Dasatinib on FAT1 WT and FAT1 KO cell viability at 48 hours analysed by FACS (normalized to the average of 4 wells treated with DMSO). Non-linear regression log(inhibitor) with least squares fit method (n=3, Mean±SEM). FIG. 4W, Table showing the summary (n=3) of pIC50 and SEM for different drugs for FAT1 WT and FAT1 KO cells (two-tailed T-test).

FIG. 5 Phosphoproteomic analysis reveal signalling cascades downstream of FAT1 loss of function. FIG. 5A, Table showing kinases significantly more phosphorylated in FAT1 KO cells as compared to FAT1 WT. FIG. 5B, Bar chart showing the kinases that are predicted to phosphorylate phosphosites significantly enriched in FAT1 KO TCs. FIG. 5C, Table showing kinases significantly more phosphorylated in FAT1 WT cells as compared to FAT1 KO. FIG. 5D, Bar chart showing the kinases that are predicted to phosphorylate phosphosites significantly enriched in FAT1 WT TCs.

FIG. 6 Increase in Yap1 and Sox2 signalling downstream of FAT1 LOF is independent of the stiffness of the substrate. FIG. 6A, Quantification of YAP1 expression based on fluorescence intensity in FAT1 WT and FAT1 KO cells upon different stiffness conditions. FIG. 6B, Quantification of YAP1 nuclear/cytoplasmic ratio based on fluorescence intensity in FAT1 WT and FAT1 KO cells upon different stiffness conditions. FIG. 6C, Schematic showing the signaling pathways that are activated in FAT1 WT and FAT1 KO cells and predict a differential impact on the response to therapy.

FIG. 7 Table summarizing the samples of Patient Derived Xenografts (PDX) and the detailed information on the mutations: codon, amino acid (AA) change, the exon harbouring the mutation, the allelic frequency, the type of mutations and the bioinformatic prediction of the impact of the mutation on the function of the protein by 3 different bioinformatic algorithms (SIFT, Memo and PolyPhen)

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The inventors have realised that loss of FAT1 expression or function promotes the acquisition of a hybrid epithelial to mesenchymal transition (EMT) state presenting increased tumor stemness and metastasis. Furthermore, the inventors found, by a comprehensive molecular characterization including transcriptomic, epigenomic and proteomic characterization of Fat1 mutants, that the hybrid EMT signature is mediated by the activation of yes-associated protein 1 (YAP1) and sex determining region Y-box2 (Sox2), and that several signaling cascades involving epidermal growth factor receptor (EGFR), mitogen-activated protein kinase (MEK), Ca²⁺/calmodulin-dependent protein kinase (CAMK) and SRC, are responsible for said activation. Furthermore, the inventors found that FAT1 can be used as a clinical marker for determining the sensitivity of subjects to antineoplastic agents which modulate one or more signaling cascades which are impacted by reduced or abolished FAT1 expression or function, such as an EGFR inhibitor, a MEK inhibitor, a CAMK inhibitor, or a SRC kinase inhibitor. Loss of FAT1 expression or function may be caused by, for example, a change in the FAT1 sequence (e.g. FAT1 truncation), a change in the FAT1 structure, a change in FAT1 transport, FAT1 misfolding, a decrease in the FAT1 mRNA stability or translation, a mutation in the promoter or enhancer of FAT1 causing a decreased FAT1 expression, compared to wild-type FAT1.

An aspect thus provides a method for determining sensitivity (which may also be denoted as responsiveness or susceptibility) or resistance (which may also be denoted as unresponsiveness or insusceptibility) to treatment with an antineoplastic agent in a subject diagnosed with a neoplastic disease, the method comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject;
   wherein said antineoplastic agent is selected from the group consisting of an EGFR inhibitor, a MEK inhibitor, a CAMK inhibitor, and a SRC kinase inhibitor. In other words, this aspect employs FAT1 status as a marker for determining sensitivity or resistance to treatment with certain antineoplastic agents in a subject diagnosed with a neoplastic disease.

In certain embodiments, reduced or abolished FAT1 expression or function (e.g. loss of FAT1 expression or function), may be employed as a marker for the subject's sensitivity to treatment with certain antineoplastic agent(s).

In certain embodiments, normal FAT1 expression or function may be employed as a marker for the subject's sensitivity to treatment with certain other antineoplastic agent(s). The terms “marker” and “biomarker” are widespread in the art and commonly broadly denote a biological component or a biological molecule, more particularly an endogenous biological component or molecule, or a detectable portion thereof, whose qualitative and/or quantitative evaluation in a tested subject, such as by means of evaluating a biological sample from the subject, is predictive or informative with respect to one or more aspects of the tested subjects' phenotype and/or genotype. The phrases “determining sensitivity” and “predicting sensitivity” may be used interchangeably herein.

The terms “predicting”, “prediction” or “predictive” as used herein refers to an advance declaration, indication or foretelling of a response or reaction to a therapy in a subject, preferably wherein said subject has not (yet) been treated with the therapy. For example, a prediction of sensitivity (or responsiveness or susceptibility) to treatment with an antineoplastic agent in a subject may indicate that the subject will respond or react to the treatment, for example within a certain time period, e.g., so that the subject will have a clinical benefit (e.g., will display reduced tumor load) from the treatment. A prediction of insensitivity (or unresponsiveness or insusceptibility or resistance) to treatment with an antineoplastic agent in a subject may indicate that the subject will minimally or not respond or react to the treatment, for example within a certain time period, e.g., so that the subject will have no clinical benefit (e.g., will not display a therapeutically meaningful reduction in tumor load) from the treatment.

The terms “sensitivity”, “responsiveness” or “susceptibility” may be used interchangeably herein and refer to the quality that predisposes a subject having a neoplastic disease to be sensitive or reactive to treatment with an antineoplastic agent. A subject is “sensitive”, “responsive” or “susceptible” (which terms may be used interchangeably) to treatment with an antineoplastic agent if the subject will have a clinical benefit from the treatment. A neoplastic tissue, such as a tumor, is “sensitive”, “responsive”, or “susceptible” to treatment with an antineoplastic agent if the proliferation rate of the neoplastic tissue is inhibited as a result of contact with a therapeutically effective amount of the antineoplastic agent, compared to the proliferation rate of the neoplastic tissue in the absence of contact with the antineoplastic agent.

The terms “insensitivity”, “unresponsiveness”, “insusceptibility” or “resistance” may be used interchangeably herein and refer to the quality that predisposes a subject having a neoplastic disease to a minimal (e.g. clinically insignificant) or no response to treatment with an antineoplastic agent. A subject is “insensitive”, “unresponsive”, “unsusceptible” or “resistant” (which terms may be used interchangeably) to treatment with an antineoplastic agent if the subject will have no clinical benefit from the treatment. A neoplastic tissue, including a tumor, is “insensitive”, “unresponsive”, “unsusceptible” or “resistant” to treatment with an antineoplastic agent if the proliferation rate of the neoplastic tissue is not inhibited, or inhibited to a very low (e.g. therapeutically insignificant) degree, as a result of contact with a therapeutically effective amount of the antineoplastic agent, compared to the proliferation rate of the neoplastic tissue in the absence of contact with the antineoplastic agent.

The methods as disclosed herein may allow making a prediction that a subject having a neoplastic disease will be sensitive to treatment with an antineoplastic agent or will be resistant to treatment with an antineoplastic agent. This may in certain embodiments include predicting that a subject having a neoplastic disease will have a comparatively low probability (e.g., less than 50%, less than 40%, less than 30%, less than 20% or less than 10%) of being sensitive to treatment with an antineoplastic agent; or that a subject having a neoplastic disease will have a comparatively high probability (e.g., at least 50%, at least 60%, at least 70%, at least 80% or at least 90%) of being sensitive to treatment with an antineoplastic agent.

The present methods evaluating FAT1 status to provide information as to the subject's sensitivity or resistance to a given antineoplastic treatment are generally performed in vitro, on a sample (such as a tissue biopsy or liquid biopsy) obtained from a subject. The term “in vitro” generally denotes outside, or external to, animal or human body. The term “ex vivo” typically refers to tissues or cells removed from an animal or human body and maintained or propagated outside the body, e.g., in a culture vessel.

The term “in vitro” as used herein should be understood to include “ex vivo”. The term “in vivo” generally denotes inside, on, or internal to, animal or human body. FAT1 is member of the cadherin superfamily, which is a group of integral membrane proteins characterized by the presence of cadherin-type repeats. FAT1 has also been described as a tumor suppressor gene or tumor suppressor. The reference to “FAT1” denotes the FAT1 protein, polypeptide, peptide, or nucleic acid, as commonly known under said designation in the art. By means of additional guidance, FAT1 is also known as related tumor suppressor homolog, cadherin-related family member 8, cadherin family member 7, CDHF7 or CDHR8. The term “FAT1 polypeptide” as used herein is synonymous with “FAT1 protein”. Sometimes, adjectives such as “unmodified”, “unchanged”, “original”, “starting”, “non-mutated”, may be used in conjunction with the term “FAT1” to emphasise the distinction between FAT1 and its mutants. In certain embodiments, FAT1 may be the “wild-type” protein in its conventional meaning of the form encoded by the allele of the respective gene that is most commonly observed in a population. In certain embodiments, FAT1 may be the “wild-type” protein in its phenotype-oriented meaning of any form that is not causative of or associated with an altered phenotype such as a disease. By means of an illustration and without limitation, a wild-type FAT1 allele will produce a functional, full-length FAT1 protein, whereas a mutant FAT1 allele may produce no FAT1 mRNA or FAT1 protein, or may produce FAT1 mRNA encoding a truncated FAT1 protein, which may optionally contain non-FAT1 sequences resulting from a reading frame shift (such mutant FAT1 proteins may be non-functional and may often be directed towards fast degradation), or may produce a FAT1 protein containing a structurally comparatively minor mutation (e.g., a single amino acid substitution) which nevertheless substantially reduces or entirely abolishes the normal function of the FAT1 protein in the cell.

The term “nucleic acid” s used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Preferably, ribonucleic acids or RNA may be composed primarily of ribonucleoside units, for example, ≥80%, ≥85%, ≥90%, ≥95%, ≥96%, ≥97%, ≥98%, ≥99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H. Preferably, deoxyribonucleic acids or DNA may be composed primarily of deoxyribonucleoside units, for example, ≥80%, ≥85%, ≥90%, ≥95%, ≥96%, ≥97%, ≥98%, ≥99% or even 100% (by number) of nucleoside units constituting the nucleic acid molecule may be deoxyribonucleoside units. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Also included are amino acid analogues, in which one or more individual atoms have been replaced either with a different atom, an isotope of the same atom, or with a different functional group. The reference herein to any nucleic acid, protein, polypeptide or peptide may also encompass variants thereof. The term “variant” of a nucleic acid, protein, polypeptide or peptide refers to nucleic acids, proteins, polypeptides or peptides the sequence (i.e., nucleotide sequence or amino acid sequence, respectively) of which is substantially identical (i.e., largely but not wholly identical) to the sequence of said recited nucleic acid, protein or polypeptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited nucleic acid, protein, polypeptide or peptide when the whole sequence of the recited nucleic acid, protein, polypeptide or peptide is queried in the sequence alignment (i.e., overall sequence identity). Also included among fragments and variants of a nucleic acid, protein, polypeptide or peptide are fusion products of said nucleic acid, protein, polypeptide or peptide with another, usually unrelated, nucleic acid, protein, polypeptide or peptide. Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3). A variant of a nucleic acid, protein, polypeptide or peptide may be a homologue (e.g., orthologue or paralogue) of said nucleic acid, protein, polypeptide or peptide. As used herein, the term “homology” generally denotes structural similarity between two macromolecules, particularly between two nucleic acids, proteins or polypeptides, from same or different taxons, wherein said similarity is due to shared ancestry. The reference herein to any peptide, polypeptide, protein, or nucleic acid also encompasses fragments thereof. Hence, the reference herein to measuring (or measuring the quantity of) any one peptide, polypeptide, protein, or nucleic acid may encompass measuring the peptide, polypeptide, protein, or nucleic acid, such as, e.g., measuring any mature and/or processed soluble/secreted form(s) thereof (e.g., plasma circulating form(s)) and/or measuring one or more fragments thereof. For example, any peptide, polypeptide, protein, or nucleic acid, and/or one or more fragments thereof may be measured collectively, such that the measured quantity corresponds to the sum amounts of the collectively measured species. In another example, any peptide, polypeptide, protein, or nucleic acid, and/or one or more fragments thereof may be measured each individually.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of 5 consecutive nucleotides, or ≥10 consecutive nucleotides, or ≥20 consecutive nucleotides, or ≥30 consecutive nucleotides, e.g., ≥40 consecutive nucleotides, such as for example 50 consecutive nucleotides, e.g., ≥60, ≥70, ≥80, ≥90, ≥100, ≥200, ≥300, ≥400, ≥500, ≥1000, ≥2000, ≥3000, ≥4000, ≥5000, ≥6000, ≥7000, ≥8000, ≥9000, ≥10000, ≥11000, ≥12000, ≥13000 or ≥14000 consecutive nucleotides of the corresponding full-length nucleic acid.

The term encompasses fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The term “fragment” with reference to a peptide, polypeptide, or protein generally denotes a N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., consecutive amino acids, such as for example ≥50 consecutive amino acids, e.g., ≥60, ≥70, ≥80, ≥90, ≥100, ≥200, ≥300, ≥400, ≥500, ≥1000, ≥1500, ≥2000, ≥2500, ≥3000, ≥3500, ≥4000 or ≥4500 consecutive amino acids of the corresponding full-length peptide, polypeptide, or protein.

The reference to any peptide, polypeptide, protein, or nucleic acid, corresponds to the peptide, polypeptide, protein, nucleic acid, commonly known under the respective designations in the art. The terms encompass such peptides, polypeptides, proteins, or nucleic acids of any organism where found, and particularly of animals, preferably warm-blooded animals, more preferably vertebrates, yet more preferably mammals, including humans and non-human mammals, still more preferably of humans. The terms particularly encompass such peptides, polypeptides, proteins, or nucleic acids with a native sequence, i.e., ones of which the primary sequence is the same as that of the peptides, polypeptides, proteins, or nucleic acids found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or post-translational modifications. Any such variants or isoforms of peptides, polypeptides, proteins, or nucleic acids are intended herein. Accordingly, all sequences of peptides, polypeptides, proteins, or nucleic acids found in or derived from nature are considered “native”. The terms encompass the peptides, polypeptides, proteins, or nucleic acids when forming a part of a living organism, organ, tissue or cell, when forming a part of a biological sample, as well as when at least partly isolated from such sources.

In certain embodiments, peptides, polypeptides, proteins, or nucleic acids may be human, i.e., their primary sequence may be the same as a corresponding primary sequence of or present in a naturally occurring human peptides, polypeptides, proteins, or nucleic acids. Hence, the qualifier “human” in this connection relates to the primary sequence of the respective peptides, polypeptides, proteins, or nucleic acids, rather than to its origin or source. For example, such peptides, polypeptides, proteins, or nucleic acids may be present in or isolated from samples of human subjects or may be obtained by other means (e.g., by recombinant expression, cell-free transcription or translation, or non-biological nucleic acid or peptide synthesis).

In particular embodiments, FAT1 is human FAT1. Exemplary human FAT1 protein sequence may be as annotated under U.S. government's National Center for Biotechnology Information (NCBI) Genbank (http://www.ncbi.nlm.nih.gov/) accession number NP_005236.2, or Swissprot/Uniprot (http://www.uniprot.org/) accession number Q14517.2. The sequence annotated under NCBI Genbank accession number NP_005236.2 is reproduced below:

>NP_005236.2 protocadherin Fat 1 precursor
[Homo sapiens]
(SEQ ID NO: 1)
MGRHLALLLLLLLLFQHFGDSDGSQRLEQTPLQFTHLEYNVTVQENSAAKTYVGHPVKMGVYI
THPAWEVRYKIVSGDSENLFKAEEYILGDFCFLRIRTKGGNTAILNREVKDHYTLIVKALEKN
TNVEARTKVRVQVLDTNDLRPLFSPTSYSVSLPENTAIRTSIARVSATDADIGTNGEFYYSFK
DRTDMFAIHPTSGVIVLTGRLDYLETKLYEMEILAADRGMKLYGSSGISSMAKLTVHIEQANE
CAPVITAVTLSPSELDRDPAYAIVTVDDCDQGANGDIASLSIVAGDLLQQFRTVRSFPGSKEY
KVKAIGGIDWDSHPFGYNLTLQAKDKGTPPQFSSVKVIHVTSPQFKAGPVKFEKDVYRAEISE
FAPPNTPVVMVKAIPAYSHLRYVFKSTPGKAKFSLNYNTGLISILEPVKRQQAAHFELEVTTS
DRKASTKVLVKVLGANSNPPEFTQTAYKAAFDENVPIGTTVMSLSAVDPDEGENGYVTYSIAN
LNHVPFAIDHFTGAVSTSENLDYELMPRVYTLRIRASDWGLPYRREVEVLATITLNNLNDNTP
LFEKINCEGTIPRDLGVGEQITTVSAIDADELQLVQYQIEAGNELDFFSLNPNSGVLSLKRSL
MDGLGAKVSFHSLRITATDGENFATPLYINITVAASHKLVNLQCEETGVAKMLAEKLLQANKL
HNQGEVEDIFFDSHSVNAHIPQFRSTLPTGIQVKENQPVGSSVIFMNSTDLDTGFNGKLVYAV
SGGNEDSCFMIDMETGMLKILSPLDRETTDKYTLNITVYDLGIPQKAAWRLLHVVVVDANDNP
PEFLQESYFVEVSEDKEVHSEIIQVEATDKDLGPNGHVTYSIVTDTDTFSIDSVTGVVNIARP
LDRELQHEHSLKIEARDQAREEPQLFSTVVVKVSLEDVNDNPPTFIPPNYRVKVREDLPEGTV
IMWLEAHDPDLGQSGQVRYSLLDHGEGNFDVDKLSGAVRIVQQLDFEKKQVYNLTVRAKDKGK
PVSLSSTCYVEVEVVDVNENLHPPVFSSFVEKGTVKEDAPVGSLVMTVSAHDEDARRDGEIRY
SIRDGSGVGVFKIGEETGVIETSDRLDRESTSHYWLTVFATDQGVVPLSSFIEIYIEVEDVND
NAPQTSEPVYYPEIMENSPKDVSVVQIEAFDPDSSSNDKLMYKITSGNPQGFFSIHPKTGLIT
TTSRKLDREQQDEHILEVTVTDNGSPPKSTIARVIVKILDENDNKPQFLQKFYKIRLPEREKP
DRERNARREPLYHVIATDKDEGPNAEISYSIEDGNEHGKFFIEPKTGVVSSKRFSAAGEYDIL
SIKAVDNGRPQKSSTTRLHIEWISKPKPSLEPISFEESFFTFTVMESDPVAHMIGVISVEPPG
IPLWFDITGGNYDSHFDVDKGTGTIIVAKPLDAEQKSNYNLTVEATDGTTTILTQVFIKVIDT
NDHRPQFSTSKYEVVIPEDTAPETEILQISAVDQDEKNKLIYTLQSSRDPLSLKKFRLDPATG
SLYTSEKLDHEAVHQHTLTVMVRDQDVPVKRNFARIVVNVSDTNDHAPWFTASSYKGRVYESA
AVGSVVLQVTALDKDKGKNAEVLYSIESGNIGNSFMIDPVLGSIKTAKELDRSNQAEYDLMVK
ATDKGSPPMSEITSVRIFVTIADNASPKFTSKEYSVELSETVSIGSFVGMVTAHSQSSVVYEI
KDGNTGDAFDINPHSGTIITQKALDFETLPIYTLIIQGTNMAGLSTNTTVLVHLQDENDNAPV
FMQAEYTGLISESASINSVVLTDRNVPLVIRAADADKDSNALLVYHIVEPSVHTYFAIDSSTG
AIHTVLSLDYEETSIFHFTVQVHDMGTPRLFAEYAANVTVHVIDINDCPPVFAKPLYEASLLL
PTYKGVKVITVNATDADSSAFSQLIYSITEGNIGEKFSMDYKTGALTVQNTTQLRSRYELTVR
ASDGRFAGLTSVKINVKESKESHLKFTQDVYSAVVKENSTEAETLAVITAIGNPINEPLFYHI
LNPDRRFKISRTSGVLSTTGTPFDREQQEAFDVVVEVTEEHKPSAVAHVVVKVIVEDQNDNAP
VFVNLPYYAVVKVDTEVGHVIRYVTAVDRDSGRNGEVHYYLKEHHEHFQIGPLGEISLKKQFE
LDTLNKEYLVTVVAKDGGNPAFSAEVIVPITVMNKAMPVFEKPFYSAEIAESIQVHSPVVHVQ
ANSPEGLKVFYSITDGDPFSQFTINFNTGVINVIAPLDFEAHPAYKLSIRATDSLTGAHAEVF
VDIIVDDINDNPPVFAQQSYAVTLSEASVIGTSVVQVRATDSDSEPNRGISYQMFGNHSKSHD
HFHVDSSTGLISLLRTLDYEQSRQHTIFVRAVDGGMPTLSSDVIVTVDVTDLNDNPPLFEQQI
YEARISEHAPHGHFVTCVKAYDADSSDIDKLQYSILSGNDHKHFVIDSATGIITLSNLHRHAL
KPFYSLNLSVSDGVFRSSTQVHVTVIGGNLHSPAFLQNEYEVELAENAPLHTLVMEVKTTDGD
SGIYGHVTYHIVNDFAKDRFYINERGQIFTLEKLDRETPAEKVISVRLMAKDAGGKVAFCTVN
VILTDDNDNAPQFRATKYEVNIGSSAAKGTSVVKVLASDADEGSNADITYAIEADSESVKENL
EINKLSGVITTKESLIGLENEFFTFFVRAVDNGSPSKESVVLVYVKILPPEMQLPKFSEPFYT
FTVSEDVPIGTEIDLIRAEHSGTVLYSLVKGNTPESNRDESFVIDRQSGRLKLEKSLDHETTK
WYQFSILARCTQDDHEMVASVDVSIQVKDANDNSPVFESSPYEAFIVENLPGGSRVIQIRASD
ADSGTNGQVMYSLDQSQSVEVIESFAINMETGWITTLKELDHEKRDNYQIKVVASDHGEKIQL
SSTAIVDVTVTDVNDSPPRFTAEIYKGTVSEDDPQGGVIAILSTTDADSEEINRQVTYFITGG
DPLGQFAVETIQNEWKVYVKKPLDREKRDNYLLTITATDGTFSSKAIVEVKVLDANDNSPVCE
KTLYSDTIPEDVLPGKLIMQISATDADIRSNAEITYTLLGSGAEKFKLNPDTGELKTSTPLDR
EEQAVYHLLVRATDGGGRFCQASIVLTLEDVNDNAPEFSADPYAITVFENTEPGTLLTRVQAT
DADAGLNRKILYSLIDSADGQFSINELSGIIQLEKPLDRELQAVYTLSLKAVDQGLPRRLTAT
GTVIVSVLDINDNPPVFEYREYGATVSEDILVGTEVLQVYAASRDIEANAEITYSIISGNEHG
KFSIDSKTGAVFIIENLDYESSHEYYLTVEATDGGTPSLSDVATVNVNVTDINDNTPVFSQDT
YTTVISEDAVLEQSVITVMADDADGPSNSHIHYSIIDGNQGSSFTIDPVRGEVKVTKLLDRET
ISGYTLTVQASDNGSPPRVNTTTVNIDVSDVNDNAPVFSRGNYSVIIQENKPVGFSVLQLVVT
DEDSSHNGPPFFFTIVTGNDEKAFEVNPQGVLLTSSAIKRKEKDHYLLQVKVADNGKPQLSSL
TYIDIRVIEESIYPPAILPLEIFITSSGEEYSGGVIGKIHATDQDVYDTLTYSLDPQMDNLFS
VSSTGGKLIAHKKLDIGQYLLNVSVTDGKFTTVADITVHIRQVTQEMLNHTIAIRFANLTPEE
FVGDYWRNFQRALRNILGVRRNDIQIVSLQSSEPHPHLDVLLFVEKPGSAQISTKQLLHKINS
SVTDIEEIIGVRILNVFQKLCAGLDCPWKFCDEKVSVDESVMSTHSTARLSFVTPRHHRAAVC
LCKEGRCPPVHHGCEDDPCPEGSECVSDPWEEKHTCVCPSGRFGQCPGSSSMTLTGNSYVKYR
LTENENKLEMKLTMRLRTYSTHAVVMYARGTDYSILEIHHGRLQYKFDCGSGPGIVSVQSIQV
NDGQWHAVALEVNGNYARLVLDQVHTASGTAPGTLKTLNLDNYVFFGGHIRQQGTRHGRSPQV
GNGFRGCMDSIYLNGQELPLNSKPRSYAHIEESVDVSPGCFLTATEDCASNPCQNGGVCNPSP
AGGYYCKCSALYIGTHCEISVNPCSSKPCLYGGTCVVDNGGFVCQCRGLYTGQRCQLSPYCKD
EPCKNGGTCFDSLDGAVCQCDSGFRGERCQSDIDECSGNPCLHGALCENTHGSYHCNCSHEYR
GRHCEDAAPNQYVSTPWNIGLAEGIGIVVFVAGIFLLVVVFVLCRKMISRKKKHQAEPKDKHL
GPATAFLQRPYFDSKLNKNIYSDIPPQVPVRPISYTPSIPSDSRNNLDRNSFEGSAIPEHPEF
STFNPESVHGHRKAVAVCSVAPNLPPPPPSNSPSDSDSIQKPSWDFDYDTKVVDLDPCLSKKP
LEEKPSQPYSARESLSEVQSLSSFQSESCDDNGYHWDTSDWMPSVPLPDIQEFPNYEVIDEQT
PLYSADPNAIDTDYYPGGYDIESDFPPPPEDFPAADELPPLPPEFSNQFESIHPPRDMPAAGS
LGSSSRNRQRFNLNQYLPNFYPLDMSEPQTKGTGENSTCREPHAPYPPGYQRHFEAPAVESMP
MSVYASTASCSDVSACCEVESEVMMSDYESGDDGHFEEVTIPPLDSQQHTEV.

A skilled person can appreciate that any sequences represented in sequence databases or in the present specification may be of precursors of the respective peptides, polypeptides, proteins or nucleic acids and may include parts which are processed away from mature molecules.

The mature form of human FAT1 protein (not comprising the signal peptide) may thus in a certain embodiment comprise an amino sequence as shown in SEQ ID NO: 1, from which the first 21 consecutive amino acids (i.e., the signal peptide MGRHLALLLLLLLLFQHFGDS (SEQ ID NO: 2)) are removed or excised.

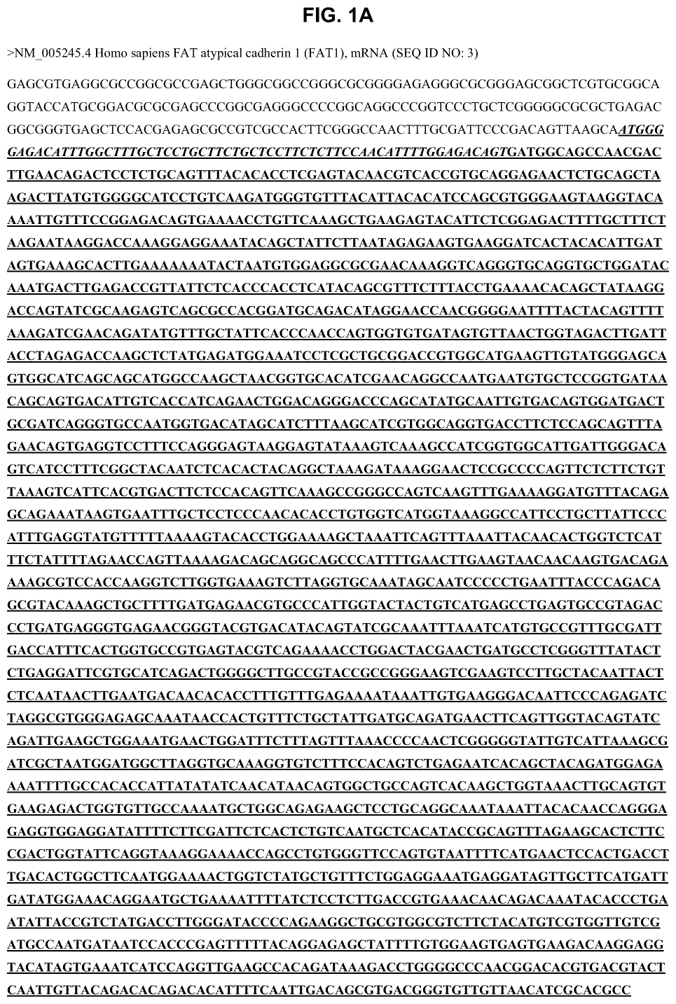

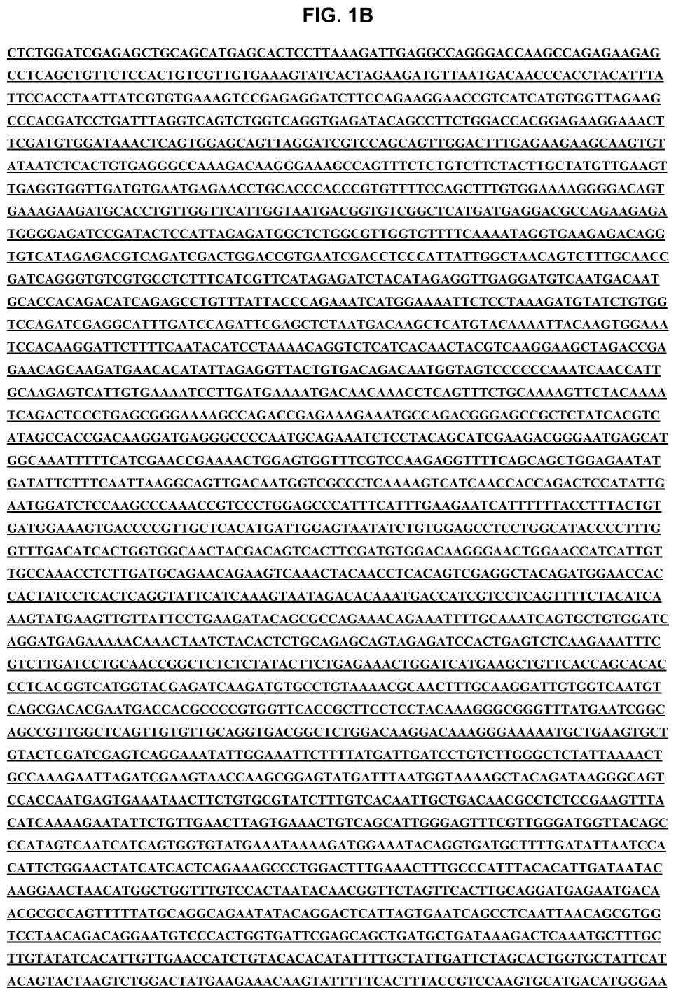

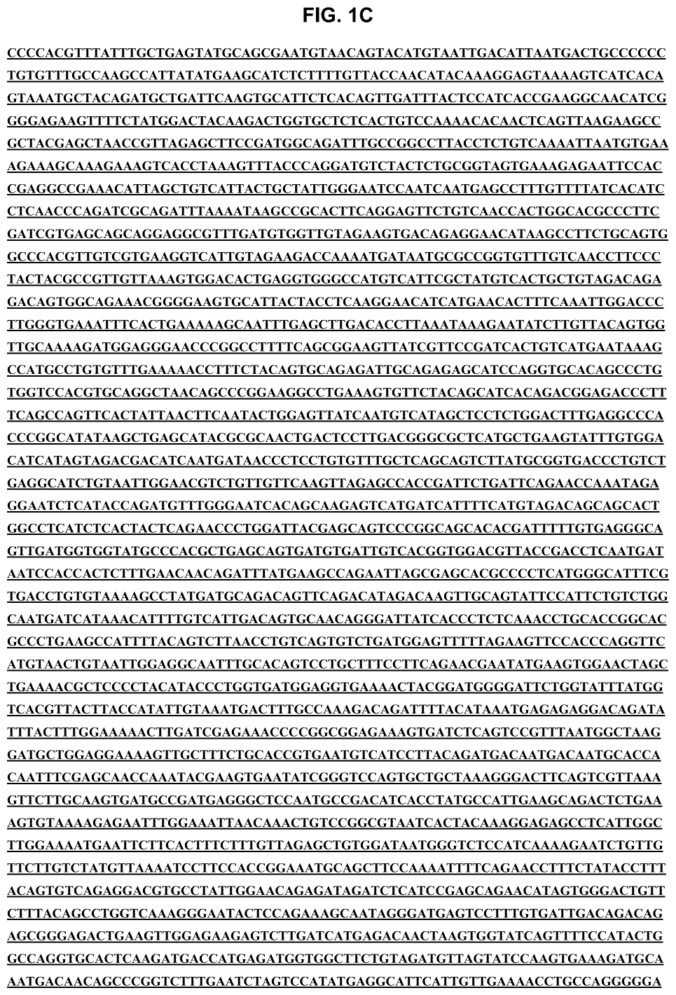

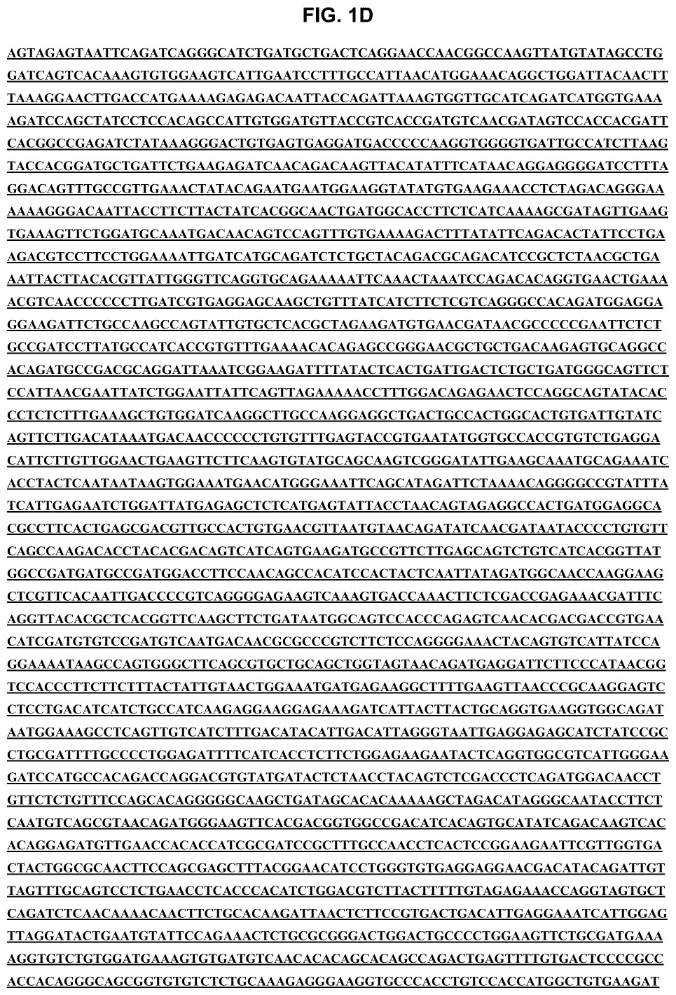

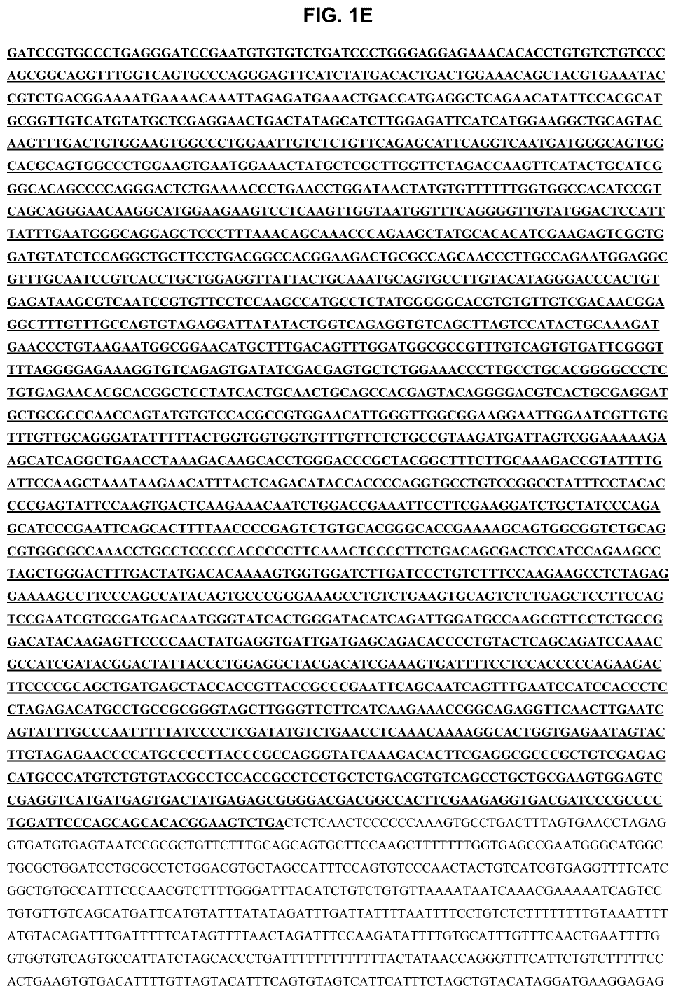

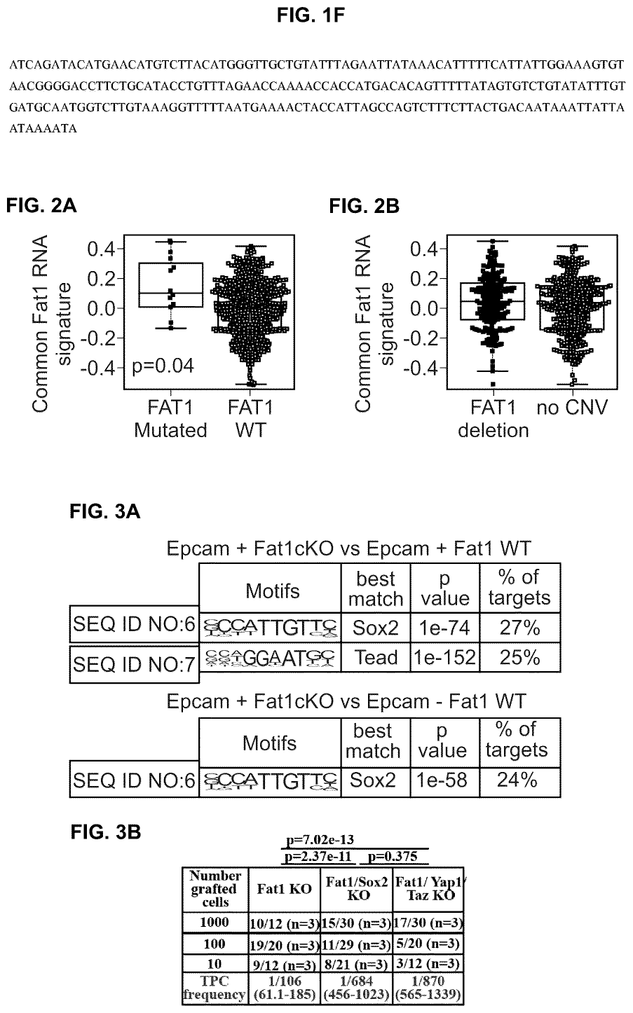

Exemplary human FAT1 mRNA (cDNA) sequence may be as annotated under NCBI Genbank accession number NM 005245.4. The NM 005245.4 nucleic acid sequence is reproduced in FIG. 1.

As described above, the present invention concerns assessment of the sensitivity or resistance to certain antineoplastic agents based on the analysis of FAT1 status, such as based on genetic, epigenetic, expression or functional analysis informative of FAT1 status. A genetic alteration may produce an alteration in the FAT1 protein's expression or function through, for example, changes in the sequence, structure, stability or transport of the FAT1 protein, or any combination thereof, compared to the sequence, structure, stability or transport of a non-mutated or wild-type FAT1 protein.

More particularly, substitutions, deletions and/or insertions in the FAT1 gene (including in those parts of the FAT1 gene that encode the FAT1 protein, i.e., the coding sequence, as well as in non-coding parts of the FAT1 gene, such as inter alia any regulatory sequences, for example in the FAT1 gene promoter or any enhancers, the 5′ and 3′ untranslated regions (UTRs), or any intragenic regions (introns) separating FAT1 exons) can alter the amino acid sequence of the FAT1 protein as described elsewhere herein.

Genetic alterations that may lead to reduced or abolished expression or function of FAT1 include small-scale mutations, such as substitutions, inversions, insertions and deletions, as well as large-scale mutations, such as copy number aberrations (CNA) and chromosomal aberrations. The term “deletion” as used herein with regard to genetic alterations refers to a mutation wherein one or more nucleotides, typically consecutive nucleotides, of a nucleic acid are removed, i.e., deleted, from the nucleic acid. The term “insertion” as used herein with regard to genetic alterations refers to a mutation wherein one or more nucleotides, typically consecutive nucleotides, are added, i.e., inserted, into a nucleic acid. The term “substitution” as used herein with regard to genetic alterations refers to a mutation wherein one or more nucleotides of a nucleic acid are each independently replaced, i.e., substituted, by another nucleotide.

In certain embodiments, the mutation may be a point mutation, in which a single nucleotide base is substituted, inserted or deleted. Single nucleotide mutations are a frequent category among genetic alterations, and may have various consequences, for example leading to reading frame shift in case of single nucleotide deletions and insertions in the coding sequence, or leading to single amino acid substitutions or to a premature stop codon in case of single nucleotide substitutions in the coding sequence.

Genetic alterations as intended herein are particularly somatic alterations or mutations, i.e., an acquired alteration or mutation in DNA sequence of a subject that occurs after conception, such as which occurs in cells or a precancerous lesion or in cells of a tumor or cancer present in the subject.

Furthermore, diploid organisms, such as humans, carry two copies of each gene located on autosomes (non-sex chromosomes; note that FAT1 is located on the long arm of chromosome 4), i.e., two alleles of such genes. Diploid organisms may carry identical alleles (i.e., may be homozygous for a gene), or carry different alleles (i.e., may be heterozygous for a gene). Where a region of an autosome normally containing a given gene is deleted in a cell, such as due to a large-scale somatic chromosomal rearrangement affecting that autosome, the cell carrying only a single allele of the gene can be denoted as hemizygous for the gene. Two different alleles may be for instance a non-mutated (wild-type) and a mutated allele. An alteration or mutation in a gene may be a recessive alteration or mutation or a dominant alteration or mutation. Recessive mutations may lead to a loss of function, which may be partly or completely masked if a second allele provides a normal, non-mutated, copy of the gene. In case of recessive mutations, the subject, or certain diseased cells or tissues of the subject, can be heterozygous while still showing a (partial) mutant phenotype or the subject, or certain diseased cells or tissues of the subject, may need to be homozygous for the same or different recessive mutations in order to show the mutant phenotype. On the other hand, dominant mutations lead to a mutant phenotype in the presence of a normal copy of the gene. In addition, “dominant negative” mutations may produce a mutant form of a protein which acts antagonistically to the unmodified protein. Hence, not only do such dominant negative mutations impair the function of the mutant protein, but the mutant protein also hampers or eliminates the function of the wild-type protein, for instance by forming an inactive complex with the latter, or by still engaging with cellular partners or in cellular processes as the wild-type protein would but without inducing the normal consequences of such engagement.

In particular embodiments, the subject, or certain diseased cells or tissues of the subject, may be heterozygous or homozygous for the genetic alteration leading to a reduced or abolished expression or function of FAT1.

In particular embodiments, for the genetic alteration to lead to a reduced or abolished expression or function of FAT1 in a cell, the genetic alteration as intended herein may be a recessive mutation or a dominant mutation, such as a dominant-negative mutation.

In particular embodiments, for the genetic alteration to lead to a reduced or abolished expression or function of FAT1 in a cell the genetic alteration may occur in only one allele of the FAT1 gene in the cell while the other allele is a wild-type allele.

In particular embodiments, for the genetic alteration to lead to a reduced or abolished expression or function of FAT1 in a cell the genetic alteration (the same or different genetic alteration) may occur in both alleles of the FAT1 gene in the cell.

In particular embodiments, the methods as taught herein comprise determining in a biological sample obtained from said subject the presence or absence of a genetic or epigenetic alteration leading to reduced or abolished expression or function of FAT1 in one or both FAT1 alleles.

In particular embodiments, the methods as taught herein comprises determining in a biological sample obtained from said subject the presence or absence of one or more FAT1 loss-of-function (LOF) mutations.

The term “FAT1 loss-of-function mutation” or “FAT1 LOF mutation” as used herein refers to an alteration in the FAT1 gene which causes a statistically significant reduction (or decrease or downregulation) of the expression and/or function of the FAT1 gene product compared to the expression and/or friction of a reference. The skilled person is able to select such a reference. An example of a suitable reference may be the expression and/or function of the corresponding non-mutated (wild-type) FAT1 gene product. For example, such decrease may fall outside of error margins for the reference (as expressed, for example, by standard deviation or standard error, or by a predetermined multiple thereof, e.g., ±1×SD or ±2×SD, or ±1×SE or ±2×SE). By means of an illustration, the expression and/or function of the FAT1 gene product may be considered decreased when it is decreased such as by at least 10%, at least 20%, at least 30%, at least 40%, preferably at least 50%, at least 60%, at least 70%, more preferably at least 75%, at least 80%, at least 85%, even more preferably at least 90%, and particularly preferably at least 95%, such as 96%, 97% or 98%, or at least 99%, up to and including a 100% decrease (i.e. a complete elimination), compared to the expression and/or function of a reference.

In certain preferred embodiments, the FAT1 LOF mutation may be a point mutation, such as a point mutation resulting in a single amino acid substitution in the FAT1 protein or introducing a premature stop codon leading to a C-terminally truncated FAT1 protein, or a point mutation resulting in a reading frame shift in the FAT1 mRNA and consequently in a FAT1 protein containing a C-terminal non-FAT1 amino acid sequence often ending at an out-of-frame stop codon.

FAT1 mutations are well known in the art. Human genes and proteins are extensively annotated inter aria in the aforementioned Genbank and Uniprot databases. Known variants and mutants (including isoforms, polymorphic forms, disease-causing or associated mutants, etc.) of the human FAT1 gene or gene product are also annotated therein, for example in the NCBI Genbank variation viewer for the human FAT1 gene with its transcript as annotated under NCBI Genbank accession number NM_005245.4.

Dedicated databases exist which annotate known disease-causing or associated mutations in human genes and proteins. By means of illustration, FAT1 mutations can be found in the Online Mendelian Inheritance in Man® (OMIM®, https://www.omim.org/) under accession number *600976, in the Cancer Genome Atlas (TCGA) Program's GDC data portal (https://portal.gdc.cancer.gov/) under accession number ENSG00000083857, or in the Catalogue of Somatic Mutations in Cancer (COSMIC, https://cancer.sanger.ac.uk/cosmic) under accession number COSG86438. The recent publication of the ICGC/TCGA Pan-Cancer Analysis of Whole Genomes Consortium (Nature 2020, vol. 578, 82-93) describes an integrative analysis of 2,658 whole-cancer genomes and their matching normal tissues across 38 tumor types; the associate resources are available via the data portal and visualisations at https://docs.icgc.org/pcawg/.

Databases, such as the above mentioned Cancer Genome Atlas Program's GDC data portal and the Catalogue of Somatic Mutations in Cancer (COSMIC, https://cancer.sanger.ac.uk/cosmic), also hold information on the potential functional effect of the FAT1 mutation. For example, it may be indicated in the database whether the mutation in the FAT1 gene is a missense mutation, a nonsense mutation or a frameshift mutation.

A person skilled in the art knows how to predict or determine whether a FAT1 mutation is a LOF mutation.

By means of examples, a FAT1 missense mutation typically results in the substitution of one amino acid for another in the amino acid sequence of the FAT1 protein. Depending on the location and type of the substitution, the amino acid substitution may for example have no effect, may render the FAT1 protein non-functional, may enhance the function of the FAT1 protein, or may endow the FAT1 protein with some novel activity, property or interaction. A disruptive in-frame mutation is a mutation that does not result in a frameshift as described elsewhere herein, but that disrupts the FAT1 status (e.g. FAT1 expression and/or function). For example, a disruptive in-frame mutation may be a FAT1 nonsense mutation. A FAT1 nonsense mutation typically results in a stop-gain, a stop-loss, a start-gain, or a start-loss. A stop-gain mutation typically results in the appearance of a stop codon in the amino sequence of the FAT1 protein, where previously there was a codon specifying an amino acid. The presence of a premature stop codon leads to the production of a C-terminally truncated version of the FAT1 protein. A stop-loss mutation is a mutation in the original termination codon, resulting in an abnormal extension of the FAT1 protein's carboxyl terminus. A start-gain mutation results in the appearance of a start codon upstream of the original start site. If the new start codon creates a frame shift as described elsewhere herein, the gene product may have a completely different amino acid sequence than the non-mutated gene product. If the new start codon is in frame with the sequence encoding FAT1, additional amino acids may be added to the amino terminus of the original FAT1 protein. A start-loss mutation is a mutation in the AUG start codon of the transcript, resulting in a reduction or complete elimination of the production of the FAT1 gene product. Accordingly, the person skilled in the art will understand that a FAT1 nonsense mutation typically results in a reduced or abolished FAT1 function. An insertion or deletion of one or more nucleotides in an exon of the FAT1 gene can result in a FAT1 frameshift mutation. Due to the triplet nature of codons in the genetic code, insertion or deletion of a X nucleic acids, wherein X is not 0 or a multiple of 3, can change the reading frame (i.e. the grouping of codons) resulting in a gene product wherein downstream of the frameshift mutation the gene product has a completely different amino acid sequence than the non-mutated gene product. A FAT1 frameshift mutation may also lead to a premature stop codon and a C-terminally truncated version of the protein. Accordingly, the person skilled in the art will understand that a FAT1 frameshift mutation typically results in a reduced or abolished FAT1 function. A mutation in an exon, intron or at an exon-intron boundary may alter the splicing of the FAT1 protein's pre-mRNA, leading for example to skipping of one or more exons or inclusion of one or more introns, with or without a shift in the reading frame. Such mutations are also referred to as splicing mutations. By means of example, retention of large segments of intronic DNA by the mRNA, or splicing out entire exons typically results in the production of a non-functional FAT1 protein, and hence, a reduced or abolished FAT1 function.

The potential effect of a FAT1 mutation, such as a FAT1 missense mutation, on expression or function of FAT1, can be predicted using SIFT (version 5.2.2, http://sift.jcvi.org/), PolyPhen-2 (version 2.2.2, http://genetics.bwh.harvard.edu/pph2/) and MEMo (e.g. as described in Ciriello et al., Mutual exclusivity analysis identifies oncogenic network modules, Genome Res, 2012, vol. 22(2): 398-406)) Furthermore, an alteration in the FAT1 expression and/or function caused by the one or more genetic alteration may be determined as described elsewhere herein.

In particular embodiments, said one or more FAT1 loss-of-function mutations are selected from the group consisting of a disruptive in-frame mutation, a missense mutation, a nonsense mutation (e.g. a stop-gain or a start-loss mutation), a frameshift mutation, a splicing mutation, and a combination thereof, preferably one or more FAT1 loss-of-function mutations are selected from the group consisting of a disruptive in-frame mutation, a missense mutation, a stop-gain mutation, and a combination thereof.

In particular embodiments, said one or more FAT1 loss-of-function mutations are selected from the group consisting of a missense mutation, a nonsense mutation, a frameshift mutation, a splicing mutation, and a combination thereof, preferably one or more FAT1 loss-of-function mutations are selected from the group consisting of a disruptive in-frame mutation, a missense mutation, a stop-gain mutation, and a combination thereof, and are predicted to cause a decreased FAT1 expression and/or function by prediction software (such as the SIFT, the PolyPhen-2 and/or the MEMo prediction software), preferably by at least three different prediction software, more preferably by all three of the SIFT, the PolyPhen-2 and the MEMo prediction software.

For example, said one ore more FAT1 loss-of-function mutations may comprise a missense mutation leading to the substitution of Thr at position 3284 of SEQ ID NO: 1 to Arg, wherein said missense mutation occurs in one or both of the FAT1 alleles, said one ore more FAT1 loss-of-function mutations may comprise a missense mutation leading to the substitution of Thr at position 1585 of SEQ ID NO: 1 to Met, wherein said missense mutation occurs in one or both of the FAT1 alleles; said one ore more FAT1 loss-of-function mutations may comprise a missense mutation leading to the substitution of Pro at position 1763 of SEQ ID NO: 1 to Leu, wherein said missense mutation occurs in one or both of the FAT1 alleles; said one ore more FAT1 loss-of-function mutations may comprise a stop-gain mutation leading to the replacement of the Arg at position 885 of SEQ ID NO: 1 by a stop codon, wherein said stop-gain mutation occurs in one or both of the FAT1 alleles; said one ore more FAT1 loss-of-function mutations may comprise a stop-gain mutation leading to the replacement of the Leu at position 148 of SEQ ID NO: 1 by a stop codon, wherein said stop-gain mutation occurs in both of the FAT1 alleles; said one ore more FAT1 loss-of-function mutations may comprise a stop-gain mutation leading to the replacement of the Tyr at position 372 of SEQ ID NO: 1 by a stop codon, wherein said stop-gain mutation occurs in one or both of the FAT1 alleles; said one ore more FAT1 loss-of-function mutations may comprise a stop-gain mutation leading to the replacement of the Gly at position 3179 of SEQ ID NO: 1 by a stop codon, wherein said stop-gain mutation occurs in both of the FAT1 alleles; and/or said one ore more FAT1 loss-of-function mutations may comprise a disruptive in-frame mutation leading to the deletion of the Val at position 3009 of SEQ ID NO: 1, wherein said disruptive in-frame mutation occurs in both of the FAT1 alleles.

Methods for determining the presence or absence of one or more FAT1 mutations are well known in the art and include, but are not limited to, fluorescence in situ hybridization (FISH) (e.g. mutation-specific FISH), genomic sequencing (e.g. direct genomic sequencing, Sanger sequencing, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell sequencing or the like), optionally in combination with polymerase-based nucleic acid amplification (e.g. PCR or the like). Sequencing may for example scrutinise the entire FAT1 gene, including non-transcribed sequences such as the promoter and any enhancer, and transcribed sequences such as exons, introns and UTRs; or may focus on transcribed sequences; or may focus on exons or coding sequences only (exome sequencing). Sequencing may also specifically focus on any known mutation hotspots in FAT1.

By means of examples, polymerase-based nucleic acid amplification, such as PCR, can be used to amplify the FAT1 gene sequence directly from a genomic DNA preparation from a biological sample comprising one or more neoplastic cells, such as a tumor biopsy or a liquid biopsy. The DNA sequence of the amplified sequences can then be determined using sequencing and mutations identified therefrom by comparing to a reference.

The phrase “polymerase-based nucleic acid amplification” as used herein generally encompasses any in vitro process for increasing the number of copies of a target nucleic acid region within a nucleic acid molecule, preferably within a DNA molecule, by the action of a nucleic acid polymerase, e.g., DNA polymerase. The process may encompass both linear and exponential amplification, and particularly preferably refers to exponential amplification. The process may particularly preferably refer to polymerase chain reaction (PCR). In PCR, target nucleic acid region within a nucleic acid molecule, especially within a DNA molecule, is amplified using thermostable DNA polymerase(s) and at least two amplification primers, one complementary to the (+)-strand at one end of the target sequence to be amplified and the other complementary to the (−)-strand at the other end of the target sequence. A reference to PCR as used herein encompasses modifications of the prototypic PCR, such as, e.g., high-fidelity PCR, hot-start PCR, touch-down PCR, nested PCR, multiplex PCR, quantitative PCR, quantitative real-time PCR, long-range PCR, RT-PCR, etc. (see, e.g., PCR Protocols: A Guide to Methods and Applications, eds. Innis et al., Academic Press, San Diego, 1990).

Primers or primer pairs for sequencing or amplification of the FAT1 gene may be designed using any method known in the art, for example by use of primer design tools, such as Primer3.

By means of further examples, the presence or absence of one or more FAT1 (LOF) mutations may be determined by contacting a nucleic acid sequence, preferably DNA, from the biological sample with a fluorescently-labelled probe that is capable of specifically hybridizing to the nucleic acid sequence comprising the one or more FAT1 genetic alterations, and detecting said hybridization.

Chromosomal aberrations include any irregularity or abnormality of chromosome distribution, number, structure, or arrangement. Such structural abnormalities may be a deletion of a fragment of the chromosome, a duplication of a fragment of the chromosome, a translocations of a fragment of one chromosome to another chromosome, an inversions of a fragment of the chromosome, an insertion of a fragment of a chromosome (e.g. such as a fragment which has been deleted from one chromosome and has been inserted into another chromosome), rings and isochromosomes. Methods for determining chromosomal aberrations are known in the art and may include metaphase cytogenetic (MC) analysis, fluorescence in situ hybridization (FISH), Comparative Genomic Hybridization (CGH), spectral karyotyping (SKY) and sequencing (e.g. Next Generation Sequencing (NGS) and single-cell sequencing).

FAT1 has at cytogenetic location 4q35.2 and molecular location from base pairs 186 587 789 to 186 726 696 on chromosome 4. Accordingly, any chromosomal aberration disturbing the chromosome 4q35.2 sequence, such as a deletion of chromosome 4q35.2 or a fragment thereof, or an insertion of a fragment of another chromosome into chromosome 4q35.2, may result in reduced or abolished expression or function of FAT1.

In particular embodiments, the methods as taught herein may encompass determining in a biological sample obtained from said subject the presence or absence of one or more chromosomal aberrations on chromosome 4, preferably on chromosome 4q35.2. In particular embodiments, the methods as taught herein may encompass determining in a biological sample obtained from said subject the deletion of chromosome 4 or a fragment thereof, preferably the deletion of chromosome 4q35.2.

A copy number aberration (CNA) is an acquired numerical change (i.e. increase or decrease of number of copies) of a chromosome or chromosomal segment in comparison with a reference genome. Genes located within an amplified chromosome region are typically overexpressed, while genes located in a region with chromosome losses are typically downregulated. Methods for determining chromosomal aberrations are known in the art and may include Comparative Genomic Hybridization (CGH) and sequencing (e.g. Next Generation Sequencing (NGS) and single-cell sequencing).

A reduced copy number of chromosome 4, and more specifically chromosome 4q35.2, may result in reduced or abolished expression or function of FAT1. Accordingly, in particular embodiments, the methods as taught herein may encompass determining in a biological sample obtained from said subject the presence or absence of a copy number variation on chromosome 4, preferably on chromosome 4q35.2. In more particular embodiments, the methods as taught herein may encompass determining in a biological sample obtained from said subject the presence or absence of a reduced copy number of chromosome 4, preferably of chromosome 4q35.2, compared to a reference genome. Epigenetic alterations, including DNA methylation, histone modifications and ATP-dependent chromatin-remodeling, can alter DNA accessibility and chromatin structure, which may result in an alteration of the expression and/or function of a gene product (e.g. increase or decrease, depending on where the alterations occur), without changing the nucleic acid sequence of the gene encoding the gene product as such. An epigenetic alteration resulting in silencing or downregulation of the gene, may also be referred to as epigenetic silencing.

Aberrant DNA hype methylation, which may be referred to as hypermethylation, is an epigenetic modification whereby the gene activity is controlled by adding methyl groups (CH₃) to specific cytosines of the DNA. In particular, methylation occurs m the cytosine of the CpG dinucleotides (CpG islands) which are concentrated in the promoter regions and introns in human genes. DNA methylation is typically associated with gene silencing as a result of blocking the DNA binding proteins that act as or recruit transcriptional activators or as a result of the recruitment of methyl-binding proteins, which recruit transcriptional corepressor complexes.

Typically, the methylation status is determined in suitable CpG islands which are often found in the promoter region of the gene(s). The term “methylation”, “methylation state” or “methylation status” refers to the presence or absence of 5-methylcytosine (“5-mCyt”) at one or a plurality of CpG dinucleotides within a DNA sequence. The methylation status of the FAT1 gene may be determined by any methods known in the art for determining DNA methylation, including, but not being limited to, bisulfite sequencing, pyrosequencing, methylation-specific PCR, PCR with high resolution meting or COLD-PCR for the detection of unmethylated islands as described in Kurdyukov S. and Bullock M., DNA methylation analysis: choosing the right method, Biology, 2016, 5(1):3.

Accordingly, in a certain embodiment, the methods as taught herein comprise determining the methylation state of genomic CpG sequences, wherein the genomic CpG sequences are located within the FAT1 gene. The person skilled in the art will understand that an increased methylation state (e.g. hypermethylation) of the genomic CpG sequences located within the FAT1 gene, such as CpG sequences within the promoter region of the FAT1 gene, compared to a reference, typically leads to a reduced or abolished expression or function of FAT1.

In a certain embodiment, the method as taught herein comprises determining the methylation state of the promoter region of the FAT1 gene.

Reference to a “promoter” or “promoter region” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. The promoter region may comprise a transcription start site, a binding site for RNA polymerase, general transcription factor binding sites (e.g. TATA box with sequence TATA or B recognition element with consensus sequence SSRCGCC (SEQ ID NO: 4) or RTDKKKK (SEQ ID NO: 5)) and optionally other elements or motifs. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene (hence, enhancers in introns and intergenic sequences are also a common occurrence).

Furthermore, the accessibility of large regions of DNA can also depend on its chromatin structure. There are two states of chromatin; euchromatin is open and amenable to transcription, whereas heterochromatin is a compact DNA-protein structure that cannot be transcribed. Chemical modifications to histones (e.g. methylation, acetylation and phosphorylation) cause conversion of DNA from a euchromatin state to a heterochromatin state and vice versa. For example, histone methylation may increase packing (i.e. decrease transcription) (e.g. H3K9me3 or H3K27me3) or decrease packing (i.e. increase transcription) (e.g. H3K4me2, H3K4me3, H3K36me3, or H3K79me), depending on the methylation site, while histone acetylation and phosphorylation typically decrease packing (i.e. increase transcription). DNA transcription may be regulated by post-translational chemical modifications to histone proteins. Accordingly, histone modification may result in an alteration of the expression and/or function of FAT1.

Histone modifications, including FAT7 allele-specific histone modifications, can be determined by any methods known in the art for determining histone modifications, including, but not being limited to chromatin immunoprecipitation (ChIP), ChIP-SAGE (Chromatin immunoprecipitation combined with serial analysis of gene expression), ChIP-Seq (Chromatin immunoprecipitation combined with high-throughput sequencing techniques) or ChIP in combination with in situ hybridization (ISH) and proximity ligation assay (PLA).

The person skilled in the art will understand that histone modifications converting the DNA of the FAT1 gene to a heterochromatin state typically result in a decreased or abolished expression and/or function of FAT1. Accordingly, in certain embodiments, the methods as taught herein comprise determining histone modifications within the FAT1 gene.

ATP-dependent chromatin-remodeling complexes utilize the energy from ATP hydrolysis to reorganize chromatin and, hence, regulate gene expression. Accordingly, ATP-dependent chromatin-remodeling may result in an alteration of the expression and/or function of FAT1. ATP-dependent chromatin-remodeling can be determined by any methods known in the art for determining histone modifications, including, but not being limited to ChiP. Accordingly, in a certain embodiment, the methods as taught herein comprise determining ATP-dependent chromatin-remodeling within the FAT1 gene. Reduced or abolished expression or function of FAT1 can also result from alterations in the FAT1 gene product, including FAT1 mRNA and the FAT1 protein.

Accordingly, in particular embodiments, the methods as taught herein may comprise determining in a biological sample obtained from said subject the presence or absence of an alteration in the FAT1 mRNA or FAT1 protein leading to reduced or abolished expression or function of FAT1.

By means of example, reduced or abolished expression or function of FAT1 may result from post-transcriptional modifications or co-transcriptional modifications (e.g. RNA modifications such as N⁶-methyladenosine modification), and/or co- or post-expression-type modifications as generally known in the art.

As indicated earlier, a mutated FAT1 gene may encode a mutated FAT1 protein, for example, when one amino acid is substituted by another. As used herein, the term “mutant” of a protein may in particular denote a form of the protein which differs from the protein in its amino acid sequence, wherein the mutant form is encoded by the same gene or locus as the protein, but wherein the nucleic acid sequence of that gene has been changed such as to encode the mutant form of the protein. As described elsewhere herein, sometimes, adjectives such as “unmodified”, “unchanged”, “original”, “starting” may be used in conjunction with the term “FAT1” to emphasise the distinction between FAT1 and its mutants. Alterations in the FAT1 amino acid sequence may be determined by methods known in the art for determining protein alterations, such as by mass spectrometry or using antibodies specifically recognizing a mutant FAT1.

The alterations in the FAT1 mRNA or FAT1 protein negatively influencing the FAT1 status may be caused by one or more FAT1 loss-of-function mutations. Hence, genetic or epigenetic modifications impinging on the expression or activity of FAT1 may be investigated as described above. In certain embodiments, the expression or function of FAT1 can also be determined as such.

Hence, in particular embodiments, the methods as taught herein comprise determining whether FAT1 expression is reduced or abolished in a biological sample obtained from said subject, such as compared to a reference. In other words, the methods as taught herein comprise determining whether the FAT1 mRNA level and/or the FAT1 protein level is reduced or abolished in a biological sample from said subject.

The terms “expression”, “expression level”, “quantity”, “amount” and “level”, are used interchangeably in this specification to refer to an absolute quantification of a molecule or an analyte in a sample, or to a relative quantification of a molecule or analyte in a sample, i.e., relative to another value such as relative to a reference value or range, such as a reference indicating a base-line expression of a marker in a given tissue. These values or ranges can be obtained from a single subject or from a group of subjects. An absolute quantity of a molecule or analyte in a sample may be advantageously expressed as weight or as molar amount, or more commonly as a concentration, e.g., weight per volume or mol per volume. A relative quantity of a molecule or analyte in a sample may be advantageously expressed as an increase or decrease or as a fold-increase or fold-decrease relative to the reference value or range. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

A reduced or abolished FAT1 expression (e.g. FAT1 mRNA level and/or the FAT1 protein level) may be determined using any methods known in the art for determining mRNA and/or protein levels.

In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridisation-based analysis, microarray expression analysis, digital gene expression profiling (DGE), RNA-in-situ hybridisation (RISH), Northern-blot analysis and the like; Polymerase Chain Reaction (PCR), Reverse Transcription PCR (RT-PCR), RT-quantitative PCR (qPCR), end-point PCR, digital PCR, digital droplet PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like.

In other examples, such methods may include methods for separating, detecting and/or quantifying markers at the protein level. Such methods are well known in the art and include, immunological assay methods, wherein the ability of an assay to separate, detect and/or quantify a peptide, polypeptide, or protein is conferred by specific binding between a separable, detectable and/or quantifiable binding agent such as an immunological binding agent (e.g. antibody) and the peptide, polypeptide, or protein. Immunological assay methods include without limitation immunohistochemistry, immunofluorescence, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc.

In other examples, such methods may include chromatography methods. The term “chromatography” encompasses methods for separating substances, such as chemical or biological substances, e.g., markers, such as preferably peptides, polypeptides, or proteins, referred to as such and vastly available in the art. In a preferred approach, chromatography refers to a process in which a mixture of substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilised metal affinity chromatography, and the like.

Further techniques for separating, detecting and/or quantifying markers, such as preferably peptides, polypeptides, or proteins, may be used, optionally in conjunction with any of the above described analysis methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In particular embodiments, the methods as taught herein comprise determining whether FAT1 function is reduced or abolished in a biological sample obtained from said subject.

The term “biological function” or “function” as used herein, is to be interpreted broadly and may generally encompass any one or more aspects of the biological function of the target at any level (e.g., molecular, cellular and/or physiological), such as without limitation any one or more aspects of its biochemical activity, signalling activity, interaction activity, receptor activity or structural activity (e.g., in or on a cell, cell population, tissue, organ, or organism, e.g., in a biological sample from a subject).

Present inventors found that Fat1 deletion that promotes the acquisition of a hybrid EMT state. Furthermore, present inventors found that the hybrid EMT signature in Fat1 mutants is mediated by the activation of YAP1 and Sox2, which regulates respectively the co-expression of mesenchymal and epithelial transcriptional programs in cancer cells. Moreover, the inventors found that Fat1 deletion activates CAMK2, which induced the phosphorylation of SRC/YES and CD44, which promote YAP nuclear translocation and the induction of EMT program including ZEB1 expression. CAMK2 activation also lead to the phosphorylation of EZH2 at Thr487, which inhibits its activity and lead to a decrease of the chromatin repressive mark at SOX2 regulatory regions, which lead to SOX2 upregulation, sustaining the expression of the epithelial program. In addition, FAT1 deletion also decreases the activation of EGFR/MEK pathway.

By means of examples and not limitation, a reduced or abolished FAT1 function may be determined by determining the expression of YAP1 or SOX9 or by determining the activation of the CAMK2 or EGFR/MEK pathway. Methods for determining expression levels of a gene product are described elsewhere herein. Methods for determining the activation of the CAMK2 are known in the art and include determining the phosphorylation of CAMK2 or a target thereof, such as SRC or CD44 by Western blot or flow cytometry. Methods for determining the expression level are described elsewhere herein. Methods for determining the activation of the EGFR/MEK pathway are known in the art and include determining the phosphorylation of EGFR or MEK by Western blot.

In further examples, any combinations of methods for determining the FAT1 status as discussed herein may be employed. The person skilled in the art will understand that the methods for determining the FAT1 status may depend on, inter alia, the type of a marker (e.g., peptide, polypeptide, protein, or nucleic acid), the type of the tested object (e.g., a cell, cell population, tissue, organ, or organism, e.g., the type of biological sample of a subject, e.g., whole blood, tissue biopsy), the expected abundance of the marker in the tested object, the type, robustness, sensitivity and/or specificity of the detection method used to detect the marker, etc., the marker may be measured directly in the tested object, or the tested object may be subjected to one or more processing steps aimed at achieving an adequate measurement of the marker.

In particular embodiments, the presence or absence of the genetic or epigenetic alteration or the expression or function of FAT1 is determined using a technique selected from the group consisting of nucleic acid analysis, immunological assay, functional assay, and a combination thereof.

In certain embodiments, the aforementioned methods and techniques may employ agent(s) capable of specifically binding FAT1, such as binding to the FAT1 gene or the FAT1 gene product (e.g. FAT1 mRNA or FAT1 protein).

Binding agents may be in various forms, e.g., lyophilized, free in solution, or immobilized on a solid phase. They may be, e.g., provided in a multi-well plate or as an array or microarray, or they may be packaged separately, individually, or in combination.

Binding agents as intended throughout this specification may include inter alia antibodies, antibody fragments, antibody-like protein scaffolds, aptamers, spiegelmers (L-aptamers), photoaptamers, proteins, peptides, peptidomimetics, nucleic acids such as oligonucleotides (e.g., hybridization probes or amplification or sequencing primers and primer pairs), small molecules, or combinations thereof.

The term “specifically bind” as used throughout this specification means that an agent (denoted herein also as “binding agent” or “specific-binding agent”) binds to one or more desired molecules or analytes (e.g., peptides, polypeptides, proteins, or nucleic acids) substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold, or at least about 1000-fold, or at least about 10⁴-fold, or at least about 10⁵-fold, or at least about 10⁶-fold or more greater, than its affinity for a non-target molecule, such as for a suitable control molecule (e.g., bovine serum albumin, casein).

Preferably, the specific binding agent may bind to its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹ or K_{A}≥1×10¹² M⁻¹, wherein K_{A}=[SBA_T]/[SBA][T], SBA denotes the specific-binding agent, T denotes the intended target. Determination of K_{A}≥can be carried out by methods known in the art, such as for example, using equilibrium dialysis and Scatchard plot analysis.

Antibodies suitable for determining the level of human FAT1 protein are known in the art and include anti-FAT1 antibody produced in rabbit from Sigma-Aldrich identified by catalogue number HPA023882, anti-FAT/FAT1 antibody from abcam identified by catalogue number ab190242, and FAT1 antibody (A304-403A) of ThermoFischer Scientific identified by catalogue number ii A304-403A.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

In certain embodiments, the agent may be a Nanobody®. The terms “Nanobody®” and “Nanobodies®” are trademarks of Ablynx NV (Belgium). The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “oligonucleotide” as used throughout this specification refers to a nucleic acid (including nucleic acid analogues and mimetics) oligomer or polymer as defined herein. Preferably, an oligonucleotide, such as more particularly an antisense oligonucleotide, is (substantially) single-stranded. Oligonucleotides as intended herein may be preferably between about 10 and about 100 nucleoside units (i.e., nucleotides or nucleotide analogues) in length, preferably between about 15 and about 50, more preferably between about 20 and about 40, also preferably between about 20 and about Oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, increased stability in the presence of nucleases and increased hybridization affinity, increased tolerance for mismatches, etc.

Nucleic acid binding agents, such as oligonucleotide binding agents, are typically at least partly antisense to a target nucleic acid of interest. The term “antisense” generally refers to an agent (e.g., an oligonucleotide) configured to specifically anneal with (hybridize to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein, such as hybridization probes or amplification or sequencing primers and primer pairs) may typically be capable of annealing with (hybridizing to) the respective target nucleic acid sequences at high stringency conditions, and capable of hybridizing specifically to the target under physiological conditions. The terms “complementary” or “complementarity” as used throughout this specification with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The reference to oligonucleotides may in particular but without limitation include hybridization probes and/or amplification primers and/or sequencing primers, etc., as commonly used in nucleic acid detection technologies.

The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

Binding agents as discussed herein may suitably comprise a detectable label. The term “label” refers to any atom, molecule, moiety or biomolecule that may be used to provide a detectable and preferably quantifiable read-out or property, and that may be attached to or made part of an entity of interest, such as a binding agent. Labels may be suitably detectable by for example mass spectrometric, spectroscopic, optical, colorimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes alone or in combination with moieties that may suppress or shift emission spectra by fluorescence resonance energy transfer (FRET).

In some embodiments, binding agents may be provided with a tag that permits detection with another agent (e.g., with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc.

The marker-binding agent conjugate may be associated with or attached to a detection agent to facilitate detection. Examples of detection agents include, but are not limited to, luminescent labels; colorimetric labels, such as dyes; fluorescent labels; or chemical labels, such as electroactive agents (e.g., ferrocyanide); enzymes; radioactive labels; or radiofrequency labels. The detection agent may be a particle. Examples of such particles include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles may be colloidal gold particles.

The FAT1 status in the biological sample of the subject diagnosed with a neoplastic disease may be determined in comparison to a reference value. For example, the reduced or abolished expression or function of FAT1 in the biological sample of the subject may refer to a relative quantity of FAT1 expression or function, i.e. the quantity of FAT1 expression or function in the biological sample of the subject compared with the quantity of FAT1 expression or function in a reference sample, e.g. of non-diseased tissue, such as non-diseased tissue of the same type as the tissue from which the neoplastic cells have originated, from the same subject or from one or more unrelated subject.

The presence of genetic alterations leading to reduced or abolished expression or function of FAT1 may be determined in reference to the FAT1 gene sequences present in healthy (non-neoplastic) tissues of the subject, such as in blood or saliva. Further, as many cancer-associated FAT1 mutations have been previously documented, and the effect (such as LOF effect) of many FAT1 mutations can be confidently predicted based on the nature of the mutations, in certain situations a reference may disposed with altogether, as the mutations can be simply detected, such as by sequencing, and ‘called’ as having the expected effect on FAT1.

For the epigenetic alterations leading to reduced or abolished expression or function of FAT1, the reference may be constituted by the epigenetic status in a healthy tissue, for example, a) in a tissue from a healthy subject that is of the same tissue type as the tissue that is afflicted by a pathology in a diseased subject, or b) in tissue from the diseased subject that is of the same tissue type as the afflicted tissue but is not afflicted by the pathology.

For the FAT1 expression or FAT1 function, the reference value may correspond to the expression or function of FAT1 in a healthy tissue, for example, a) in a tissue from a healthy subject that is of the same tissue type as the tissue that is afflicted by a pathology in a diseased subject, or b) in tissue from the diseased subject that is of the same tissue type as the afflicted tissue but is not afflicted by the pathology.

The reference values for the genetic or epigenetic alterations leading to reduced or abolished expression or function of FAT1, or for the FAT1 expression or FAT1 function may also represent one or more reference subjects who are sensitive or resistant to the treatment with the antineoplastic agent.

Reference values may be established according to known procedures, as already indicated above. For example, a reference value may be established in non-neoplastic cells of the same subject that suffers from the neoplastic disease, wherein the non-neoplastic cells are of the same cell type as the cells from which the neoplastic cells have originated, for example wherein neoplastic tissue is analysed with reference to non-neoplastic tissue of the same kind. A reference value can also be established in a tissue from a reference subject or individual or from a population of individuals, such as from a healthy subject or individual or from a population of healthy individuals. Such population may comprise without limitation 2 or more, 10 or more, 100 or more, or even several hundred or more individuals.

In certain embodiments, the methods as taught herein comprise:
determining in a biological sample of a tissue afflicted with a neoplastic disease the presence or absence of a genetic alteration leading to reduced or abolished expression or function of FAT1, wherein said determination is made with reference to a biological sample not afflicted by the neoplastic disease obtained from said subject or with reference to a FAT1 wild-type sequence, or
determining in a biological sample of a tissue afflicted with a neoplastic disease the presence or absence of a epigenetic alteration leading to reduced or abolished expression or function of FAT1, wherein said determination is made with reference to a biological sample of the same tissue type as the afflicted tissue but not afflicted by the neoplastic disease obtained from said subject, or
determining whether FAT1 expression or function is reduced or abolished in a biological sample of a tissue afflicted with a neoplastic disease, wherein said determination is made with reference to a biological sample of the same tissue type as the afflicted tissue but not afflicted by the neoplastic disease obtained from said subject.

In certain embodiments, the methods as taught herein comprise:
determining in a biological sample of a tissue afflicted by the neoplastic disease obtained from said subject diagnosed with said neoplastic disease the presence or absence of a genetic alteration leading to reduced or abolished expression or function of FAT1, wherein said determination is made with reference to a biological sample obtained from one or more healthy subjects or with reference to a FAT1 wild-type sequence, or
determining in a biological sample of a tissue afflicted by the neoplastic disease obtained from said subject diagnosed with said neoplastic disease the presence or absence of a epigenetic alteration leading to reduced or abolished expression or function of FAT1, wherein said determination is made with reference to a biological sample obtained from one or more healthy subjects that is of the same tissue type as the tissue that is afflicted by the neoplastic disease, or
determining whether FAT1 expression or function is reduced or abolished in a biological sample of a tissue afflicted by the neoplastic disease obtained from said subject diagnosed with said neoplastic disease, wherein said determination is made with reference to a biological sample obtained from one or more healthy subjects that is of the same tissue type as the tissue that is afflicted by the neoplastic disease.

In particular embodiments, the methods as taught herein comprise
(a) determining the expression or function of FAT1 in the biological sample obtained from the subject;
(b) comparing the expression or function of FAT1 as determined in (a) with a reference value representing the expression or function of FAT1 in a healthy tissue of the same type;
(c) finding a deviation or no deviation of the expression or function of FAT1 as determined in (a) from said reference value;
(d) attributing said finding of deviation or no deviation to a particular determination of sensitivity or resistance to treatment with the antineoplastic agent in the subject.

The healthy tissue may be, for example, a) a tissue from a healthy subject that is of the same tissue type as the tissue that is afflicted by a pathology in a diseased subject, orb) a tissue from the diseased subject that is of the same tissue type as the afflicted tissue but is not afflicted by the pathology.

Such comparison may generally include any means to determine the presence or absence of at least one difference or deviation and optionally of the size of such difference or deviation between values being compared. A comparison may include a visual inspection, an arithmetical or statistical comparison of measurements. Such statistical comparisons include, but are not limited to, applying a rule.

A “deviation” of a first value from a second value may generally encompass any direction (e.g., decrease: first value>second value; or increase: first value<second value) and any extent of alteration.

The observed deviation may be a decrease of the expression or function of FAT1 in the biological sample obtained from the subject compared to the reference value. For example, a deviation or decrease may encompass a decrease of a first value of the expression or function of FAT1 by, without limitation, at least about 10%, e.g., of at least about 20%, of at least about 30%, e.g., of at least about 40%, of at least about 50%, e.g., of at least about 60%, of at least about 70%, e.g., of at least about 80%, of at least about 90%, e.g., of at least about 95%, such as of at least about 96%, 97%, 98%, 99% or even of 100%, preferably at least about 70%, more preferably at least about 80%, even more preferably at least about 90%, relative to a second value of the expression or function of FAT1 with which a comparison is being made.

Preferably, a deviation or reduction may refer to a statistically significant observed alteration or reduction in the expression or function of FAT1. For example, a deviation or reduction may refer to an observed alteration or reduction, which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation or reduction may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

By extensive research, the present inventors have found that subject that show a decrease or total loss of FAT1 expression or function are resistant to treatment with an EGFR inhibitor and/or a MEK inhibitor, whereas subjects that show a decrease or total loss of FAT1 expression or function are sensitive to treatment with a CAMK inhibitor and/or a SRC kinase inhibitor.

Accordingly, in particular embodiments, if said antineoplastic agent is an antineoplastic agent selected from the group consisting of an EGFR inhibitor, a MEK inhibitor, and a combination thereof,
the determination in a biological sample obtained from said subject of the presence of the genetic or epigenetic alteration leading to reduced or abolished expression or function of FAT1; or
the determination of reduced or abolished FAT1 expression or function in a biological sample obtained from said subject,
   indicates that said subject is resistant (or unresponsive or insusceptible) to treatment with said antineoplastic agent.

In particular embodiments, if said antineoplastic agent is an antineoplastic agent selected from the group consisting of an EGFR inhibitor, a MEK inhibitor, and a combination thereof,
the determination in a biological sample obtained from said subject of the absence of the genetic or epigenetic alteration leading to reduced or abolished expression or function of FAT1; or
the determination of an unchanged or increased FAT1 expression or function in a biological sample obtained from said subject,
   indicates that said subject is sensitive (or responsive or susceptible) to treatment with said antineoplastic agent.

In certain embodiments of the methods as taught herein, wherein said antineoplastic agent is an antineoplastic agent selected from the group consisting of an EGFR inhibitor, a MEK inhibitor, and a combination thereof, the reference value represents the expression or function of FAT1 in a healthy tissue, and wherein:
(i) a reduced or abolished expression or function of FAT1 as determined in (a) compared with the reference value indicates that the subject will be resistant to treatment with the antineoplastic agent, or
(ii) the same or an increased expression or function of FAT1 as determined in (a) compared with the reference value indicates that the subject will be sensitive to treatment with the antineoplastic agent.

In particular embodiments if said antineoplastic agent is an antineoplastic agent selected from the group consisting of a CAMK inhibitor, a SRC kinase inhibitor, and a combination thereof,
the determination in a biological sample obtained from said subject of the presence of the genetic or epigenetic alteration leading to reduced or abolished expression or function of FAT1; or
the determination of reduced or abolished FAT1 expression or function in a biological sample obtained from said subject,
   indicates that said subject is sensitive (or responsive or susceptible) to treatment with said antineoplastic agent.

In particular embodiments, if said antineoplastic agent is an antineoplastic agent selected from the group consisting of a CAMK inhibitor, a SRC kinase inhibitor, and a combination thereof,
the determination in a biological sample obtained from said subject of the absence of the genetic or epigenetic alteration leading to reduced or abolished expression or function of FAT1; or
the determination of an unchanged or increased FAT1 expression or function in a biological sample obtained from said subject,
   indicates that said subject is resistant (or unresponsive or insusceptible) to treatment with said antineoplastic agent.

In certain embodiments of the methods as taught herein, wherein said antineoplastic agent is an antineoplastic agent selected from the group consisting of a CAMK inhibitor, a SRC kinase inhibitor, and a combination thereof, the reference value represents the expression or function of FAT1 in a healthy tissue, and wherein:
(i) a reduced or abolished expression or function of FAT1 as determined in (a) compared with the reference value indicates that the subject will be sensitive to treatment with the antineoplastic agent, or
(ii) the same or an increased expression or function of FAT1 as determined in (a) compared with the reference value indicates that the subject will be resistant to treatment with the antineoplastic agent.

The “antineoplastic agent” as used herein refers to a substance or composition capable of the alleviation or measurable lessening of one or more symptoms or measurable markers of a neoplastic disease. As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, fungi, plants, or animal cells or tissues. Preferred though non-limiting “agents” include nucleic acids, oligonucleotides, ribozymes, peptides, polypeptides, proteins, peptidomimetics, antibodies, antibody fragments, antibody-like protein scaffolds, aptamers, photoaptamers, spiegelmers, chemical substances, preferably organic molecules, more preferably small organic molecules, lipids, carbohydrates, polysaccharides, etc., and any combinations thereof. Depending on the context, the term “agent” may denote a “therapeutic agent” or “drug”, useful for or used in the treatment, cure, prevention, or diagnosis of a disease.

The term “inhibitor”, “inhibit”, or “inhibiting” as used herein is intended to be synonymous with terms such as “decrease”, “reduce”, “diminish”, “interfere”, “disrupt”, or “disturb”, and denotes a qualitative or quantitative decrease the expression and/or function of a protein, such as EGFR, a member of the MEK family of kinases, a member of the CAMK family of kinases or a member of the SRC family of kinases, that is being interfered with. The term encompasses any extent of such interference. For example, the interference may encompass a decrease (in particular statistically significant decrease) of at least about 25%, e.g., of at least about 30%, of at least about 35%, e.g., of at least about 40%, of at least about 50%, e.g., of at least about 60%, of at least about 70%, e.g., of at least about 80%, of at least about 90%, e.g., of at least about 95%, such as of at least about 96%, 97%, 98%, 99% or even of 100%, compared to a reference situation without said interference. A decrease in the expression and/or function of a protein, such as EGFR, a member of the MEK family of kinases, a member of the CAMK family of kinases or a member of the SRC family of kinases, may be determined as described elsewhere herein. By means of an example and not limitation, reference to the function of a protein, such as EGFR, a member of the MEK family of kinases, a member of the CAMK family of kinases or a member of the SRC family of kinases, may particularly denote the protein kinase activity of the polypeptide, as described elsewhere herein.

In particular embodiments, the inhibitor as disclosed herein comprises or is selected from a group consisting of a chemical substance, a protein, a polypeptide, a peptide, an antibody, an antibody fragment, an antibody-like protein scaffold, an aptamer, a photoaptamer, a spiegelmer, a peptidomimetic, a gene-editing system, a nucleic acid (including siRNA and shRNA), and combinations thereof. The structure of such classes of substances have already been discussed elsewhere in the specification.

In particular embodiments, the inhibitor is a protein kinase inhibitor.

The term “protein kinase inhibitor” as used herein refers to an agent that can decrease the enzymatic activity of a protein kinase, such as by at least 25%, at least 50%, at least 75%, at least 80%, at least 90%, at least 95%, such as 96%, 97% or 98%, or at least 99%, up to and including a 100% decrease, compared to the baseline protein kinase activity. Protein kinases are kinase enzymes that can modify other proteins by chemically adding one or more phosphate groups to them in a process called phosphorylation, which can turn a protein on or off and therefore affect its level of activity and function. Accordingly, the kinase inhibitor can decrease or eliminate the phosphorylation of one or more target proteins. Methods and means for determining the protein kinase activity are known in the art, and include assays based on detecting formation or a phosphorylated product or based on measuring the amount of ATP (phosphate donor) used or ADP formed.

EGFR, also known as ErbB-1 or human EGFR related (HER) 1, is a transmembrane protein that is a member of the ErbB family of tyrosine kinase receptors. Abnormal activation of EGFR is associated with tumor growth and progression and interruption of EGFR signaling can prevent the growth of EGFR-expressing tumors.

In particular embodiments, EGFR is human EGFR. Exemplary human EGFR protein sequence may be as annotated under U.S. government's National Center for Biotechnology Information (NCBI) Genbank (http://www.ncbi.nlm.nih.gov/) accession number NP_005219.2 (isoform a precursor), or Swissprot/Uniprot (http://www.uniprot.org/) accession number P00533.2. Exemplary human EGFR mRNA (cDNA) sequence may be as annotated under NCBI Genbank accession number NM_005228.5 (transcript variant 1).

Non-limiting examples of EGFR inhibitors include afatinib (e.g. Gilotrif™), lapatinib (e.g. Tykerb™ or Tyverb™), neratinib (e.g. Nerlynx™), gefitinib (Iressa™), erlotinib (Tarceva™), cetuximab (Erbitux™), osimertinib (Tagrisso™), panitumumab (Vectibix™), neratinib (Nerlynx™), vandetanib (Caprelsa™), necitumumab (Portrazza™), dacomitinib (Vizimpro™), and any combination thereof.

In particular embodiments, the EGFR inhibitor is a tyrosine kinase inhibitor (TKI). A TKI may bind the TK domain of the EGFR and stop the activity of the EGFR. Non-limiting examples of EGFR TKI include afatinib (e.g. Gilotrif™), lapatinib (e.g. Tykerb™ or Tyverb™), neratinib (e.g. Nerlynx™), and any combination thereof.

In particular embodiments, the EGFR inhibitor decreases the enzymatic activity of EGFR by at least 25%, at least 50%, at least 75%, at least 80%, at least 90%, at least 95%, such as 96%, 97% or 98%, or at least 99%, up to and including a 100% decrease, compared to the baseline EGFR enzymatic activity. Methods for measuring EGFR enzymatic activity are known in the art and include EGFR kinase activity assays, for example, Promega's EGFR Kinase Enzyme System with catalogue number V3831.

In particular embodiments, the EGFR inhibitor is a monoclonal antibody. Monoclonal antibodies targeting EGFR may bind the extracellular component of the EGFR and prevent the EGF from binding to the EGFR, thereby preventing cell division. Non-limiting examples of EGFR inhibiting monoclonal antobidiesinclude cetuximab (Erbitux™), panitumumab (Vectibix™), necitumumab (Portrazza™), and any combination thereof.

In particular embodiments, the EGFR inhibitor inhibits both the EGFR kinase and the human epidermal growth factor receptor 2 (HER2)/neu kinase. Non-limiting examples of inhibitors that inhibit both EGFR and HER2/neu include afatinib, lapatinib and neratinib.

In particular embodiments, said EGFR inhibitor is afatinib.

MEK or mitogen-activated protein kinase kinase is a family of dual specificity threonine/tyrosine kinases which activate the extracellular signal-regulated kinases by phosphorylation of threonine and tyrosine residues. MEK1 and MEK2 are the prototype members of MEK family proteins.

In particular embodiments, MEK1 is human MEK1. Exemplary human MEK1 protein sequence may be as annotated under U.S. government's National Center for Biotechnology Information (NCBI) Genbank (http://www.ncbi.nlm.nih.gov/) accession number NP_002746.1, or Swissprot/Uniprot (http://www.uniprot.org/) accession number Q02750.2. Exemplary human MEK1 mRNA (cDNA) sequence may be as annotated under NCBI Genbank accession number NM_002755.4.

In particular embodiments, MEK2 is human MEK2. Exemplary human MEK2 protein sequence may be as annotated under U.S. government's National Center for Biotechnology Information (NCBI) Genbank (http://www.ncbi.nlm.nih.gov/) accession number NP_109587.1, or Swissprot/Uniprot (http://www.uniprot.org/) accession number P36507.1. Exemplary human MEK2 mRNA (cDNA) sequence may be as annotated under NCBI Genbank accession number NM_030662.4.

Non-limiting examples of MEK inhibitors include trametinib (Mekinist™), selumetinib (Koselugo™), cobimetinib (Cotellic™), refametinib, pimasertib, PD0325901, MEK162, AZD8330, R04987655, R05126766, WX-554, E6201, GDC-0623, TAK-733, binimetinib, and any combination thereof.

In particular embodiments, the MEK inhibitor is a MEK inhibitor inhibiting MEK1 but not MEK2, a MEK inhibitor inhibiting MEK2 but not MEK1 or a MEK inhibitor inhibiting both MEK1 and MEK2.

In particular embodiments, the MEK inhibitor inhibits both MEK1 and MEK2. Non-limiting examples of MEK inhibitors inhibiting both MEK1 and MEK2 include trametinib, selumetinib, refametinib, pimasertib, PD0325901, MEK162, AZD8330, R04987655, R05126766, WX-554, E6201, TAK-733, binimetinib, and any combination thereof.

In particular embodiments, the MEK inhibitor decreases the enzymatic activity of MEK1 and MEK2 by at least 25%, at least 50%, at least 75%, at least 80%, at least 90%, at least 95%, such as 96%, 97% or 98%, or at least 99%, up to and including a 100% decrease, compared to the baseline MEK1 and MEK2 enzymatic activity. Methods for measuring MEK1 and MEK2 enzymatic activity are known in the art and include MEK1 and MEK2 kinase activity assays, for example, Promega's MEK1 Kinase Enzyme System with catalogue number VA7216.

In particular embodiments, said MEK inhibitor is trametinib.

CAMK, also known as CamK, is a serine/threonine protein kinase that becomes activated by increases in the concentrations of intracellular calcium ions and calmodulin. Members of the CAMK family include CAMKI (e.g. CAMKIalpha, CAMKI beta, CAMKI delta, CAMKI gamma), CAMKII (e.g. CAMKIIalpha, CAMKIIbeta, CAMKIIdelta, CAMKIIgamma), CAMKIII, CAMKIV, CAMKV CaM kinase like vesicle associated, SCAMK and Ca²⁺/calmodulin-dependent protein kinase kinase (e.g. CAMKK1 or CAMKK2).

Non-limiting examples of CAMK inhibitors include KN-93 (i.e. CAMKII inhibitor), KN-93 phosphate (i.e. CAMKII inhibitor), autocamtide-3 inhibitor (AC3-I)/autocamtide-2 inhibitor protein (AIP) (i.e. CAMKII inhibitor), CaMKIIN (i.e. CAMKII inhibitor), NH125 (i.e. CAMKIII inhibitor), STO-609 (i.e. CAMKKalpha and CAMKKbeta inhibitor), KN-62 (i.e. CAMKII inhibitor), and any combination thereof.

In particular embodiments, said CAMK inhibitor is a CAMKII inhibitor.

CAMKII has four isoforms (alpha, beta, gamma and delta). Each isoform is encoded by separate genes. CAMKII is a complex of 8 to 12 similar homologous subunits held together by interactions between a C-terminal association domain and arranged in a pinwheel-like structure.

In particular embodiments, CAMKII is human CAMKII. Exemplary human CAMKII subunit beta protein sequence may be as annotated under U.S. government's National Center for Biotechnology Information (NCBI) Genbank (http://www.ncbi.nlm.nih.gov/) accession number NP_001211.3 (isoform 1), or Swissprot/Uniprot (http://www.uniprot.org/) accession number Q13554.3. Exemplary human CAMKII subunit beta mRNA (cDNA) sequence may be as annotated under NCBI Genbank accession number NM_001220.5 (transcript variant 1).

In particular embodiments, the CAMKII inhibitor decreases the enzymatic activity of CAMKII by at least 25%, at least 50%, at least 75%, at least 80%, at least 90%, at least 95%, such as 96%, 97% or 98%, or at least 99%, up to and including a 100% decrease, compared to the baseline CAMKII enzymatic activity. Methods for measuring CAMKII enzymatic activity are known in the art and include CAMKII kinase activity assays, for example, Promega's CAMK2alpha Kinase Enzyme System with catalogue number V4018.

Non-limiting examples of CAMKII inhibitors include KN-93, KN-93 phosphate, autocamtide-3 inhibitor (AC3-I)/autocamtide-2 inhibitor protein (AIP), CaMKIIN, KN-62, and any combination thereof.

In particular embodiments, said CAMK inhibitor is KN93

Src family of kinases is a family of non-receptor tyrosine kinases. Members of the Src family of kinases include c-Src, Yes, Fyn, Fgr, Lck, Hck, Blk, Lyn and Frk. In particular embodiments, the Src family kinases are human Src family kinases with family members human c-Src, human Yes, human Fyn, human Fgr, human Lck, human Hck, human Blk, human Lyn and human Frk.

In particular embodiments, the SRC inhibitor inhibits at least two, at least three, at least four, at least five, at least six, at least seven, at least eight, preferably all nine, family members of the Src family of kinases selected from the group consisting of Yes, Fyn, Fgr, Lck, Hck, Blk, Lyn and Frk.

In particular embodiments, the SRC inhibitor inhibits c-Src and at least one, at least two, at least three, at least four, at least five, at least six, at least seven, preferably all eight family members of the Src family of kinases selected from the group consisting of c-Src, Yes, Fyn, Fgr, Lck, Hck, Blk, Lyn and Frk.

In particular embodiments, the SRC inhibitor decreases the enzymatic activity of at least one, such as at least two, at least three, at least four, at least five, at least six, at least seven, preferably all eight, kinases of the Src family of kinases selected from the group consisting of Yes, Fyn, Fgr, Lck, Hck, Blk, Lyn and Frk by at least 25%, at least 50%, at least 75%, at least 80%, at least 90%, at least 95%, such as 96%, 97% or 98%, or at least 99%, up to and including a 100% decrease, compared to the baseline enzymatic activity of the kinase(s) of the Src family of kinases.

Non-limiting examples of SRC inhibitors inhibiting all Src family of kinases selected from the group consisting of c-Src, Yes, Fyn, Fgr, Lck, Hck, Blk, Lyn and Frk include saracatinib (AZD-0530), dasatinib, KX2-391 and bosutinib (Bosulif™), and any combination thereof.

In particular embodiments, the SRC inhibitor inhibits at least one, preferably all, kinase of the Src family of kinases selected from the group consisting of Src, Yes, Fyn, Fgr, Lck, Hck, Blk, Lyn and Frk, and further also inhibits the BCR-ABL kinase.

Non-limiting examples of SRC inhibitors inhibiting at least one kinase of the Src family of kinases selected from the group consisting of Src, Yes, Fyn, Fgr, Lck, Hck, Blk, Lyn and Frk, and BCR-ALB include saracatinib (AZD-0530), dasatinib (Spyrel™), bosutinib (Bosulif™).

In particular embodiments, said SRC inhibitor is saracatinib or dasatinib.

The protein, polypeptide, or peptide antineoplastic agents may also include non-naturally encoded amino acids. A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. The protein, polypeptide, or peptide antineoplastic agents may also include un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

The nucleic acid antineoplastic agents may comprise modified nucleobases include without limitation pyrimidines, 6-azapyrimidines and N-2, N-6 and 0-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-0,4′-C-alkylene-linked, e.g., 2′-0,4′-C-methylene-linked or 2′-0,4′-C-ethylene-linked sugars such as ribose; 2′-fluoro-arabinose, etc.).

The term “gene editing system” or “genome editing system” as used herein refers to a tool to induce one or more nucleic acid modifications, such as DNA or RNA modifications, into a specific DNA or RNA sequence within a cell. Targeted genome modification is a powerful tool for genetic manipulation of cells and organisms, including mammals. Genome modification or gene editing, including insertion, deletion or replacement of DNA in the genome, can be carried out using a variety of known gene editing systems. Gene editing systems typically make use of an agent capable of inducing a nucleic acid modification. In certain embodiments, the agent capable of inducing a nucleic acid modification may be a (endo)nuclease or a variant thereof having altered or modified activity. (endo)Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through nonhomologous end-joining or homologous recombination, resulting in targeted mutations. In certain embodiments, said (endo)nuclease may be RNA-guided. In certain embodiments, said (endo)nuclease can be engineered nuclease such as a Clustered Regularly Interspaced Short Palindromic Repeats (CRISPR) associated (Cas) (endo)nuclease, such as Cas9, Cpf1, or C2c2, a (zinc finger nuclease (ZFN), a transcription factor-like effector nuclease (TALEN), a meganuclease, or modifications thereof. Methods for using TALEN technology, Zinc Finger technology and CRISPR/Cas technology are known by the skilled person.

The antineoplastic agent as disclosed herein may be an expressible molecule such as an antibody or a fragment or derivative thereof, a protein or polypeptide, a peptide, a nucleic acid, an antisense agent or an RNAi agent, it shall be understood that the agent itself may be introduced to a subject or may be introduced by means of a recombinant nucleic acid comprising a sequence encoding the agonist operably linked to one or more regulatory sequences allowing for expression of said sequence encoding the agent (e.g., gene therapy or cell therapy).

The term “neoplastic disease” generally refers to any disease or disorder characterized by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

In certain embodiments of the methods or uses as taught herein, the neoplastic disease may be a tumor or may be characterized by the presence of a tumor.

As used herein, the terms “tumor”, “solid tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

In particular embodiments said neoplastic disease is of epithelial, mesenchymal or melanocyte origin, preferably of epithelial origin.

In certain embodiments of the methods or uses as taught herein, the neoplastic disease may be cancer.

As used herein, the term “cancer” refers to a malignant neoplasm characterized by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, sarcoma, lymphoma, blastoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, esophageal cancer, thyroid cancer, or hematological cancer.

In certain embodiments of the methods or uses as taught herein, the neoplastic disease may be carcinoma, sarcoma or a solid tumor.

In certain embodiments of the methods or uses as taught herein, the neoplastic disease may be carcinoma.

In particular embodiments the neoplastic disease is a carcinoma, preferably a carcinoma originated from epithelial tissue selected from the group consisting of skin, lung, intestine, colon, breast, bladder, head and neck (including lips, oral cavity, salivary glands, nasal cavity, nasopharynx, paranasal sinuses, pharynx, throat, larynx, and associated structures), esophagus, thyroid, kidney, liver, pancreas, bladder, penis, testes, prostate, vagina, cervix, anus, and any combination thereof.

In certain embodiments of the methods or uses as taught herein, the neoplastic disease may be adenocarcinoma, squamous cell carcinoma (SCC), adenosquamous carcinoma, anaplastic carcinoma, large cell carcinoma, or small cell carcinoma. In certain embodiments of the methods or uses as taught herein, the neoplastic disease may be squamous cell carcinoma. For instance, the neoplastic disease may be skin squamous cell carcinoma.

In particular embodiments said neoplastic disease is a SCC, preferably a SCC of the skin, SCC of the head and neck, SCC of the oesophagus or SCC of the lung.

The term “carcinoma” refers to a category of types of cancer that develop from epithelial cells. For example, a carcinoma is a cancer that begins in a tissue that lines the inner or outer surfaces of the body, and that arises from cells originating in the endodermal, mesodermal or ectodermal germ layer during embryogenesis.

In certain embodiments of the methods or uses as taught herein, the neoplastic disease may be sarcoma. In certain embodiments of the methods or uses as taught herein, the neoplastic disease may be bone sarcoma or soft tissue sarcoma.

The term “sarcoma” refers to a category of types of cancer that arise from transformed cells of mesenchymal (connective tissue) origin. Connective tissue includes bone, cartilage, fat, vascular, or hematopoietic tissues, and sarcomas can arise in any of these types of tissues.

Subtypes of bone sarcoma include osteosarcoma, chondrosarcoma, poorly differentiated round/spindle cell tumors (includes Ewing sarcoma), hemangioendothelioma, angiosarcoma, fibrosarcoma, myofibrosarcoma, chordoma, adamantinoma.

Subtypes of soft tissue sarcoma include liposarcoma (includes the following varieties: well-differentiated, not otherwise specified, de-differentiated, myxoid/round cell, and pleomorphic); atypical lipomatous tumor; dermatofibrosarcoma protuberans (includes fibrosarcomatous and pigmented varieties); malignant solitary fibrous tumor; inflammatory myofibroblastic tumor; low-grade myofibroblastic sarcoma; fibrosarcoma (includes adult and sclerosing epithelioid varieties); myxofibrosarcoma (formerly myxoid malignant fibrous histiocytoma); low-grade fibromyxoid sarcoma; giant cell tumor of soft tissues; leiomyosarcoma; malignant glomus tumor; rhabdomyosarcoma (includes the following varieties: embryonal, alveolar, pleomorphic, and spindle cell/sclerosing); hemangioendothelioma (includes the following varieties: retiform, pseudomyogenic, and epithelioid); angiosarcoma of soft tissue; extraskeletal osteosarcoma; malignant gastrointestinal stromal tumor (GIST); malignant peripheral nerve sheath tumor (includes epithelioid variety); malignant Triton tumor; malignant granular cell tumor; malignant ossifying fibromyxoid tumor; stromal sarcoma not otherwise specified; myoepithelial carcinoma; malignant phosphaturic mesenchymal tumor; synovial sarcoma (includes the following varieties: spindle cell, biphasic, and not otherwise specified); epithelioid sarcoma; alveolar soft part sarcoma; clear cell sarcoma of soft tissue; extraskeletal myxoid chondrosarcoma; extraskeletal Ewing sarcoma; desmoplastic small round cell tumor; extrarenal rhabdoid tumor; perivascular epithelioid cell tumor, not otherwise specified; intimal sarcoma; undifferentiated spindle cell sarcoma; undifferentiated pleomorphic sarcoma; undifferentiated round cell sarcoma; undifferentiated epithelioid sarcoma; undifferentiated sarcoma, not otherwise specified.

In certain embodiments of the methods or uses as taught herein, the neoplastic disease may be a (solid) tumor.

The terms “sample” or “biological sample” as used throughout this specification include any biological specimen obtained (isolated, removed) from a subject. Samples may include without limitation organ tissue (e.g., primary or metastatic tumor tissue), whole blood, plasma, serum, whole blood cells, red blood cells, white blood cells (e.g., peripheral blood mononuclear cells), saliva, urine, stool (feces), tears, sweat, sebum, nipple aspirate, ductal lavage, tumor exudates, synovial fluid, cerebrospinal fluid, lymph, fine needle aspirate, amniotic fluid, any other bodily fluid, exudate or secretory fluid, cell lysates, cellular secretion products, inflammation fluid, semen and vaginal secretions. Preferably, a sample may be readily obtainable by non-invasive or minimally invasive methods, such as blood collection (‘liquid biopsy’), urine collection, feces collection, tissue (e.g., tumor tissue) biopsy or fine-needle aspiration, allowing the provision, removal, and/or isolation of the sample from a subject. The term “tissue” as used herein encompasses all types of cells of the body including cells of organs but also including blood and other body fluids recited above. The tissue may be healthy or affected by pathological alterations, e.g., tumor tissue.

The biological sample may be any sample in which the presence or absence of one or more FAT1 mutations can be determined. Particularly useful samples are those known to comprise, or expected or predicted to comprise, or known to potentially comprise, or expected or predicted to potentially comprise neoplastic cells.

A sample as intended herein contains genetic material of a subject. Hence, particularly useful samples are those known to comprise or expected or predicted to comprise genetic material of the subject. Genetic material in the present context encompasses any nucleic acid molecule or molecules in which the structure or sequence of the subject's FAT1 gene can be evaluated, and may particularly encompass subject's nuclear deoxyribonucleic acid (DNA), i.e., subject's nuclear genomic DNA.

In particular embodiments said biological sample obtained from said subject comprises neoplastic cells, preferably wherein said biological sample obtained from said subject is a tumor biopsy or a liquid biopsy.

The term “biopsy” generally denotes a sample of cells or tissues removed from a living subject for examination. The biopsy may be an excisional biopsy (i.e., when an entire lump or suspicious area is removed), an incisional biopsy or core biopsy (i.e., when only a sample of tissue is removed with preservation of the histological architecture of the tissue's cells), or a needle aspiration biopsy (i.e., when a sample of tissue or fluid is removed with a needle in such a way that cells are removed without preserving the histological architecture of the tissue cells).

The sample can be subjected to a variety of well-known post-collection preparative and storage techniques (e.g. fixation, storage, freezing, lysis, homogenization, DNA or RNA extraction, ultrafiltration, concentration, evaporation, centrifugation, etc.) prior to determining the presence or absence of the one or more FAT1 mutations in the sample.

Any suitable weight or volume of a sample may be removed from a subject for analysis. Without limitation, a liquid sample may have a volume between 0.1 ml and 1 ml such as 0.5 ml, or between 1 ml and 20 ml, such as 5 ml, 7.5 ml, 10 ml, 15 ml or 20 ml. A solid sample may have a weight of between 0.1 g and 20 g, such as 0.5 g, 1 g, 5 g, 7.5 g, 10 g, 15 g or 20 g.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain embodiments, the subject is a human subject. The term does not denote a particular age or sex. Thus, adult and new-born subjects, as well as fetuses, whether male or female, are intended to be covered. Examples of subjects include humans, dogs, cats, cows, goats, and mice.

Suitable subjects may include without limitation subjects presenting to a physician for a screening for a neoplastic disease, subjects presenting to a physician with symptoms and signs indicative of a neoplastic disease, subjects diagnosed with a neoplastic disease, subjects who have received anti-cancer therapy, subjects undergoing anti-cancer treatment, and subjects having a neoplastic disease in remission. Methods for diagnosing neoplastic diseases are known in the art and include microscopic analysis of a sample (biopsy) of the affected area of the tissue or organ, such as a skin biopsy.

In particular embodiments, said subject has not received an anti-cancer therapy prior to determining the sensitivity of said subject to said antineoplastic agent selected from the group consisting of an EGFR inhibitor, a MEK inhibitor, a CAMK inhibitor, and a SRC kinase inhibitor.

As used throughout this specification, the terms “therapy” or “treatment” refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. The terms “anti-cancer therapy” or “anti-cancer treatment” broadly refer to the alleviation or measurable lessening of one or more symptoms or measurable markers of a neoplastic disease. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments.

Non-limiting examples of anti-cancer therapies include surgery, radiotherapy, chemotherapy, biological therapy, and combinations thereof.

In particular embodiments, said subject is human. In certain embodiments, the methods or uses as taught herein are useful for indicating treatment with an antineoplastic agent, such as an EGFR inhibitor, a MEK inhibitor, a CAMK inhibitor, and a SRC kinase inhibitor, as a suitable or unsuitable treatment for a neoplastic disease in a subject.

In certain embodiments, if said antineoplastic agent is an antineoplastic agent selected from the group consisting of an EGFR inhibitor, a MEK inhibitor, and a combination thereof,
the determination in a biological sample obtained from said subject of the presence of the genetic or epigenetic alteration leading to reduced or abolished expression or function of FAT1; or
the determination of reduced or abolished FAT1 expression or function in a biological sample obtained from said subject,
   indicates treatment with the antineoplastic agent as an unsuitable treatment (as the subject is likely to have no clinical benefit from the treatment).

In certain embodiments, if said antineoplastic agent is an antineoplastic agent selected from the group consisting of an EGFR inhibitor, a MEK inhibitor, and a combination thereof,
the determination in a biological sample obtained from said subject of the absence of the genetic or epigenetic alteration leading to reduced or abolished expression or function of FAT1; or
the determination of an unchanged or increased FAT1 expression or function in a biological sample obtained from said subject,
   indicates treatment with the antineoplastic agent as a suitable treatment (as the subject is likely to clinically benefit from the treatment).

In particular embodiments, if said antineoplastic agent is an antineoplastic agent selected from the group consisting of a CAMK inhibitor, a SRC kinase inhibitor, and a combination thereof,
the determination in a biological sample obtained from said subject of the presence of the genetic or epigenetic alteration leading to reduced or abolished expression or function of FAT1; or
the determination of reduced or abolished FAT1 expression or function in a biological sample obtained from said subject,
   indicates treatment with the antineoplastic agent as a suitable treatment (as the subject is likely to clinically benefit from the treatment).

In particular embodiments, if said antineoplastic agent is an antineoplastic agent selected from the group consisting of a CAMK inhibitor, a SRC kinase inhibitor, and a combination thereof,
the determination in a biological sample obtained from said subject of the absence of the genetic or epigenetic alteration leading to reduced or abolished expression or function of FAT1; or
the determination of an unchanged or increased FAT1 expression or function in a biological sample obtained from said subject,
   indicates treatment with the antineoplastic agent as an unsuitable treatment (as the subject is likely to have no clinical benefit from the treatment).

The present methods may thus allow predicting treatment with an antineoplastic agent as a suitable treatment. Based on the prediction, the treatment of the neoplastic disease can be initiated, continued or adapted. In certain embodiments, the methods or uses as taught herein are useful for predicting an outcome of treatment with an antineoplastic agent in a subject having a neoplastic disease.

The term “outcome” generally refers to the evaluation undertaken to assess the results or consequences of management and procedures (i.e., the interventions) used in combatting a disease in order to determine the efficacy, effectiveness, safety, practicability, etc., of these interventions, e.g. in individual cases or series. The phrase “predicting outcome” as used herein refers to a process of assessing the consequences of treating an individual afflicted with a neoplastic disease with an antineoplastic agent, i.e. predicting whether said individual is likely to respond or not to the antineoplastic agent. The methods as taught herein provide a prediction of how a patient's neoplastic disease will progress when treated with an antineoplastic agent and whether there is chance of recovery.

In certain embodiments, the methods for determining sensitivity or resistance to treatment with an antineoplastic agent in a subject diagnosed with a neoplastic disease as taught herein is a method for predicting the response of a subject diagnosed with a neoplastic disease to an antineoplastic agent, comprising the steps of:
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject; and
predicting from the presence or absence of a genetic or epigenetic alteration leading to reduced or abolished expression or function of FAT1 or from a reduced or abolished FAT1 expression or function in the biological sample obtained from said subject the response of said subject to said antineoplastic agent;
   wherein said antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor.

In certain embodiments, the methods for determining sensitivity or resistance to treatment with an antineoplastic agent in a subject diagnosed with a neoplastic disease as taught herein is a method of determining sensitivity of neoplastic cell growth to inhibition by an antineoplastic agent, comprising the steps of:
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject; and
determining from the presence or absence of a genetic or epigenetic alteration leading to reduced or abolished expression or function of FAT1 or from a reduced or abolished FAT1 expression or function in the biological sample obtained from said subject the sensitivity of neoplastic cell growth to said antineoplastic agent;
   wherein said antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor.

In certain embodiments, the methods for determining sensitivity or resistance to treatment with an antineoplastic agent in a subject diagnosed with a neoplastic disease as taught herein is a method of determining sensitivity or resistance of a neoplasm in a subject diagnosed with a neoplastic disease to an antineoplastic agent, comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject; and
   determining from the presence or absence of a genetic or epigenetic alteration leading to reduced or abolished expression or function of FAT1 or from a reduced or abolished FAT1 expression or function in the biological sample obtained from said subject the sensitivity or resistance of the neoplasm in said subject diagnosed with a neoplastic disease to said antineoplastic agent,
   wherein said antineoplastic agent is selected from the group consisting of an EGFR inhibitor, a MEK inhibitor, a CAMK inhibitor, and a SRC kinase inhibitor.

In certain embodiments, the methods for determining sensitivity or resistance to treatment with an antineoplastic agent in a subject diagnosed with a neoplastic disease as taught herein is a method for identifying subject with a neoplastic disease as a candidate for treatment with an antineoplastic agent, comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject; and
   identifying the subject as a poor candidate for the treatment with said antineoplastic agent from the presence of a genetic or epigenetic alteration leading to reduced or abolished expression or function of FAT1 or from a reduced or abolished FAT1 expression or function in the biological sample obtained from said subject;
   wherein said antineoplastic agent is an EGFR inhibitor or a MEK inhibitor.

The term “poor candidate” as used herein refers to a subject having a neoplastic disease who will show a minimal (e.g. clinically insignificant) or no response to treatment with said antineoplastic agent.

the methods for determining sensitivity or resistance to treatment with an antineoplastic agent in a subject diagnosed with a neoplastic disease as taught herein is a method for identifying a subject with a neoplastic disease as a candidate for treatment with an antineoplastic agent, comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject; and
   identifying the subject as a good candidate for the treatment with said neoplastic agent from the presence of a genetic or epigenetic alteration leading to reduced or abolished expression or function of FAT1 or from a reduced or abolished FAT1 expression or function in the biological sample obtained from said subject;
   wherein said medicament is a CAMK inhibitor or a SRC kinase inhibitor.

The term “good candidate” as used herein refers to a subject having a neoplastic disease who is sensitive or reactive to treatment with said antineoplastic agent.

The FAT1 status as a marker for determining sensitivity or resistance to treatment with certain antineoplastic agents in a subject diagnosed with a neoplastic disease can also be used as part of a method of treatment, wherein first the sensitivity of the subject to a certain antineoplastic agent is being tested and subsequently, if the subject is considered to be sensitive to said antineoplastic agent, said antineoplastic agent is administered to said subject. If it turns out that the subject is resistant to said antineoplastic treatment, it may be considered to administer to said subject a different anticancer therapy.

Accordingly, a further aspect provides a method of treating a subject diagnosed with a neoplastic disease and/or in need of treatment, comprising

determining the sensitivity or resistance of said subject to treatment with an antineoplastic agent comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject,
wherein said antineoplastic agent is selected from the group consisting of an EGFR inhibitor, a MEK inhibitor, a CAMK inhibitor, and a SRC kinase inhibitor; and
treating said subject with said antineoplastic agent if said subject is determined to be sensitive to said antineoplastic agent.

In other words, the antineoplastic agent selected from the group consisting of an EGFR inhibitor, a MEK inhibitor, a CAMK inhibitor, and a SRC kinase inhibitor may be used in the treatment of a neoplastic disease, comprising:
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject,
wherein said antineoplastic agent is selected from the group consisting of an EGFR inhibitor, a MEK inhibitor, a CAMK inhibitor, and a SRC kinase inhibitor; and
administering said antineoplastic agent to said subject if said subject is determined to be sensitive to said antineoplastic agent or not administering said antineoplastic agent to said subject if said subject is determined to be insensitive to said antineoplastic agent.

A further aspect provides an antineoplastic agent selected from the group consisting of a CAMK inhibitor, a SRC kinase inhibitor, and a combination thereof, for use in the treatment of a neoplastic disease in a subject, wherein said subject has been selected as having a neoplastic disease characterized by
a genetic or epigenetic alteration leading to reduced or abolished expression or function of FAT1, or
reduced or abolished FAT1 expression or function.

In particular embodiments, the methods or the antineoplastic agent for use in the treatment as taught herein comprises administering a therapeutically effective amount of said antineoplastic agent selected from the group consisting of a CAMK inhibitor, a SRC kinase inhibitor, and a combination thereof, to said subject.

A further aspect provides an antineoplastic agent selected from the group consisting of an EGFR inhibitor, a MEK inhibitor, and a combination thereof, for use in the treatment of a neoplastic disease in a subject, wherein said subject has been selected as having a neoplastic disease characterized by
the absence of a genetic or epigenetic alteration leading to reduced or abolished expression or function of FAT1, or
normal or increased FAT1 expression or function.

In particular embodiments, the methods or the antineoplastic agent for use in the treatment as taught herein administering an antineoplastic therapy to said subject different from said antineoplastic agent selected from the group consisting of an EGFR inhibitor, a MEK inhibitor, a CAMK inhibitor, a SRC kinase inhibitor, and a combination thereof, to said subject if said subject is determined to be insensitive to said antineoplastic agent selected from the group consisting of an EGFR inhibitor, a MEK inhibitor, a CAMK inhibitor, a SRC kinase inhibitor, and a combination thereof.

Anti-neoplastic therapies different from said antineoplastic agent selected from the group consisting of an EGFR inhibitor, a MEK inhibitor, a CAMK inhibitor, a SRC kinase inhibitor, and a combination thereof, are known in the art and include surgery, radiotherapy, chemotherapy, biological therapy, and combinations thereof.

As used herein, a phrase such as “a subject in need of treatment” includes subjects that would benefit from treatment of a given condition, particularly a neoplastic disease such as carcinoma. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed neoplastic disease, as well as prophylactic or preventive measures, wherein the aim is to prevent or lessen the chances of incidence of an undesired affliction, such as to prevent occurrence, development and progression of a neoplastic disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

The products for use and methods as taught herein allow to administer a therapeutically effective amount of a antineoplastic agent selected from the group consisting of an EGFR inhibitor, a MEK inhibitor, a CAMK inhibitor, and a SRC kinase inhibitor, in subjects having a neoplastic disease which will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated. Methods are known in the art for determining therapeutically effective doses of an agent as taught herein, such as a modulating agent or antineoplastic agent.

In certain embodiments, said antineoplastic agent selected from the group consisting of an EGFR inhibitor, a MEK inhibitor, a CAMK inhibitor, and a SRC kinase inhibitor is formulated into and administered as pharmaceutical formulations or compositions. Such pharmaceutical formulations or compositions may be comprised in a kit of parts.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

The dosage or amount of the present antineoplastic agents used, optionally in combination with one or more other active compounds to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) as taught herein.

Without limitation, depending on the type and severity of the disease, a typical daily dosage of an antineoplastic as disclosed herein, or combinations of two or more such antineoplastic agents, might range from about 1 μg/kg to 1 g/kg of body weight or more, depending on the factors mentioned above. For instance, a daily dosage of the agent(s) may range from about 1 mg/kg to 1 g/kg of body weight. For repeated administrations over several days or longer, depending on the condition, the treatment is sustained until a desired suppression of disease symptoms occurs.

In certain embodiments, the antineoplastic agent(s) may be administered daily during the treatment. In certain embodiments, the agent(s) may be administered at least once a day during the treatment, for example the antineoplastic agent(s) may be administered at least twice a day during the treatment, for example the antineoplastic agent(s) may be administered at least three times a day during the treatment. In certain embodiments, the antineoplastic agent(s) may be administered continuously during the treatment for instance in an aqueous drinking solution.

In certain embodiments, the antineoplastic agent(s) or pharmaceutical formulation as taught herein may be used alone or in combination with one or more active compounds that are suitable in the treatment of neoplastic diseases (i.e., combination therapy). The latter can be administered before, after, or simultaneously with the administration of the antineoplastic agent(s) or pharmaceutical formulation as taught herein.

The finding of present inventors that the FAT1 status can be used as a marker for determining sensitivity or resistance to treatment with certain antineoplastic agents in a subject diagnosed with a neoplastic disease can also be converted into a kit which allows making that determination of sensitivity or resistance based on the FAT1 status and which can assist a surgeon, researcher, veterinarian, medical doctor or other clinician, in his decision making process on whether to administer a certain antineoplastic agent to a subject diagnosed with a neoplastic disease or not.

Hence, a further aspect provides a kit for determining sensitivity or resistance to treatment with an antineoplastic agent in a subject diagnosed with a neoplastic disease, comprising
means for determining in a biological sample the presence or absence of a genetic or epigenetic alteration leading to reduced or abolished expression or function of FAT1, and/or
means for determining FAT1 expression or function in a biological sample, and a computer readable storage medium having recorded thereon one or more programs for carrying out the method of any one of claims 1 to 6.

The terms “kit of parts” and “kit” as used throughout this specification refer to a product containing components necessary for carrying out the specified methods, packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited set of isolated oligonucleotides as taught herein, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

The kits as taught herein may be used for determining sensitivity to treatment with an antineoplastic agent in a subject having a neoplastic disease, wherein the antineoplastic agent is selected from the group consisting of an EGFR inhibitor, a MEK inhibitor, a CAMK inhibitor, and a SRC kinase inhibitor.

The kits as taught herein may be used for indicating treatment with an antineoplastic agent as a suitable treatment for a neoplastic disease in a subject; or for predicting an outcome of treatment with an antineoplastic agent in a subject having a neoplastic disease, all wherein the antineoplastic agent is selected from the group consisting of an EGFR inhibitor, a MEK inhibitor, a CAMK inhibitor, and a SRC kinase inhibitor. Preferably, the subject is a human subject.

In particular embodiments, the means for determining in a biological sample the presence or absence of a genetic or epigenetic alteration leading to reduced or abolished expression or function of FAT1, or

the means for determining FAT1 expression or function in a biological sample, are one or more agent(s) capable of specifically binding to the FAT1 gene or gene product as described elsewhere herein.

In particular embodiments, the kit may further comprise a reference value corresponding to the genetic or epigenetic alterations in a healthy tissue, preferably wherein a tissue from a healthy subject that is of the same tissue type as the tissue that is afflicted by a pathology in a diseased subject.

In particular embodiments, the kit may further comprise a reference value corresponding to the expression or function of FAT1 in a healthy tissue, preferably wherein a tissue from a healthy subject that is of the same tissue type as the tissue that is afflicted by a pathology in a diseased subject.

The person skilled in the art will understand that the different embodiments of the methods for determining sensitivity or resistance to treatment with an antineoplastic agent in a subject diagnosed with a neoplastic disease also apply to the methods of treating a subject diagnosed with a neoplastic disease as taught herein, the kits as taught herein and the antineoplastic agent for use in the treatment of a neoplastic disease in a subject as taught herein, and vice versa.

The present application also provides aspects and embodiments as set forth in the following Statements:

Statement 1. A method for determining sensitivity or resistance to treatment with an antineoplastic agent in a subject diagnosed with a neoplastic disease, the method comprising
determining in a biological sample obtained from said subject the presence or absence of a genetic or epigenetic alteration leading to reduced or abolished expression or function of FAT1, or
determining whether FAT1 expression or function is reduced or abolished in a biological sample obtained from said subject;
   wherein said antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor.

Statement 2. A method of treating a subject diagnosed with a neoplastic disease, comprising
determining the sensitivity or resistance of said subject to treatment with an antineoplastic agent comprising
   determining in a biological sample obtained from said subject the presence or absence of a genetic or epigenetic alteration leading to reduced or abolished expression or function of FAT1, or
   determining whether FAT1 expression or function is reduced or abolished in a biological sample obtained from said subject,
wherein said antineoplastic agent is selected from the group consisting of an EGFR inhibitor, a MEK inhibitor, a CAMK inhibitor, and a SRC kinase inhibitor; and
treating said subject with said antineoplastic agent if said subject is determined to be sensitive to said antineoplastic agent.

Statement 3. The method according to statement 1 or 2, wherein said antineoplastic agent is an antineoplastic agent selected from the group consisting of an EGFR inhibitor, a MEK inhibitor, and a combination thereof, and wherein
the presence of the genetic or epigenetic alteration leading to reduced or abolished expression or function of FAT1 in said biological sample, or
reduced or abolished expression or function of FAT1 in said biological sample
   indicate that said subject is resistant to treatment with said antineoplastic agent.

Statement 4. The method according to statement 1 or 2, wherein said antineoplastic agent is an antineoplastic agent selected from the group consisting of a CAMK inhibitor, a SRC kinase inhibitor, and a combination thereof, and wherein
the presence of the genetic or epigenetic alteration leading to reduced or abolished expression or function of FAT1 in said biological sample, or
reduced or abolished expression or function of FAT1 in said biological sample
   indicate that said subject is sensitive to treatment with said antineoplastic agent.

Statement 5. The method according to any one of statements 1 to 4, wherein said biological sample comprises neoplastic cells, preferably wherein said biological sample is a tumor biopsy or a liquid biopsy.

Statement 6. The method according to any one of statements 1 to 5, wherein the presence or absence of the genetic or epigenetic alteration or the expression or function of FAT1 is determined using a technique selected from the group consisting of nucleic acid analysis, immunological assay, functional assay, and a combination thereof.

Statement 7. A kit for determining sensitivity or resistance to treatment with an antineoplastic agent, comprising
means for determining in a biological sample the presence or absence of a genetic or epigenetic alteration leading to reduced or abolished expression or function of FAT1, or
means for determining FAT1 expression or function in a biological sample, and
a computer readable storage medium having recorded thereon one or more programs for carrying out the method of any one of statements 1 to 6.

Statement 8. An antineoplastic agent for use in the treatment of a neoplastic disease in a subject, wherein:
the antineoplastic agent is selected from the group consisting of a CAMK inhibitor, a SRC kinase inhibitor, and a combination thereof, and said subject has been selected as having a neoplastic disease characterized by
   a genetic or epigenetic alteration leading to reduced or abolished expression or function of FAT1, or
   reduced or abolished FAT1 expression or function; or
the antineoplastic agent is selected from the group consisting of an EGFR inhibitor, a MEK inhibitor, and a combination thereof, and said subject has been selected as having a neoplastic disease characterized by
   the absence of a genetic or epigenetic alteration leading to reduced or abolished expression or function of FAT1, or
   normal or increased FAT1 expression or function.

Statement 9. The method according to any one of statements 1 to 6 or the antineoplastic agent for use according to statement 8, wherein said neoplastic disease is of epithelial, mesenchymal or melanocyte origin, preferably of epithelial origin.

Statement 10. The method according to statement 9, or the antineoplastic agent for use according to statement 9, wherein the neoplastic disease is a carcinoma originated from epithelial tissue selected from the group consisting of skin, lung, intestine, colon, breast, bladder, head and neck, esophagus, thyroid, kidney, liver, pancreas, bladder, penis, testes, prostate, vagina, cervix, anus, and any combination thereof.

Statement 11. The method according to statement 9 or 10, or the antineoplastic agent for use according to statement 9 or 10, wherein the neoplastic disease is a squamous cell carcinoma (SCC), preferably a SCC of the skin, SCC of the head and neck, SCC of the oesophagus or SCC of the lung.

Statement 12. The method according to any one of statements 1 to 6 or 9 to 11, or the antineoplastic agent for use according to any one of statements 8 to 12, wherein said subject is human.

Statement 13. The method according to any one of statements 1 to 7 or 9 to 12, the kit according to statement 7, or the antineoplastic agent for use according to any one of statements 9 to 12, wherein said CAMK inhibitor is a CAMK2 inhibitor.

Statement 14. The method according to any one of statements 1 to 7 or 9 to 13, the kit according to statement 7, or the antineoplastic agent for use according to any one of statements 9 to 13, wherein said genetic alteration leading to reduced or abolished expression or function of FAT1 function is
one or more FAT1 loss-of-function mutations, or
a copy number variation (CNV) comprising the deletion of the FAT1 gene or a portion thereof.

Statement 15. The method according to statement 14, wherein the one or more FAT1 loss-of-function mutations are selected from the group consisting of a missense mutation, a nonsense mutation, a frameshift mutation, a splicing mutation, and a combination thereof.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1. Materials and Methods for Examples 2 and 3

Compliance with Ethical Regulations.

Mouse colonies were maintained in a certified animal facility in accordance with the European guidelines. All the experiments were approved by the corresponding ethical committee (Commission d'étique et du bien être animal CEBEA, Faculty of Medicine, Universite Libre de Bruxelles). CEBEA follows the European Convention for the Protection of Vertebrate Animals used for Experimental and other Scientific Purposes (ETS No. 123). The mice were checked every day and were euthanized when the tumor reach the end-point size (1 cm in diameter or 1 cm 3 in volume) or if the tumor was ulcerated independently of its size, if the mouse lost >20% of the initial weight or any other sign of distress (based on the general health status and spontaneous activity). None of the experiments performed in this study surpassed the size limit of the tumors. All the experiments strictly complied with the protocols approved by ethical committee. Patient Derived Xenografts (PDX) used in this study to analyse hybid EMT state in human Fat1 mutated cancers are part of PDX project, that has been approved by the Ethical Committee of the Hospital Erasme (Universite Libre de Bruxelles) and by Ethical Committees in all hospitals involved in the patient recruitment. Informed consent was obtained from all patients.

### Mouse Strains.

K14Cre (Vasioukhin, V., et al. Hyperproliferation and defects in epithelial polarity upon conditional ablation of alpha-catenin in skin. Cell, 2001, vol. 104, 605-617), K14CreER (Vasioukhin, V., et al. The magical touch: genome targeting in epidermal stem cells induced by tamoxifen application to mouse skin. Proc Natl Acad Sci USA, 1999, vol. 96, 8551-8556), Fat1fl/fl (Caruso, N. et al. Deregulation of the protocadherin gene FAT1 alters muscle shapes: implications for the pathogenesis of facioscapulohumeral dystrophy. PLoS Genet, 2013, vol. 9, e1003550), Rosa26-YFP (Srinivas, S. et al. Cre reporter strains produced by targeted insertion of EYFP and ECFP into the ROSA26 locus. BMC Dev Biol, 2001, vol. 1, 4), Lgr5CreER (Barker, N. et al. Identification of stem cells in small intestine and colon by marker gene Lgr5. Nature, 2007, vol. 449, 1003-1007), KRasLSL-G12D (Barker, N. et al. Identification of stem cells in small intestine and colon by marker gene Lgr5. Nature, 2007, vol. 449, 1003-1007) and p53fl/fl (Jonkers, J. et al. Synergistic tumor suppressor activity of BRCA2 and p53 in a conditional mouse model for breast cancer. Nat Genet, 2001 vol. 29, 418-425) mice have been previously described or imported from the NCI mouse repository and the Jackson Laboratories. NOD/SCID/112Rγ null mice were purchased from Charles River. All mice used in this study were composed of males and females with mixed genetic background. No randomization and no blinding were performed in this study.

KRas^{LSL-G12D}p53^{fl/fl} Driven Skin Tumors.

Tamoxifen was diluted at 25 mg/ml in sunflower seed oil (Sigma). Four daily intraperitoneal injection (IP) doses of 2.5 mg of Tamoxifen were administered at P28 as previously described (Lapouge, G. et al., Skin squamous cell carcinoma propagating cells increase with tumour progression and invasiveness, EMBO, 2012, vol. 31: 4563-4575) to Lgr5CreER/Kras^{LSL-G12D}/p53^{fl/fl}/Fat1^{fl/fl}/Rosa26^{YFP/+} and 1 IP injection dose of 2.5 mg of Tamoxifen was administrated to K14CreER/Kras^{LSL-G12D}/p53^{fl/fl}/Fat1^{fl/fl}/Rosa26^{YFP/+}. Tumor appearance and size were detected by daily observation and palpation. Mice were euthanized when tumor size reached 2 cm (Gendoo, D. M. et al. Genefu: an R/Bioconductor package for computation of gene expression-based signatures in breast cancer. Bioinformatics, 2016, vol. 32, 1097-1099) or when mice presented signs of distress. Skin tumors were measured using a precision calliper allowing to discriminate 0.1 mm modifications in size. tumor volumes were measured the first day of appearance of the tumor and then, every week until the death of the animal with the formula V=π×[d²×D]/6, where d is the minor tumor axis and D is the major tumor axis.

KRas^{LSL-G12D}p53^{fl/fl} Driven Lung Tumors.

6-12 week old Kras^{LSL-G12D}/p53^{fl/fl}/Rosa26^{YFP/+} and Kras^{LSL-G12D}/p53^{fl/fl}/Fat1^{fl/fl}/Rosa26^{YFP/+} mice were anaesthetized and received a single 50 μl bolus of 2.5*10⁷ PFU Ad5CMVCre virus (University of Iowa, Viral Vecor Core Facility) by intratracheal instillation, as previously described (DuPage, M., et al. Conditional mouse lung cancer models using adenoviral or lentiviral delivery of Cre recombinase. Nat Protoc, 2009, vol. 4, 1064-1072). The mice were followed by daily observation and weight measure. Mice were sacrificed if >5% weight loss in 1-2 days, >20% weight loss from the beginning of the experiment, if the consumption of water or food decreased significantly or whether any abnormal behavior was observed (breathing difficulties, passivity or edema).

### Collection of Human Samples

Tumor samples and corresponding blood samples were collected directly from the surgery room from patients diagnosed with squamous cell carcinomas from different organs of origin. The tumor was collected in RMPI culture medium supplemented with 10% FBS, and 1% penicillin/streptomycin and were stored or shipped at 4 degrees. Blood sample was collected in EDTA tube and stored or shipped at 4 degrees. Tumor samples were processed directly or if the size of the sample was small it was transplanted into immunodeficient mice to create Patient Derived Xenografts (PDX).

### Sample Size, Data Exclusions, Randomization and Blinding.

The sample size was chosen based on previous experience in the laboratory, for each experiment to yield high power to detect specific effects. No statistical methods were used to predetermine sample size. For in vivo studies on primary mouse models, animals were chosen based on correct genotypes: requiring 2 or 3 correct alleles for DMBA/TPA tumors (K14Cre/RYFP, K14Cre/Fat1/RYFP, K14CreER/Fat1/RYFP) or 5 correct alleles for genetic tumors (Lgr5CreER/Kras/p53/Fat1/RYFP or K14CreER/Kras/p53/Fat1/RYFP). All animals were initiated the treatment with DMBA/TPA at 6-8 weeks of age or were induced with Tamoxifen at 28-35 days after birth. Sex-specific differences were minimized by including similar number of male and female animals if possible. Investigators were blinded to mouse genotypes during the analysis, imaging and quantifications (histology analysis, FACS, cytospin quantifications, metastasis quantification.

### Definition and FACS Isolation of Tumor Cell Subpopulations

Skin tumors from Lgr5CreER/Kras^{G12D}/p53^{fl/fl}/Fat1^{cKO}/RYFP and K14CreER/Kras^{G12D}/p53^{fl/fl}/Fat1^{cKO}/RYFP mice were dissected, minced and digested in collagenase type I (Sigma) at 3.5 mg/ml during 1 hour at 37° C. on a rocking plate protected from light. Collagenase activity was blocked by the addition of EDTA (5 mM) and then the cells were rinsed in PBS supplemented with 10% FBS and the cell suspensions were filtered through a 70 um cell strainers (BD Bioscience). Brilliant violet stain buffer (BD Bioscience) was added (50 μl/sample) and the cells were incubated with PE-conjugated anti-CD51 (rat clone RMV-7, Biolegend Cat #104106, dilution 1:50), BV421-conjugated anti-CD61 (Armenian hamster, clone 2C9.G2, BD Bioscience Cat #553345, dilution 1:50), biotin-conjugated anti-CD106 (rat, clone 429 (MVCAM.A), BD Bioscience Cat #553331, dilution 1:50), BV711-conjugated anti-Epcam (rat clone G8.8, BD Bioscience Cat #563134, dilution 1:100), PerCPCy5.5 conjugated anti-CD45 (rat, clone 30-F11, BD Bioscience Cat #550994, dilution 1:100) and PerCPCy5.5 conjugated anti-CD31 (rat, clone MEC 13.3, BD Bioscience Cat #562861, dilution 1:100) during 30 min at 4° C. protected from light. Cells were washed with PBS supplemented with 2% FBS and incubated with streptavidin-BV786 (BD Bioscience Cat #563858, dilution 1:400) during 30 min at 4° C. protected from light. Living single tumor cells were selected by forward and side scatter, doublets discrimination and by 7AAD dye exclusion. Tumor cells were selected based on YFP expression and the exclusion of CD45 and CD31 (Lin−). Different tumor cell subpopulations were defined in Epcam-tumor cells by combination of CD61, CD51 and CD106 markers.

CD44 expression in FAT1 WT and FAT1 KO human SCC cells was performed after trypsinization of cells and incubation with APC conjugated anti-CD44 (Clone IM7, BioLegend Cat #103011, dilution 1:50) antibody. Living tumor cells were selected by forward and side scatter, doublets discriminating and DAPI dye exclusion.

Fluorescence-activated cell sorting and analysis were performed using FACSAria and LSRFortessa, using FACSDiva software (BD Bioscience). Sorted cells were collected in culture medium (for in vivo transplantation experiments), lysis buffer (for RNA extraction) or in PBS supplemented with 3% FBS (for ATAC).

### Tumor Transplantation Assays

The different FACS isolated tumor cell subpopulations (YFP+/Epcam+, YFP+/Epcam+/Fat1^{fl/fl}, YFP+/Epcam−/Fat1^{fl/fl} from K14Cre/RYFP and K14Cre/Fat1^{fl/fl}/RYFP) were collected in 4° C. medium. Cells at different dilutions (10,000, 1000, 100 and 10 cells) were resuspended in 50 ul of Matrigel (E1270, 970 mg/ml; Sigma) and injected subcutaneously to NOD/SCID/Il2Rγ null mice. Secondary tumors were detected by palpation every week and their size monitored until tumor reached 1 cm³ or when mice presented signs of distress. The mice were sacrificed and the number of secondary tumors was quantified. The tumor propagating cell frequency was computed using the extreme limiting dilution analysis (ELDA) online software as previously described (http://bioinf.wehi.edu.au/software/elda/) (Hu, Y. & Smyth, G. K. ELDA: extreme limiting dilution analysis for comparing depleted and enriched populations in stem cell and other assays. J Immunol Methods, 2009, vol. 347, 70-78).

### Metastasis Assay

The different FACS isolated tumor cell subpopulations were collected in 4° C. medium. Cells were resuspended in PBS in 50 ul PBS and injected to the vein tail of the NOD/SCID/Il2Rγ null mice (20,000 cells per injection). Mice were sacrificed at 40 days and the lungs were analyzed to detect the presence of metastasis. The number of metastasis was quantified on 10 cryosections per lungs (separated by 100 um) based on YFP and presented as number of metastasis per lungs. The metastases were characterized by K14 and Vimentin expression by immunofluorescence.

### Primary Cell Culture

Freshly isolated FACS-isolated YFP+ Epcam− tumor cells were plated in 6-well plates. Cells were cultured in EMEM medium supplemented with 15% FBS, 0.4 ug/ml hydrocortisone, 10 ng/ml EGF, 2×10-9M T3, 1% penicillin/streptomycin and 2 mM L-glutamin. The cells were incubated at 37 degrees with 20% 02 and 5% CO2.

### Cell Culture Human Cell Line

A388 cells (human skin SCC) cells were maintained in DMEM medium supplemented with 10% FBS, 100 U/ml Pen/Strep and 2 mM L-glutamine. 293T cells were maintained in DMEM (Gibco) supplemented with 10% FBS, 2 mM Glutamine, 100 U/ml Pen/Strep solution, MEM Non-Essential Amino Acids Solution and Pyruvate. All cell lines were acquired from ATCC and cultured at 37 degrees with 20% 02 and 5% CO2.

### Western Blot

The cells were lysed in cell lysis buffer (Cell Signaling, catalog #9803) with phosphatase inhibitor cocktail (Cell Signaling, catalog 5870) and 1 mM PMSF (Sigma, Cat #P7626) for 5 minutes on ice, sonicated 5 times 10 seconds and then centrifuged for 10 min at 14.000 rpm at 4° C. A range of 5 to 30 micrograms of cell lysate was loaded in NuPage 10% Bis-Tris gel (Invitrogen, Cat #NP0315BOX) and separated by electrophoresis. Proteins were transferred on nitrocellulose membranes (Thermo Scientific, Cat #88018). The membranes were incubated overnight with anti-phospho-CAMK2 (Rabbit, 1/133, Cell Signaling, clone D21E4, cat #12716) or anti-phospho-SCR Tyr416 (Rabbit, 1:3000, Cell Signaling, clone D49G4, Cat #6943) or anti-H3K27Me3 Lys27 (Rabbit, 1:3000, Millipore, Cat #17-622) or anti-phospho-MEK1/MEK2 Ser218, SER222, Ser226 (Rabbit, 1:1000, Invitrogen, Cat #44-454G) or anti-phospho-EGFR Y1197 (Rat, 1:500, R&D, MAB8058), anti-CAMK2 (pan) (Rabbit, 1/125, Cell Signaling, clone D11A10, cat #4436) or anti-SRC (Rabbit, 1:1000, Cell Signaling, clone32G6, Cat #2123) or anti-H3 (Rabbit, 1:6000, Abcam, Cat #ab1791) or anti-MEK1/MEK2 (Rabbit, 1:1000, Invitrogen, Cat #PA5-31917) or anti-EGFR (Rabbit, 1:1000, Cell Signaling, clone D38B1, Cat #4267), anti-YES (Rabbit, 1:1000, Cell Signaling, clone D9P3E, Cat #65890) and anti-β-actin (1:2000, Abcam, Cat #ab8227). Anti-rabbit or anti-rat immunoglobulin G (IgG) conjugated with horseradish peroxidase (HRP) (1:3000 or 1:5000; Healthcare) was used as the secondary antibody.

Treatment with Pharmacological Inhibitors

For treatments with pharmacological inhibitors the media was supplemented with different inhibitors as described below, incubated at 37 degrees with 20% O2 and 5% CO2 and collected for analysis.

For CAMK2 inhibition present inventors used CAMK2 inhibitor KN93 (Selleckchem Cat #57423, dissolved in DMSO) at 10 μM and present inventors incubated cells during 96 h refreshing the medium once after 48 h. After 96 h the cells were collected for WB as described above or for FACS analysis. For immunostaining present inventors followed the same treatment but the cells were plated previously on coverslips covered with gelatin.

For YES/SRC inhibition present inventors used Saracatinib (Selleckchem Cat #S1006, dissolved in DMSO) at 500 nM or Dasatinib (Sellechchem Cat #S1021, dissolved in DMSO) at 100 nM for 72 h refreshing the medium once after 48 h. After 72 h the cells were collected for WB or immunostaining as described above.

For EZH2 inhibition present inventors used GSK343 (Selleckchem Cat #S7164, dissolved in DMSO) at 500 nM for 7 days, refreshing the medium every 48 h. After 7 days the cells were collected for WB or immunostaining as described above.

### Drug Sensitivity Assay

For the drug sensitivity assay, cells have been seeded at a density of 4000 cells/well in a 96 well plate. 24 hours after seeding, the cells were treated with serial dilution of all the inhibitors (Afatinib—Selleckchem Cat #S1011, Trametinib—Selleckchem Cat #S2673, Saracatinib—Selleckchem Cat #S1006, Dasatinib—Sellechchem Cat #S1021 or KN93—Selleckchem Cat #S7423), vehicle control (DMSO) or positive control (puromycin) in 5% FBS medium. 48 hours after the start of treatment, cells have been harvested by trypsinization, and quantified by counting the number of living cells by FACS. Living cells were selected by forward and side scatter and by Hoechst dye exclusion. Each data point is the result of at least three independent replicates; each of the replicates is evaluated using a technical duplicate. Drug response curves and IC50 values have been calculated using the Prism8 software.

### Soft Substrates Fabrication and YAP1 Quantification

For the fabrication of soft substrates, present inventors transferred ECM proteins onto polyacrylamide gels as in (Vignaud, T., Ennomani, H. & Thery, M. Polyacrylamide hydrogel micropatterning. Methods Cell Biol, 2014, vol. 120, 93-116). Glass coverslips were first incubated with 10 μg/ml of fibronectin (F1141, Sigma) and 10 μg/ml of type I collagen extracted from rat tails, in 100 mM sodium bicarbonate (144-55-8, Sigma) solution at pH 8.3 for 30 minutes.

Polyacrylamide gels were prepared by polymerizing a mix of acrylamide (01697, Fluka Analytical, USA) and bis-acrylamide (66675, Fluka Analytical, USA) in a respective ratio of 10%/0.03% for the 3 kPa gels, and 8%/0.48% for the 40 kPa gels. The polyacrylamide mix was de-gassed for 20 minutes in a vacuum chamber and 165 μl was mixed with 1 μl of tetramethylethylenediamine (TEMED) (T9281, Sigma, USA) and 1 μl of 10% ammonium persulfate (APS) (A3678, Sigma, USA). The polymerization was achieved on the coated coverslips for 30 minutes under silanized coverslips. The samples were then immersed in sterile PBS (14190, Gibco) and the silanized coverslips were removed with tweezers and stored in sterile PBS at 4° C. Samples were washed in sterile PBS before cell seeding.

For the quantifications of YAP, present inventors manually defined the cytoplasm and the nucleus regions in ImageJ, and the fluorescence intensities were measured. The densities were then calculated regarding the area of each region and the nucleus/cytoplasm ratios were finally calculated.

### Generation of CRISPR/Cas9 KO Cell Lines and Overexpression (OE) Cell Lines.

For the generation of stable OE cell lines, VSV-G pseudotyped lentivirus was produced by Lipofectamine 2000 transfection (Invitrogen) of HEK293T cells with the shRNA-carrying vector or the corresponding control, and the helper plasmids pMD2-VSVg and pPAX2 (Addgene plasmid 12259 and 12260, respectively). 293T cells were maintained in DMEM (Gibco) supplemented with 10% FBS, 2 mM Glutamine, 100 U/ml Pen/Strep solution, MEM Non-Essential Amino Acids Solution and Pyruvate. 48 h after transfection, present inventors collected the viral supernatant and filtered it through polyvinylidene difluoride filters. For infection, Epcam cells isolated from primary tumors by FACS and grown in culture were plated in a single well of a 6-well plate at 60-70% confluence and incubated with the viral supernatant added with 30 μg/mL Polybrene Infection/transfection reagent (Millipore, TR-1003-G). Present inventors changed medium after 12-16 h. Forty-eight hours after the infection, present inventors started the selection in puromycin containing medium (20 μg/mL puromycin, Invivogen). After one week of puromycin selection, cells were tested for CDH1 expression by immunostaining and FACS.

Present inventors generated the FAT1 KO cell lines in A388 cells by designing four guide RNAs targeting Exon 2. The most efficient guide RNAs were predicted using the MIT CRISPR design tool (Zhang Lab, MIT 2015, now discontinued). The gRNA sequences were cloned as double stranded oligonucleotides in the pSpCas9n (BB)-2A-GFP (PX461) plasmid (Addgene Plasmid #48140), expressing the Cas9 D10A mutant and EGFP simultaneously. Present inventors designed the guides in order to generate a deletion of about 400 bp when the DNA sequence included between the double strand break generated by each couple of guides gets recombined upon DNA repair. The sequence of the guides is available in Table 1. For the generation of CRISPR clones, present inventors seeded A388 cells at 60-70% density, and transfected them the day after seeding with an equimolar amount of each guide using the standard Lipofectamine 2000 transfection protocol (Invitrogen). 48 h after transfection, present inventors isolated the transfected cells based on GFP fluorescence and plated them as single cells in 96 well plates. As soon as single cell clones were visible, present inventors amplified them, extracted genomic DNA and probed them by PCR with a combination of primers detecting the presence or absence of the deletion. Present inventors further validated the deleted clones by sequencing the deletion site using PCR primers flanking the position of the guide RNAs, ensuring that the occurring deletion generated a frame shift in the coding sequence. The sequence of the primers is available in Table 2.

The generation of Fat1/Sox2 double KO, Fat1/Yap1 double KO Fat1/Yap1/Taz triple KO, FAT1/CD44 double KO and of CDH1 KO cell lines has been performed by using single gRNA mediated deletion upon WT Cas9 expression in pre-existing FAT1 KO or WT cell lines. Briefly, present inventors used lentiviral-mediated transduction of the lentiCRISPRv2 plasmid (Addgene Plasmid #52961) or of the lentiCRISPRv2-mCherry plasmid (Addgene Plasmid #99154), followed by puromycin selection or mCherry based sorting of the transduced cells, respectively. In all cases, present inventors tested 4 different gRNA for efficiency, using different approaches to test the knockout efficiency (Immunofluorescence, western blot or FACS), depending on antibody availability. Present inventors designed the gRNAs based on the CHOPCHOP web tool (http://chopchop.cbu.uib.no) or the BROAD institute designed guides available in the Brunello library (for human genes) or the Brie library (for mouse genes). All sequences are available in the Table 1.

TABLE 1
Sequence of the guides used for
FATI CRISPR deletion

| | SEQ ID | |
|---|---|---|
| Name | NO | sequence |
| hFat1_L1_exon2 + | 8 | CACCGAGTGGCTGCCAGTCACAAGC |
| hFat1_L1_exon2 − | 9 | AAACGCTTGTGACTGGCAGCCACTC |
| hFat1_L2_exon2 + | 10 | CACCGTATGTTGATATATAATGGTG |
| hFat1_L2_exon2 − | 11 | AAACCACCATTATATATCAACATAC |
| hFat1_R1_exon2 + | 12 | CACCGACTGGGGCTTGCCGTACCGC |
| hFat1_R1_exon2 − | 13 | AAACGCGGTACGGCAAGCCCCAGTC |
| hFat1_R2_exon2 + | 14 | CACCGATCCTCAGAGTATAAACCCG |
| hFat1_R2_exon2 − | 15 | AAACCGGGTTTATACTCTGAGGATC |
| hCD44 del sg1For | 16 | CACCGAAGACTCCCATTCGACAACA |
| hCD44 del sg1Rev | 17 | AAACTGTTGTCGAATGGGAGTCTTC |
| hCD44 del sg2For | 18 | CACCGCATCACGGTTAACAATAGCT |
| hCD44 del sg2Rev | 19 | AAACAGCTATTGTTAACCGTGATGC |
| hCD44 del sg3For | 20 | CACCGTCGCTACAGCATCTCTCGGA |
| hCD44 del sg3Rev | 21 | AAACTCCGAGAGATGCTGTAGCGAC |
| hCD44 del sg4For | 22 | CACCGTGCTACTTCAGACAACCACA |
| hCD44 del sg4Rev | 23 | AAACTGTGGTTGTCTGAAGTAGCAC |
| hYAP1 del sg1For | 24 | CACCGGATGAACCTTTACCAAAACG |
| hYAP1 del sg1Rev | 25 | AAACCGTTTTGGTAAAGGTTCATCC |
| hYAP1 del sg2For | 26 | CACCGGTGCACGATCTGATGCCOGG |
| hYAP1 del sg2Rev | 27 | AAACCCGGGCATCAGATOGTGCACC |
| hYAP1 del sg3For | 28 | CACCGTCGAACATGCTGTGGAGTCA |
| hYAP1 del sg3Rev | 29 | AAACTGACTCCACAGCATGTTCGAC |
| hYAP1 del sg4For | 30 | CACCGTGCCCCAGACCGTGCCCATG |
| hYAP1 del sg4Rev | 31 | AAACCATGGGCACGGTCTGGGGCAC |
| hSOX2 del sg1For | 32 | CACCGATTATAAATACCGGCCCCGG |
| hSOX2 del sg1Rev | 33 | AAACCCGGGGCCGGTATTTATAATC |
| hSOX2 del sg2For | 34 | CACCGCGTTCATCGACGAGGCTAAG |
| hSOX2 del sg2Rev | 35 | AAACCTTAGCCTCGTCGATGAACGC |
| hSOX2 del sg3For | 36 | CACCGGACAGTTACGCGCACATGAA |
| hSOX2 del sg3Rev | 37 | AAACTTCATGTGCGCGTAACTGTCC |
| hSOX2 del sg4For | 38 | CACCGGCAGGGCGCTCACGTOGTAG |
| hSOX2 del sg4Rev | 39 | AAACCTACGACGTGAGCGCCCTGCC |
| hCDH1 del sg1For | 40 | CACCGAAGTCACGCTGAATACAGTG |
| hCDH1 del sg1Rev | 41 | AAACCACTGTATTCAGCGTGACTTC |
| hCDH1 del sg2For | 42 | CACCGCTGGAGATTAATCCGGACAC |
| hCDH1 del sg2Rev | 43 | AAACGTGTCCGGATTAATCTCCAGC |
| hCDH1 del sg3For | 44 | CACCGGCAATGCGTTCTCTATCCAG |
| hCDH1 del sg3Rev | 45 | AAACCTGGATAGAGAACGCATTGCC |
| hCDH1 del sg4For | 46 | CACCGTGAACCACCAGGGTATACGT |
| hCDH1 del sg4Rev | 47 | AAACACGTATACCCTGGTGGTTCAC |
| mYap1 sg1Rev | 48 | AAACCGGGGACTCGGAGACCGACTC |
| mYap1 sg2For | 49 | CACCGTGCCGTCATGAACCCCAAGA |
| mYap1 sg2Rev | 50 | AAACTCTTGGGGTTCATGACGGCAC |
| mYap1 sg3For | 51 | CACCGGGCGGCTTGAAGAAGGAGTC |
| mYap1 sg3Rev | 52 | AAACGACTCCTTCTTCAAGCCGCCC |
| mYapl sg4For | 53 | CACCGCGACAGGTAAGGGTATCCOG |
| mYap1 sg4Rev | 54 | AAACCGGGATACCCTTACCTGTCGC |
| mSox2 sg1For | 55 | CACCGAGGAGGCAACGCCACGGOGG |
| mSox2 sg1Rev | 56 | AAACCCGCCGTGGCGTTGCCTCCTC |
| mSox2 sg2For | 57 | CACCGTGGGCCTCTTGACGCGGTCC |
| mSox2 sg2Rev | 58 | AAACGGACCGCGTCAAGAGGCCCAC |
| mSox2 sg3For | 59 | CACCGGGTGGGCGAGCCGTTCATGT |
| mSox2 sg3Rev | 60 | AAACACATGAACGGCTCGOCCACCC |
| mSox2 sg4For | 61 | CACCGGGGCACCCCCGGTATGGCGC |
| mSox2 sg4Rev | 62 | AAACGCGCCATACCGGGGGTGCCCC |
| mCdh1 sg1For | 63 | CACCGGGAGAACGAGGAACCCTTTG |
| mCdh1 sg1Rev | 64 | AAACCAAAGGGTTCCTCGTTCTCCC |
| mCdh1 sg2For | 65 | CACCGGTCCACAGTGACAGTGGCTG |
| mCdh1 sg2Rev | 66 | AAACCAGCCACTGTCACTGTGGACC |
| mCdh1 sg3For | 67 | CACCGGTCGAAGTGCCCGAAGACTT |
| mCdh1 sg3Rev | 68 | AAACAAGTCTTCGGGCACTTCGACC |
| mCdh1 sg4For | 69 | CACCGGAACGTGTCCGGCTCTCGAG |
| mCdh1 sg4Rev | 70 | AAACCTCGAGAGCCGGACACGTTCC |
| mTAZ del sg1For | 71 | CACCGACATAGAGAAAATCACCACA |
| mTAZ del sg1Rev | 72 | AAACTGTGGTGATTTTCTCTATGTC |
| mTAZ del sg2For | 73 | CACCGGCAAGTCATCCACGTCACGC |
| mTAZ del sg2Rev | 74 | AAACGCGTGACGTGGATGACTTGCC |
| mTAZ del sg3For | 75 | CACCGTGGGTTGGTTCTGAGTCGGG |
| mTAZ4 del sg3Rev | 76 | AAACCCCGACTCAGAACCAACCCAC |
| mTAZ del sg4For | 77 | CACCGGAGGATTAGGATGCGTCAAG |
| mTAZ del sg4Rev | 78 | AAACCTTGACGCATCCTAATCCTCC |

EXTENDED DATA TABLE 2
Sequence of the primers used for
screening the hFATI CRISPR clones

| | SEQ | |
|---|---|---|
| | ID | |
| Name | NO | sequence |
| Fat1_exon2_external_Forward | 79 | GCGGTATGTGAGC |
| | | ATTGACAG |
| Fat1_exon2_internal_Reverse | 80 | CCCAACTCGGGGG |
| | | TATTGTC |
| Fat1_exon2_external_Reverse | 81 | GGGTGAGAACGGG |
| | | TACGTG |

### RNA Extraction and Real-Time PCR

RNA extraction from FACS isolated cells was performed using RNeasy micro kit (QIAGEN) according to the manufacturer's recommendations. For real-time PCR, after nanodrop quantification, the first strand cDNA was synthesized using Superscript II (Invitrogen) and random hexamers (Roche) in 50111 final volume. Control of genomic contaminations was measured for each sample by performing the same procedure with or without reverse transcriptase. Quantitative PCR assays were performed using 1 ng of cDNA as template, SYBRGreen mix (Applied Bioscience) on Light Cycler 96 (Roche) real-time PCR system. HPRT housekeeping gene was used for normalization. Primers were designed using Pubmed tool (https://tumor.ncbi.nlm.nih.gov/tools/primer-blast/) or Roche Universal Probe Library Assay Design center (https://life science.roche.com/webapp/wcs/stores/servlet/CategoryDisplay?tab=Assay+Design+Center&identifier=Universal+Probe+Library&langId=−1).

### RNA-Sequencing

Prior to sequencing the quality of RNA was evaluated by Bioanalyzer 2100 (Agilent). Indexed cDNA libraries were obtained using the Ovation Solo RNA-seq Systems (NuGen) following manufacturer's recommendations. The multiplexed libraries (11 pM/18 pM) were loaded on flow cells and sequences were produced using a HiSeq PE Cluster Kit v4 and TruSeq SBS Kit v3-HS (250 cycles) on a HiSeq 1500 (Illumina). Reads were mapped against the mouse reference genome (Grcm38/mm10) using STAR software (Dobin, A. et al. STAR: ultrafast universal RNA-seq aligner. Bioinformatics, 2013, vol. 29, to generate read alignments for each sample. Annotations Grcm38.87 was obtained from ftp.Ensembl.org. After transcripts assembling, gene level counts were obtained using HTseq and normalized to 20 millions of aligned reads. Average expression for each gene for the different tumor cell populations was computed based on at least 2 biological replicates and fold changes were calculated between the subpopulations. Genes having a fold change of expression greater or equal than 1.5 or 2 are considered as up-regulated and those having a fold change of expression lower or equal to 0.5 or 0.66 are considered down-regulated.

### Exome-Sequencing Human Tumors and Matched Blood Samples

Whole-genome DNA libraries were created with the KAPA Hyper prep kit (Roche) V2 according to the manufacturer's instructions. The resulting whole-genome libraries were then sequenced on Illumina NovaSeq 6000 generating 75-bp single-end reads, average coverage of 0.1×.

### Exome-Sequencing Analysis

Before starting the alignment and downstream analysis, quality check was done by FastQC (https://tumor.bioinformatics.babraham.ac.uk/projects/fastqc/). Adaptors sequences were removed with TrimmomaticPE using options “ILLUMINACLIP:adaptor.file:2:30:10 LEADING:3 TRAILING:3 SLIDINGWINDOW:4:15 MINLEN:36”. The mouse-contaminated sequence reads which are mapped to mouse reference genome mm10 were filtered out with bbsplit.sh which belongs to BBMap (https://github.com/BioInfoTools/BBMap), the reads which are not mapped to human reference genome hg38 also filtered out. On this step, the options “ambiguous=best ambiguous2=toss maxindel=900000 qtrim=f untrim=f minratio=0.65” were used.

Paired-end reads were then aligned with Burrows-Wheeler Alignment Tool (Li, H. & Durbin, R. Fast and accurate short read alignment with Burrows-Wheeler transform. Bioinformatics, 2009, vol. 25, 1754-1760), under aln mode, with human reference genome hg38. With Picard tools (http://broadinstitute.githubio/picard/), the reads were sorted and duplicated reads were removed. The local realignment process were done with The Genome Analysis Toolkit (GATK)(McKenna, A. et al. The Genome Analysis Toolkit: a MapReduce framework for analyzing next-generation DNA sequencing data. Genome Res 2010, vol. 20, 1297-1303) and all mate-pair information between each read and its mate pair was verified by picard tools.

Coverage of regions was calculated by Picard CalculateHsMetrics and the regions with a mean coverage at least 10 were used for mutation calling. The base quality scores of selected regions were recalibrated with GATK. Variants for tumor and normal were called with GATK, Haplotype Caller function.

To get the somatic mutation, the variants from normal were filtered out from those from tumor by bedtools (Version 2.27.0) (Quinlan, A. R. & Hall, I. M. BEDTools: a flexible suite of utilities for comparing genomic features. Bioinformatics, 2010, vol. 26, 841-842) intersect. The somatic variants were annotated by ANNOVAR (v2013Jun21) (Wang, K., Li, M. & Hakonarson, H. ANNOVAR: functional annotation of genetic variants from high-throughput sequencing data. Nucleic Acids Res, 2010, vol. 38, e164), the program called annotate_variation.pl and table_annovar.pl, using corresponding reference genome. Synonymous substitution was excluded in this study.

### Copy Number Variation (CNV) Analysis

Before starting the alignment and downstream analysis, quality check was done by FastQC (https://tumor.bioinformatics.babraham.ac.uk/projects/fastqc/). Adaptors sequences were removed with TrimmomaticPE using options “ILLUMINACLIP: adaptor. file:2:30:10 LEADING:3 TRAILING:3 SLIDINGWINDOW:4:15 MINLEN:36” (Bolger, A. M., Lohse, M. & Usadel, B. Trimmomatic: a flexible trimmer for Illumina sequence data. Bioinformatics, 2014, vol. 30, 2114-2120). The mouse-contaminated sequence reads were filtered out with bbsplit.sh which belongs to BBMap (https://github.com/BiolnfoTools/BBMap), the sequence which is not mapped to hg19 is also filtered out. On this step, the options “ambiguous=best ambiguous2=toss maxindel=900000 qtrim=f untrim=f minratio=0.65” were used.

The filtered reads were mapped to human reference genome hg38, with Burrows-Wheeler Alignment Tool, under mem mode. The read group information of aligned reads was substituted by picard AddOrReplaceReadGroups and sorted with picard SortSam. The duplicated reads were marked and excluded with picard MarkDuplicates algorithm.

The chromosomal aberration is quantified by QDNAseq (version 1.22.0), which belongs to Bioconductor (https://www.bioconductor.org) (version 3.10) package, with R version 3.6 (https://www.R-project.org/) (Robinson, M. D., McCarthy, D. J. & Smyth, G. K. edgeR: a Bioconductor package for differential expression analysis of digital gene expression data. Bioinformatics, 2010, vol. 26, 139-140; Scheinin, I. et al. DNA copy number analysis of fresh and formalin-fixed specimens by shallow whole-genome sequencing with identification and exclusion of problematic regions in the genome assembly. Genome Res, 2014, vol. 24, 2022-2032). The size of non-overlapping bin was 30 kb, and copy number was quantified under the human reference genome hg19. Quantified copy number were adjusted with simultaneous two-dimensional loess correction based on mappability and GC contents. Also, the genomic regions which are spurious were filtered out.

The heatmap was generated on the bins including interested regions. For getting heatmap, Heatmapper was used (Babicki, S. et al. Heatmapper: web-enabled heat mapping for all. Nucleic Acids Res, 2016, vol. 44, W147-153), the application called expression-based heat maps. The heatmap was generated without scale of dataset.

### ATAC-Sequencing

For ATAC sequencing 100000 sorted cells from different tumor subpopulations were collected in 1 mL of PBS supplemented with 3% FBS at 4° C. Cells were centrifuged and cell pallets were resuspended in 100 ul of lysis buffer (TrisHCl 10 mM, NaCl 10 mM, MgCl2 3 mM, Igepal 0.1%) and centrifuged at 500 g for 25 minutes at 4° C. Supernatant was carefully discarded and nuclei were resuspended in 50 ul of reaction buffer (Tn5 transposase 2.5 ul, TD buffer 22.5 ul, from Nextera DNA sample preparation kit, Illumina, and 25 ul H2O). The reaction was performed at 37° C. for 30 min and was stopped by addition of 5 ul of clean up buffer (NaCl 900 mM, EDTA 300 mM). DNA was purified using the MiniElute purification kit (QIAGEN) following manufacturer protocol. DNA libraries were PCR amplified (Nextera DNA Sample Preparation Kit, Illumina), and size selected from 200 to 800 bp (BluePippin, Sage Sciences), following manufacturer's recommendations.

### ATAC-Sequencing Analysis

Before starting the alignment and downstream analysis, quality check was done by FastQC (https://tumor.bioinformatics.babraham.ac.uk/projects/fastqc/). Adaptors sequences were removed with TrimmomaticPE using options “HEADCROP: 10 CROP:70 ILLUMINACLIP: adaptor. file:2:30:10 LEADING:3 TRAILING:3 SLIDINGWINDOW:4:15 MINLEN:60” (Bolger, A. M., Lohse, M. & Usadel, B. Trimmomatic: a flexible trimmer for Illumina sequence data. Bioinformatics, 2014, vol. 30, 2114-2120, doi:10.1093/bioinformatics/btu170). ATAC-seq paired-end reads were then aligned to mouse genome Grcm38 using Bowtie2 (version 2.2.6) (Langmead, B. & Salzberg, S. L. Fast gapped-read alignment with Bowtie 2. Nat Methods, 2012, vol. 9) using options “-X 2000 --fr --very-sensitive --no-discordant --no-unal --no-mixed -non-deterministic”. Mitochondrial reads, reads from unmapped or random contigs and reads with a mapping quality less than 20 were removed using samtools (Li, H. et al. The Sequence Alignment/Map format and SAMtools. Bioinformatics, 2009, vol. 25). Duplicate reads were removed by Picard tools (http://broadinstitute.githubio/picard/).

Peak calling was performed on each individual sample by macs2 (version 2.1.0.20151222) (Zhang, Y. et al. Model-based analysis of ChIP-Seq (MACS). Genome Biol, 2008, vol. 9, R137) using options “-f BAMPE -g mm -q 0.05 --nomodel --call-summits -B -SPMR”. Peaks from the different subpopulations were merged for downstream analysis.

Reads counts of each merged peaks for each individual sample were calculated by HTSeq-count (Heinz, S. et al. Simple combinations of lineage-determining transcription factors prime cis-regulatory elements required for macrophage and B cell identities. Mol Cell, 2010, vol. 38, 576-589) using options “-f bam -r pos -m intersection-nonempty”. These counts were normalized for one million of mapped reads in merged peaks and fold of change were calculated between the subpopulations. Peaks were associated to genes with GREAT software (McLean, C. Y. et al. GREAT improves functional interpretation of cis-regulatory regions. Nat Biotechnol, 2010, vol. 28) with the following parameters: 5.0 kb in proximal upstream, 1.0 kb in proximal downstream and 100.0 kb in distal. For most of the analysis, only peaks annotated to at least one gene were kept.

Differential peaks are defined as peaks having at least a 2-fold change between the 2 subpopulations and being called peak in the subpopulation were they are higher.

De novo motif search was performed using findMotifsGenome.pl program in HOMER software (Anders, S., Pyl, P. T. & Huber, W. HTSeq—a Python framework to work with high-throughput sequencing data. Bioinformatics, 2015, vol. 31, 166-169) using parameters “-size −250,250 -S 15 -len 6,8,10,12,16.” Specific motif research was performed using annotatePeaks.pl program in HOMER software using parameters “-size −250,250”.

### Analysis of the Predictive Value of Fat1 Signature in Human Cancers (TCGA Database)

RNA-sequencing raw counts from the lung squamous cell carcinoma (LUSC) datasets, disclosed by the The Cancer Genome Atlas (TCGA) consortium, were downloaded from the National Cancer Institute portal at https://portal.gdc.cancer.gov/. Raw counts calculated from HTSeq (Anders, S., et al. HTSeq—a Python framework to work with high-throughput sequencing data. Bioinformatics, 2015, vol. 31, 166-169) were normalized into reads per kilobase per million (RPKM) (Robinson, M. D., et al. edgeR: a Bioconductor package for differential expression analysis of digital gene expression data. Bioinformatics, 2010, vol. 26, 139-140) in order to consider the library size specific to each sample.

Common Fat1cKO signature defines as genes commonly upregulated (Fold Change 2) in mouse skin Fat1cKO SCC vs control, mouse lung Fat1cKO SCC vs control and in human FAT1 KO SCC cells vs FAT1 WT. The signature was calculated as a weighted sum of the log-expressions of the selected genes, with gene-specific weights equal to +1 or −1 depending on the direction of their association with the Fat1cKO Epcam+vs Control Epcam+. The signature was then scaled so that the 2.5% and 97.5% quantiles equaled −1 and +1, respectively. Associations between Fat1cKO mouse signature with overall survival (OS) were evaluated using Cox proportional hazards regression model (km.coxph.plot function from the R package genefu, version 2.8.0) (Gendoo, D. M. et al. Genefu: an R/Bioconductor package for computation of gene expression-based signatures in breast cancer. Bioinformatics, 2016, vol. 32, 1097-1099).

Association between FAT1 mutation (non-synonymous mutation with variant allele frequency, VAF, ≥20%) with the mutated FAT1 signature was assessed through Wilcoxon rank sum tests. Spearman correlation was used to evaluate the association between FAT1 VAF and the mutated FAT1 signature. Association between the mutated FAT1 signature and overall survival (OS) was evaluated using the Cox proportional hazard model and the log-rank test P value reported on Kaplan-Meier survival curves (Vasioukhin, V., et al. The magical touch: genome targeting in epidermal stem cells induced by tamoxifen application to mouse skin. Proc Natl Acad Sci USA, 1999, vol. 96, 8551-8556).

All analyses were performed on the R platform (version 3.4.4). All statistical analyses were performed as two-sided.

### ChIP-qPCR Experiments

FAT1 WT and FAT1 KO A388 cells were crosslinked for 10 min at room temperature with 1% formaldehyde in serum free medium. The reaction was quenched by addition of 0.125M glycine and washed twice with cold PBS. ChIP experiments were performed according to the ChIP-IT Express kit (Active Motif) protocol. Briefly, sonication was performed with a bioruptor (Diagenode) to produce chromatin fragments of an average of 300 bp. Five micrograms of rabbit monoclonal antibody for H3K27me3 (C36B11 Rabbit mAb, #9733 Cell Signaling Technologies) were incubated with bug chromatin overnight at 4 C. After extensive washing steps, DNA was eluted and reverse-crosslinked overnight at 65 C, then purified using the iPurev2 kit (Diagenode). One microliter of enriched DNA, 0.5 of primers and SYBR Green master mix was subjected to 45 cycles of PCR using LightCycler 480 II (Roche). The fold change of H3K27me3 value for the same genomic region in WT and FAT1KO cells was calculated based on the AA Ct of the IP in each cell line after input normalization. P-values were calculated using two-tailed one-sample t-test. An IgG control has been included to account for background chromatin binding (not shown). The primers for ChIP-qPCR of the methylated regions in the human Sox2 promoter and for the Control regions (EZH2/H3K27me3 negative control regions) have been obtained from published literature (Kushwaha, R. et al. Mechanism and Role of SOX2 Repression in Seminoma: Relevance to Human Germline Specification. Stem Cell Reports, 2016, vol. 6, 772-783; Torigata, K. et al. LATS2 Positively Regulates Polycomb Repressive Complex 2. PLoS One, 2016, vol. 11, e0158562).

### Sample Preparation for Proteomic Analysis

A total of 12 samples was prepared for LC-MS/MS analyses, corresponding to 3 replicates of 2 FAT1 WT and 2 CRISPR/Cas9 FAT1 KO clones of human A388 SCC cell line. Cell pellets (5 million cells per pellet) were lysed in a urea lysis buffer containing 8 M urea, 20 mM HEPES pH 8.0 and PhosSTOP phosphatase inhibitor cocktail (Roche, 1 tablet/10 ml buffer). The samples were sonicated with 3 pulses of 15 s at an amplitude of 20% using a 3 mm probe, with incubation on ice for 1 minute between pulses. After centrifugation for 15 minutes at 20,000×g at room temperature to remove insoluble components, proteins were reduced by addition of 5 mM DTT and incubation for 30 minutes at 55° C. and then alkylated by addition of 10 mM iodoacetamide and incubation for 15 minutes at room temperature in the dark. The protein concentration was measured using a Bradford assay (Bio-rad) and from each sample 1 mg protein was used to continue the protocol. Samples were further diluted with 20 mM HEPES pH 8.0 to a final urea concentration of 4 M and proteins were digested with 5 μg LysC (Wako) (1/200, w/w) for 4 hours at 37° C. Samples were again diluted to 2 M urea and digested with 5 μg trypsin (Promega) (1/200, w/w) overnight at 37° C. The resulting peptide mixture was acidified by addition of 1% trifluoroacetic acid (TFA) and after 15 minutes incubation on ice, samples were centrifuged for 15 minutes at 1,780×g at room temperature to remove insoluble components. Next, peptides were purified on SampliQ SPE C18 cartridges (Agilent). Columns were first washed with 1 ml 100% acetonitrile (ACN) and pre-equilibrated with 3 ml of solvent A (0.1% TFA in water/ACN (98:2, v/v)) before samples were loaded on the column. After peptide binding, the column was washed again with 2 ml of solvent A and peptides were eluted twice with 700 μl elution buffer (0.1% TFA in water/ACN (20:80, v/v)). Phosphopeptides were enriched with MagReSyn® Ti-IMAC beads following the protocol according to the manufacturer's instructions with slight modifications. Briefly, 100 μl MagReSyn® Ti-IMAC beads (per sample) were washed twice with 70% EtOH, once with 1% NH₄OH and three times with a mixture of water/ACN/TFA (14:80:6, v/v/v). Next, the digested sample was incubated with the washed beads for 30 min at room temperature, the beads were washed once with a mixture of water/ACN/TFA (14:80:6, v/v/v) and three times with a mixture of water/ACN/TFA (19:80:1, v/v/v). Phosphopeptides were eluted from the beads by adding three times 80 μl 1% NH₄OH. 60 μl 10% formic acid (FA) was added to the combined eluate and the samples were dried completely in a speedvac vacuum concentrator.

### LC-MS/MS Analysis

Peptides resulting from phosphopeptide enrichment were re-dissolved in 20 μl solvent A and 15 μl were injected for LC-MS/MS analysis on an Ultimate 3000 RSLCnano system in-line connected to a Q Exactive HF mass spectrometer equipped with a Nanospray Flex Ion source (Thermo). Trapping was performed at 10 μl/min for 4 min in solvent A on a 20 mm trapping column (made in-house, 100 μm internal diameter (I.D.), 5 um beads, C18 Reprosil-HD, Dr. Maisch, Germany) and the sample was loaded on a 200 cm long micro pillar array column (PharmaFluidics) with C18-endcapped functionality mounted in the Ultimate 3000's column oven at 50° C. For proper ionization, a fused silica PicoTip emitter (10 μm inner diameter) (New Objective) was connected to the μPAC™ outlet union and a grounded connection was provided to this union. Peptides were eluted by a non-linear increase from 1 to 55% MS solvent B (0.1% FA in water/ACN (2:8, v/v)) over 116 minutes, first at a flow rate of 750 nl/min, then at 300 nl/min, followed by a 14-minutes wash reaching 99% MS solvent B and re-equilibration with MS solvent A (0.1% FA in water). The mass spectrometer was operated in data-dependent mode, automatically switching between MS and MS/MS acquisition for the 8 most abundant ion peaks per MS spectrum. Full-scan MS spectra (375-1500 m/z) were acquired at a resolution of in the orbitrap analyser after accumulation to a target value of 3,000,000. The 8 most intense ions above a threshold value of 8,300 were isolated (window of 1.5 Th) for fragmentation at a normalized collision energy of 28% after filling the trap at a target value of 100,000 for maximum 120 ms. MS/MS spectra (200-2000 m/z) were acquired at a resolution of 15,000 in the orbitrap analyser. The S-lens RF level was set at 50 and we excluded precursor ions with single, unassigned and >7 charge states from fragmentation selection. QCloud was used to control instrument longitudinal performance during the project (Chiva, C. et al. QCloud: A cloud-based quality control system for mass spectrometry-based proteomics laboratories. PLoS One, 2018, vol. 13, e0189209).

### Phosphoproteomic Data Analysis

Data analysis of the phosphoproteomics data was performed with MaxQuant (version 1.6.3.4) using the Andromeda search engine with default search settings including a false discovery rate set at 1% on PSM, peptide and protein level. Spectra were searched against the human proteins in the Swiss-Prot Reference Proteome database (database release version of June 2019 containing 20,960 human protein sequences, (http://www.uniprot.org). The mass tolerance for precursor and fragment ions was set to 4.5 and 20 ppm, respectively, during the main search. Enzyme specificity was set as C-terminal to arginine and lysine, also allowing cleavage at proline bonds with a maximum of two missed cleavages. Variable modifications were set to oxidation of methionine residues, acetylation of protein N-termini and phosphorylation of serine, threonine or tyrosine residues, while carbamidomethylation of cysteine residues was set as fixed modification. Matching between runs was enabled with a matching time window of 0.7 minutes and an alignment time window of 20 minutes. Only proteins with at least one unique or razor peptide were retained leading to the identification of 7,856 phosphorylated sites. Proteins were quantified by the MaxLFQ algorithm integrated in the MaxQuant software. A minimum ratio count of two unique or razor peptides was required for quantification.

For the analysis of the phosphoproteomics data, the phospho(STY)sites file was loaded in the Perseus software (version 1.6.2.1). Reverse hits were removed, the site table was expanded, the intensity values were log 2 transformed and the median was subtracted. Replicate samples were grouped, phosphosites with less than three valid values in at least one group were removed and missing values were imputed from a normal distribution around the detection limit leading to a list of 3311 quantified phosphopeptides that was used for further data analysis. Then, a t-test was performed (FDR=0.05 and s0=1) to compare control and KO samples and a volcano plot was generated. 288 phosphopeptides were significantly upregulated in FAT1 KO samples and 335 phosphopeptides were significantly upregulated in FAT1 WT samples, and plotted in a heatmap after non-supervised hierarchical clustering.

### Quantification and Statistical Analysis

Two-tailed t-student, two-tailed Mann Whitney U and survival analysis (Kaplan-Maier) were performed using GraphPad Prism version 7.00 for Mac, GraphPad Software, La Jolla California USA, tumor.graphpad.com.

The statistical p value for TPC was obtained using a Chi-square test. The statistical p values for the number of metastasis and the proportion of the different subpopulations in skin and metaplastic breast TUMORS were calculated using t-test.

Other statistical methods: Dendrogram for clustering of subpopulations (RNA-seq and ATAC-seq data) was drawn on the number of reads in the 500 annotated merged peaks with highest variance across the samples using canberra distance and complete clustering method thanks to heatmap.2 function in R software (Foundation for Statistical Computing, Vienna, Austria) (http://tumor.bioconductor.org/).

To estimate if the proportion of peaks annotated to genes having a smad2 binding site for all the different comparisons of EMT and MET was higher than in the whole genome present inventors randomly generated 5 sets of 10000 peaks of 500 bp in the whole mouse genome with RSAT software (Medina-Rivera, A. et al., RSAT 2015: regulatory sequence analysis tooln Nucleic Acids Res, 2015, vol. 43, W50-W56). SMAD2 binding sites were searched with HOMER exactly as in peaks for EMT leading to 2832, 2788, 2782, 2764 and 2797 peaks among the 10000 having at least one potential binding for SMAD2. Present inventors took 2793 peaks as the average. The prop.test function of the R software was used with alternative=“greater” parameter to test if the proportion was higher.

All the statistical analyses are based on biological replicates (that correspond to “n” indicated in the text, figures or figure legends). No technical replicates were used to calculate statistics.

### Example 2. Key Signaling Pathways, Genes and Transcription Factors Associated with Fat1 Mutated Tumors

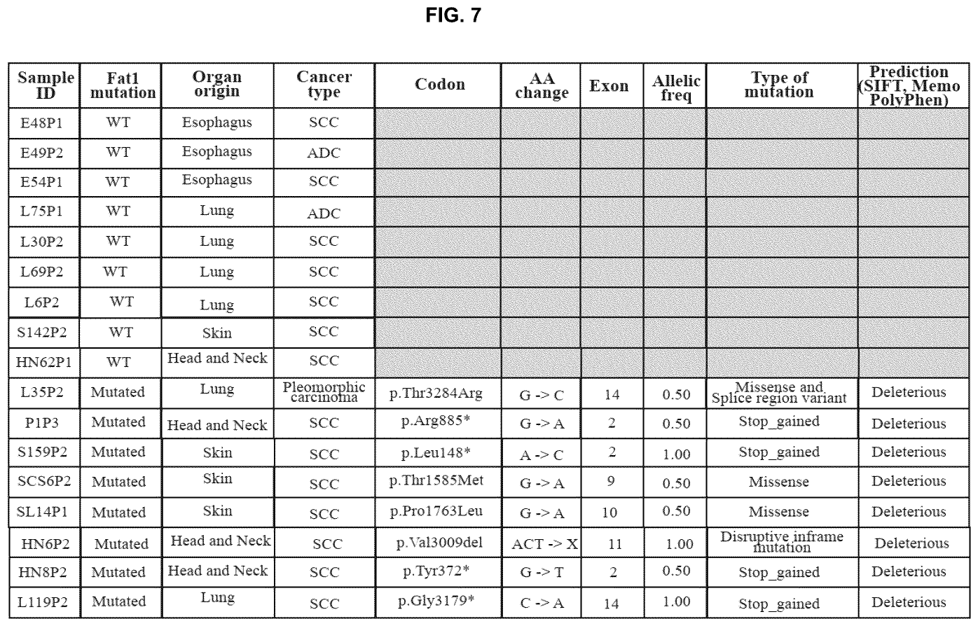

Present inventors demonstrated that FAT1 loss-of-function (LOF), one of the most frequent tumor suppressor genes, promotes tumor initiation and progression in vivo. Furthermore, present inventors found that FAT1 LOF promotes the acquisition of a hybrid epithelial-mesenchymal transition (EMT) states presenting increased tumor stemness and metastasis. For example, present inventors found that FAT1 mutated SCCs exhibit a much higher EMT hybrid score as compared to FAT1 WT SCCs. The FAT1 mutations in the SCCs are shown in FIG. 7. To investigate the molecular mechanisms by which Fat1 LOF promotes hybrid EMT state, present inventors explored the transcriptional and chromatin landscape of Epcam+ and Epcam− tumor cells (TCs) from WT and Fat1cKO mouse skin SCCs. Present inventors demonstrated that Epcam+ Fat1cKO TCs are transcriptionally primed to undergo EMT during tumorigenesis, and following EMT, Fat1cKO Epcam− TCs are stabilized in a hybrid EMT state associated with poor survival in lung cancer. More particularly, present inventors found that high expression of the common RNA mesenchymal signature between mouse skin and lung Fat1cKO SCC and human FAT1 KO SCC was associated with poor overall survival in patients with lung SCC and that this common Fat1 signature was associated with the presence of FAT1 point mutations with variant allelic frequency of more than 20% and with FAT1 deletion (FIG. 2 a-b).

### Yap1 and Sox2 Regulate Mesenchymal and Epithelial States Downstream of Fat1 Deletion

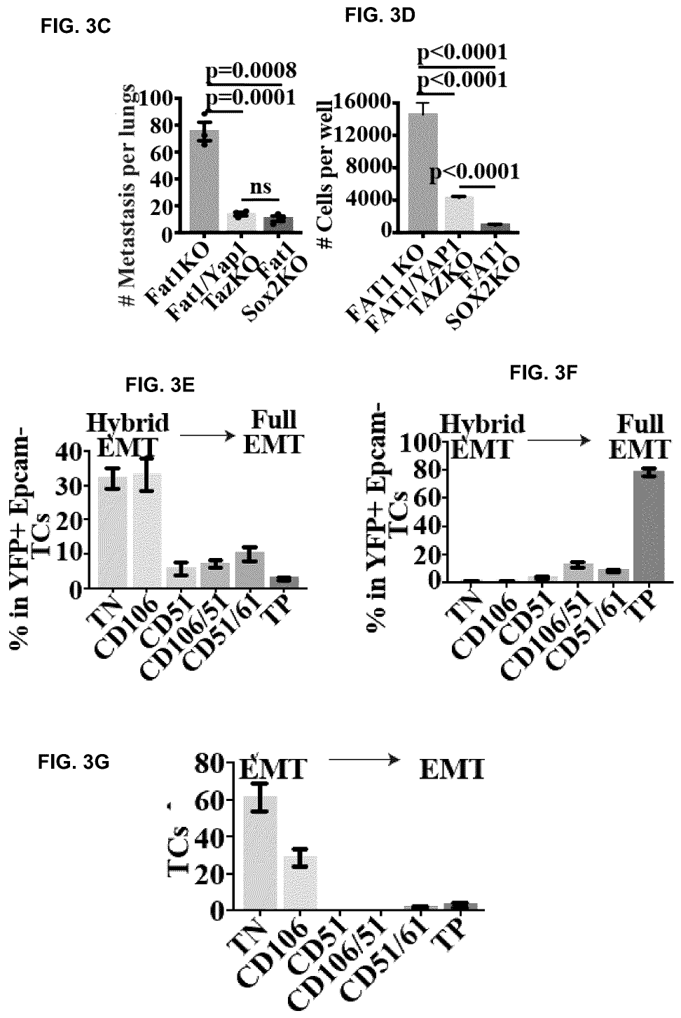

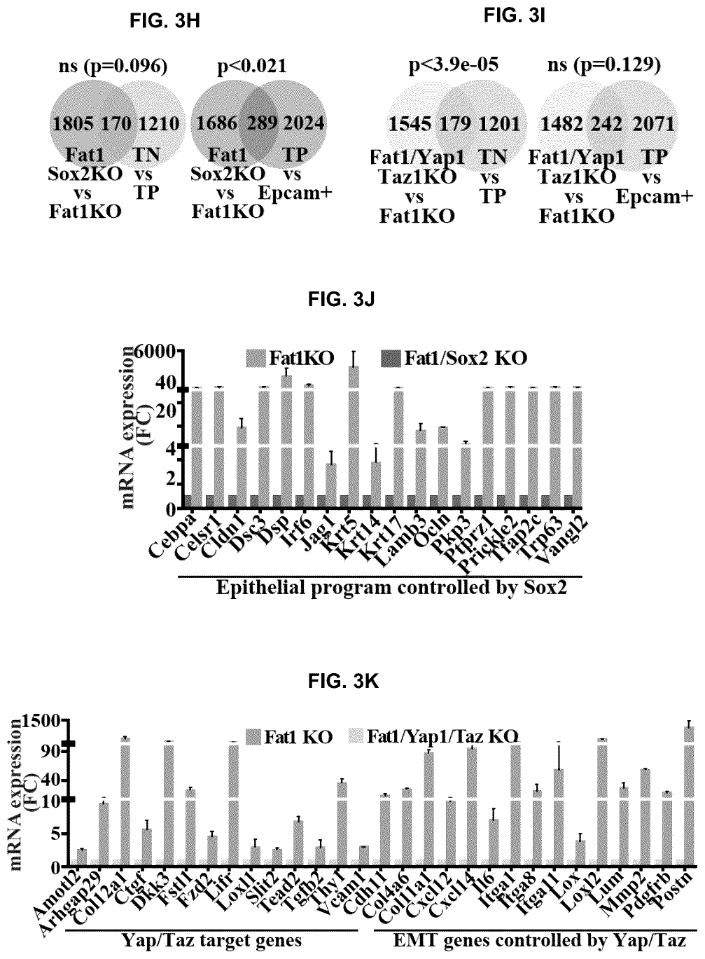

To define the changes in the chromatin landscape responsible for the transcriptional priming of hybrid EMT state that occurs following Fat1 deletion, present inventors performed ATAC-seq on FACS-isolated WT and Fat1cKO Epcam+ and Epcam− TC populations. Present inventors found enhancers within key EMT TFs such as Zeb 1, Snail1 and Twist2 and other EMT markers (eg: Vim, Col6a3, and others) that are already more accessible in Epcam+ Fat1 cKO TCs as compared to WT Epcam+ TCs, potentially accounting for the epigenetic priming of EMT upon Fat1 deletion. Present inventors found enhancers that get opened only in Epcam− Fat1cKO as compared to Epcam+, and which are associated with progression in EMT, such as in the regulatory regions of Prrx1, Nfatc1, Pdgfrb or Vcam1/CD106. To define the TFs responsible for the chromatin priming of EMT in Epcam+ Fat1cKO cells, present inventors performed motif discovery analysis of the ATAC-seq peaks that were more opened in Epcam+ Fat1cKO TCs as compared to Epcam+WT TCs. Present inventors found that motifs for AP1, Nfi, Runx, Mafk, Tead, Nfkb TFs were strongly statistically enriched in the chromatin regions of Fat1cKO Epcam+ TCs, suggesting that the Jun/Fos family of TFs cooperates with other TFs including those of the TEAD family, that relay the Yap1 pathway to the nucleus (FIG. 3a) to prime the Fat1 mutated cancer cells to undergo EMT in skin SCC in vivo.

To define the chromatin regions responsible for the hybrid EMT states and for the sustained expression of some epithelial genes in Epcam− Fat1cKO TCs, present inventors investigated the chromatin landscape of the regulatory regions of pro-epithelial TFs. Consistent with their expression changes, present inventors found that many enhancers located in the regulatory regions of pro-epithelial TFs such as Cebpa, Cebpb, Grhl1, Sox2, Klf4 or AP2g were still opened in Epcam− Fat1cKO TCs, whereas these enhancers were completely closed in Epcam− TCs from Epcam− TCs from Lgr5CREER/KRas G12D/p53^{cKO} SCCs. Immunostaining confirmed the sustained expression of these pro-epithelial TFs (Sox2, p63, or Klf4) in Fat1 cKO hybrid EMT cells. To identify the TFs responsible for maintaining the hybrid epithelial phenotype, present inventors performed motif discovery analysis in the ATAC-seq peaks that were upregulated in Epcam− Fat1cKO TCs as compared to Epcam− control TCs from fully mesenchymal Lgr5CREER/KRas^{G12D}/p53^{cKO} SCCs. Present inventors found that AP1, Sox, Klf, Lhx and MafK motifs were strongly statistically enriched in Epcam− Fat1 cKO TCs (FIG. 3a), suggesting that the epithelial program of the hybrid EMT state in Fat1cKO is mediated by an AP1/Sox2/Klf transcriptional network. The AP1 TF can induce chromatin remodelling in Ras driven cancers at both epithelial and mesenchymal enhancers. YAP1/Tead cooperate with AP1 to promote skin tumor initiation and EMT. SOX2 is amplified in many human SCCs and marks cancer stem cells in skin SCCs, and could be responsible for the hybrid EMT state and the sustained expression of epithelial genes in Fat1cKO TCs.

To functionally validate the bio-informatic prediction of the gene regulatory network that controls the hybrid EMT state in Fat1 deficient tumors, present inventors assessed the impact of CRISPR/Cas9 mediated deletion of Yap1/Taz and Sox2 on tumor stemness, metastasis and gene expression program of mouse skin SCCs. Limiting dilution transplantation of Sox2 KO and Yap1/Taz KO primary Epcam negative cell lines derived from Lgr5CREER/KRas^{G12D}/p53^{cKO}/Fat^{cKO} SCCs displayed a decreased TPC frequency in both cases (FIG. 3b). The number of metastasis was also importantly reduced upon deletion of either Sox2 or Yap1/Taz (FIG. 3c), demonstrating that Sox2 and Yap1/Taz transcriptional programs are important for the promotion of tumor stemness and metastasis downstream of Fat1 deletion in mouse tumors. To assess the human relevance of these findings, present inventors performed CRISPR/Cas9 deletion of either SOX2 or YAP1/TAZ in FAT1 deleted human SCC cell lines. SOX2 or YAP1/TAZ KO decreased the promotion of tumor growth mediated by FAT1 deletion in 3D spheroid assays (FIG. 3d), demonstrating that SOX2 and YAP1/TAZ promote tumor growth downstream of FAT1 in human cancer cells. Conversely, the deletion of E-cadherin in the same cell line, which induced defects of cell adhesion, did not promote tumor growth and did not induce SOX2 or ZEB1 expression, or increase in nuclear YAP1.

To assess the respective role of Sox2 and Yap1/Taz in the transcriptional program mediated by Fat1 deletion, present inventors assessed the FACS profile, histology, expression of epithelial and mesenchymal markers and transcriptional profile in Fat1 mutated tumors presenting simultaneous deletion of Sox2 or Yap1/Taz following their transplantation into immunodeficient mice. Deletion of Sox2 in Lgr5CREER/KRas^{G12D}/p53^{cKO}/Fat^{cKO} SCCs resulted in the loss of epithelial characteristics and a shift from hybrid to complete EMT, as shown by immunostaining (complete loss of Krt14) and FACS analysis (shift from hybrid to late EMT TP EMT subpopulation) (FIG. 3e-f). The RNA-seq of Fat1/Sox2 double KO further demonstrated a significant enrichment in late EMT signature (FIG. 3h), marked by an increase of mesenchymal markers (e.g. Lox, Col4a6, Mmp9, Pdgfra) and a decrease of epithelial markers (e.g. Cebpa, Krt5, Trp63) (FIG. 3j and data not shown).

Instead, the deletion of Yap/Taz promoted early hybrid EMT state as shown by an enrichment of Epcam-Triple Negative (TN) population and an absence of TCs presenting late EMT stages (FIG. 3g).

The transcriptome of Fat1/Yap1/Taz triple KO showed significant enrichment of Epcam+ epithelial and early hybrid EMT (TN) signature (FIG. 3i). Among the genes downregulated in Fat1/Yap1/Taz triple KO, present inventors found that many classical canonical Yap1/Taz target genes (e.g. Ctgf, Amotl2, Fstl1) as well as many other genes associated with EMT (eg: Vcam1, Thy1, Pdfrb), were decreased compared to Fat1 LOF (FIG. 3k). Altogether these data demonstrate that Sox2 and Yap1/Taz control distinct transcriptional programs leading to a stable hybrid EMT phenotype downstream of Fat1 LOF.

### Phosphoproteomic Analysis Identifies the Signalling Cascades Downstream of FAT1 Deletion

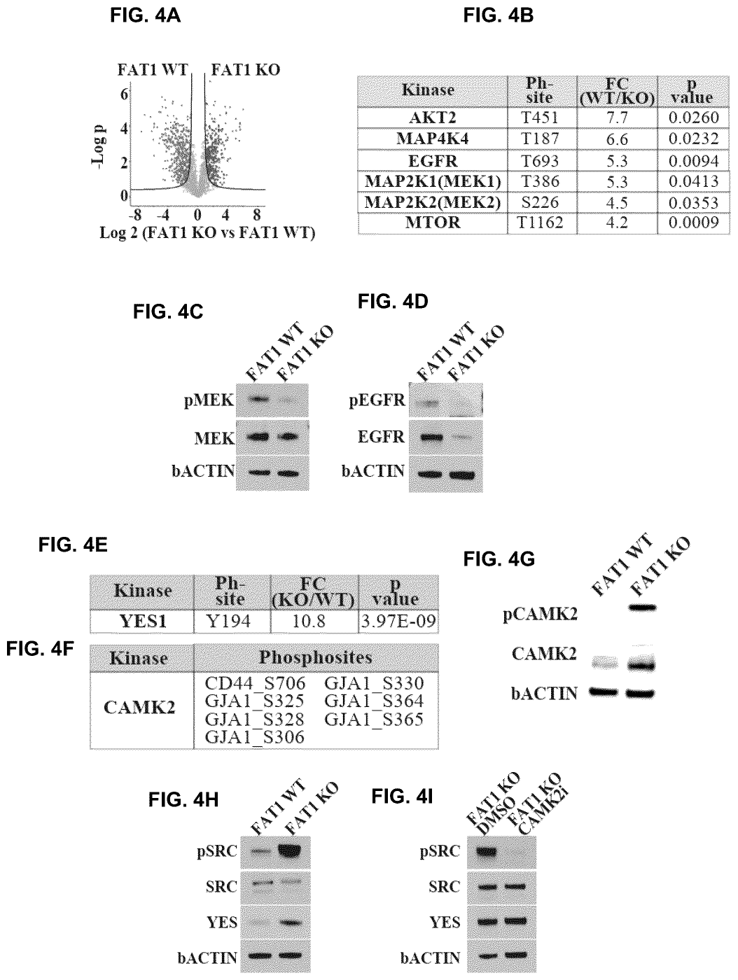

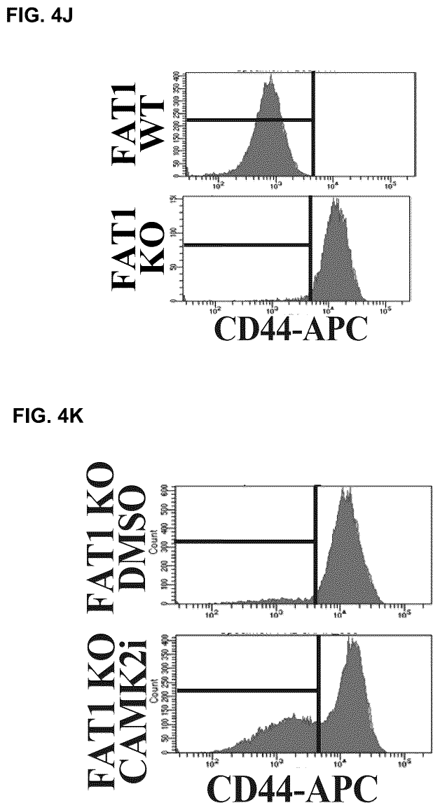

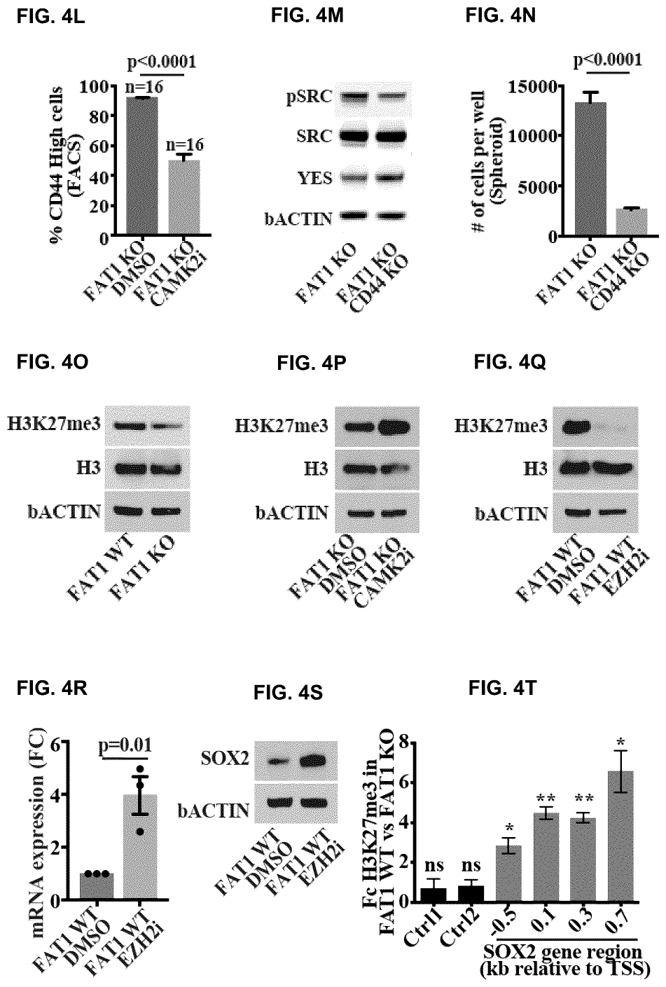

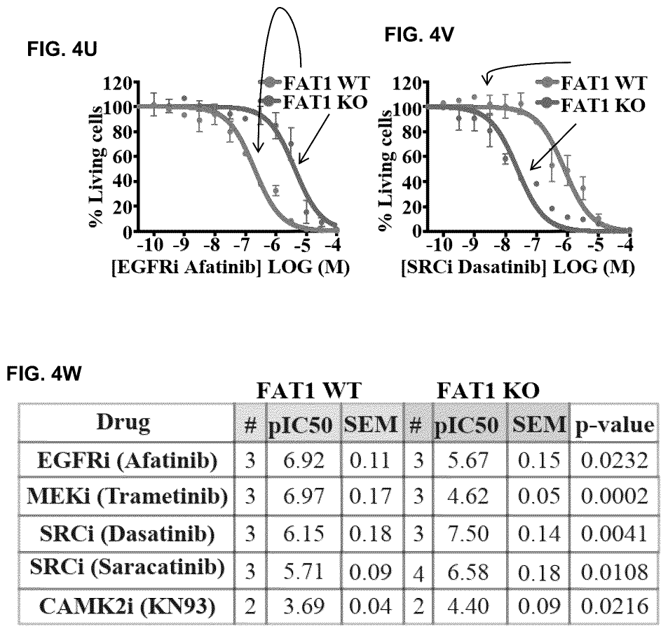

To understand how the deletion of Fat1, a protocadherin which is located at the plasma membrane, leads to the activation of a transcriptional program controlled by Sox2 and Yap1/Taz, present inventors performed phosphoproteomic analysis of WT and CRISPR/Cas9 FAT1 KO human SCC cells, to dissect step by step the signaling pathways that are activated downstream of FAT1 deletion. Present inventors quantified the phosphopeptides that were differentially regulated between WT and FAT1 deleted isogenic human SCC cell lines. It was found that 288 phosphosites were significantly upregulated and 335 were significantly downregulated in FAT1 KO TCs as compared to FAT1 WT (FIG. 4a).

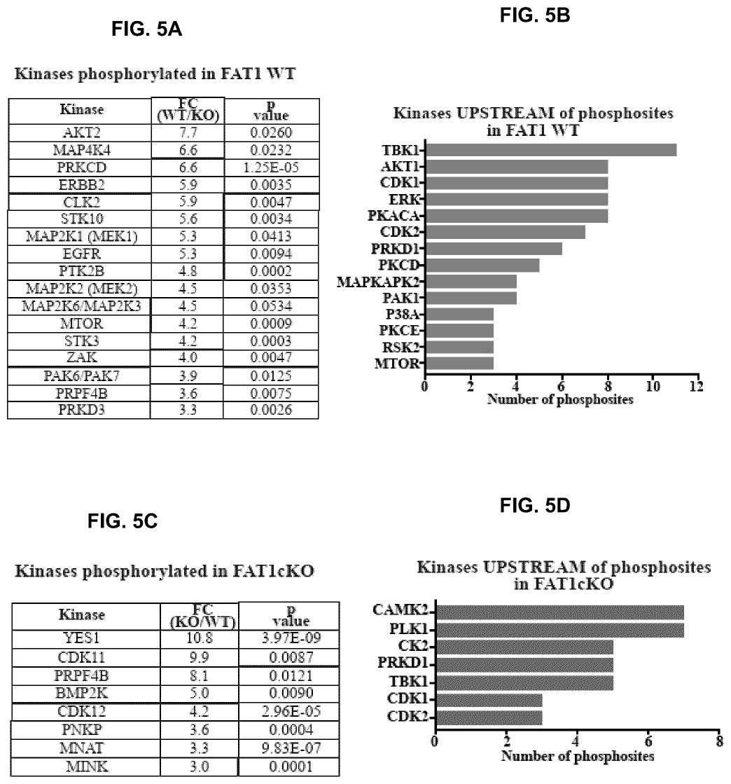

Upon FAT1 LOF a massive decrease in the phosphorylation of the proteins involved in cell-cell adhesion was observed, such as ZO-1 (S1617) and ZO-2 (S130, S131, S1159, S986, S226). MAP4K4, that has been reported to phosphorylate LATS1, inhibiting YAP1 and decreasing YAP1-TEAD4 interaction, was among the most strongly upregulated kinases in FAT1 WT TCs (FIG. 4b). In addition, present inventors identified PRKCD (S304), EGFR (T693), ERBB2 (S998), MEK1 (T386), MEK2 (S226), AKT2 (phosphorylated on T451) and MTOR (T1162) among the proteins that were significantly more phosphorylated in FAT1 WT TCs (FIG. 4b, FIG. 5 a, b). In good accordance with the phosphoproteomic analysis and validating the present inventors' prediction about the pathways downregulated upon FAT1 LOF, MEK was significantly more phosphorylated and the total levels of EGFR and pEGFR were increased in FAT1 WT TCs (FIG. 4 c, d). These data suggest that the activity of the EGFR-RAS-RAF-MEK-MAPK and of the EGFR-PI3K-AKT-MTOR signaling pathways is decreased upon FAT1 LOF.

Conversely, FAT1-deficient TCs exhibited strong increase in the phosphorylation on Y194 of proto-oncogene tyrosine-protein kinase YES (FIG. 4e, FIG. 5c), that belongs to the SRC Family of Tyrosine Kinases, as well as the MAP1B (S1252, S1779, T948, T948, S2098, S937, S1298 and S1312) and GJA1 (S306, S328, S325, S330, T326, S365 and S364) proteins. GJA1 phosphorylation promotes the localization of GJA1 at the membrane and increases functional gap junction formation, which has been linked to increased metastatic capacity of TCs. These data suggest that FAT1 LOF induces a global remodelling of cell-cell adhesions, intercellular communications and cellular cytoskeleton leading to acquisition of hybrid EMT phenotypes.

To decipher the signalling cascade acting downstream of FAT1 LOF, the present inventors used PhosphoSitePlus online tool and bibliographic search to predict kinases acting upstream of identified phosphosites. Interestingly, Ca²⁺/Calmodulin-dependent protein kinase II (CAMK2) was most frequently found to act upstream of phosphopeptides enriched in FAT1cKO TCs (CAMK2 was predicted to phosphorylate CD44 on Serine 706, EZH2 on Threonine 487 and GJA1 on Serine 328, Serine 325, Serine 330, Serine 364 and Serine 365) (FIG. 4f, FIG. 5d). PLK1 was another kinase frequently found to be upstream of phosphopeptides enriched upon FAT1 LOF (FIG. 5d).

Then Western Blot (WB) was used to validate these findings and used small molecule inhibitors of the different kinases to define the functional relevance of the predicted kinase-protein networks. According to the bio-informatic prediction it was found that CAMK2 is significantly more phosphorylated upon FAT1 LOF (FIG. 4g). It was further confirmed that SRC/YES was also more expressed and phosphorylated upon FAT1 LOF (FIG. 4h) and that CAMK2 inhibitor (KN93) greatly decreased SRC/YES phosphorylation levels (FIG. 4i), showing that CAMK2 directly or indirectly phosphorylates YES/SRC upon FAT1 LOF.

CD44 is a protein previously reported to be upregulated during EMT, and to promote tumor stemness, tumor progression and metastasis. Computational analysis predicted an ESRP1-CD44-ZEB1 loop to stabilize hybrid EMT state in human lung cancer cells. Phosphorylation regulates CD44 cellular localization and signaling. Present inventors found that phosphorylation on S706 was upregulated upon FAT1 LOF (FIG. 4f). FACS analysis revealed that FAT1 KO cells expressed higher levels of surface CD44 (FIG. 4j). To understand whether CAMK2 is responsible for the stabilization of CD44 on the membrane, present inventors treated FAT1 KO cells with CAMK2 inhibitor and observed that the levels of surface CD44 decreased significantly upon CAMK2 inhibition in FAT1 KO cells (FIGS. 4 k, l). Importantly, CD44 signaling has been shown to promote the phosphorylation of SRC. To determine whether CAMK2 phosphorylates YES/SRC directly or through CD44 signaling in FAT1 KO cells, CRISPR/Cas9 CD44 deletion in FAT1 KO cells was performed and it was found that pSRC was decreased upon CD44/FAT1 double KO (FIG. 4m). These data demonstrate that upon FAT1 LOF, CAMK2 activates SRC at least partially though CD44. The clonogenicity of FAT1/CD44 KO human SCC cells decreased significantly in 3D tumor spheroid assays (FIG. 4n), demonstrating that CD44 stabilization contributes to the increase in tumor stemness observed upon FAT1 LOF.

Then present inventors assessed whether the hybrid EMT phenotype could be explained, at least in part, by CAMK2-SRC signaling. For that purpose, the expression of YAP1, ZEB1, E-Cadherin and SOX2 was analyzed in FAT1 KO, FAT1/CD44 KO cells and FAT1 KO cells treated with CAMK2 or SRC-inhibitors. Present inventors found that FAT1/CD44 KO tumor cells, FAT1 KO cells treated with CAMK-inhibitor (KN93) and FAT1 KO cells treated with SRC-inhibitor (Saracatinib or Dasatinib) presented a strong decrease in nuclear YAP1 and ZEB1, an increase in E-Cadherin expression and were growing in more compact epithelial colonies (data not shown). These results demonstrated that FAT1 LOF activates a CAMK2/CD44/SRC-YAP/ZEB1 axis that promotes the expression of a mesenchymal program. Present inventors observed a decrease in SOX2 expression in FAT1 KO TCs treated with CAMK2 inhibitor. However, no change in SOX2 was observed upon inhibition of the CD44/SRC cascade (data not shown).

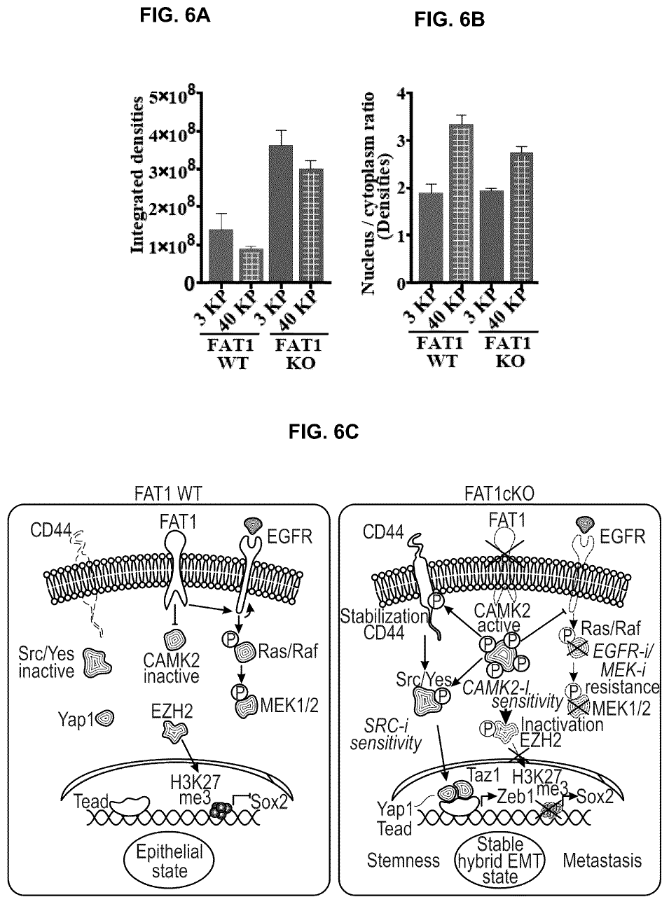

The phospho-proteomic analysis of present inventors revealed that EZH2 was significantly more phosphorylated on Thr487 in FAT1 KO cells as compared to FAT1 WT cells. This phosphorylation site has been reported to inactivate EZH2. EZH2 is a key part of the PRC2 complex that methylates H3 at Lys 27, mediating transcriptional repression. Present inventors have previously found that this histone mark is remodeled at the Sox2 locus during SCC formation. Present inventors hypothesized that EZH2 inhibition in FAT1 KO cells could decrease H3K27me3 repressive histone marks and thus promote the expression of SOX2 transcriptionally. The global level of H3K27me3 was significantly decreased in FAT1 KO cells (FIG. 4o), supporting the notion that EZH2 could be less active upon FAT1 LOF. When FAT1 KO cells were treated with CAMK2 inhibitor, the global levels of H3K27me3 increased (FIG. 4p), consistently with the notion that CAMK2 activation inhibits EZH2/PRC2 activity in TCs. To functionally assess whether EZH2 inhibition leads to a decrease in H3K27me3 marks and increased SOX2 mRNA expression, FAT1 WT cells were treated with an EZH2 inhibitor (GSK343). H3K27me3 was decreased and SOX2 mRNA and protein expression were increased 7 days following EZH2 inhibitor treatment in FAT1 WT cells (FIG. 4 q, r, s), further suggesting that SOX2 is epigenetically regulated by a FAT1/CAMK2/EZH2 dependent mechanism. To further demonstrate that FAT1 deletion decreases the repressive histone marks at the SOX2 locus, present inventors assessed the deposition of the H3K27me3 repressive mark in the SOX2 promoter region in the presence or absence of FAT1. ChIP-qPCR showed that H3K27me3 marks around the SOX2 promoter were significantly reduced upon FAT1 deletion, supporting the notion that FAT1 deletion regulates the expression of SOX2 through an epigenetic mechanism (FIG. 4t). As YAP/TAZ signaling has been shown to be regulated by the stiffness of the extracellular matrix, present inventors assessed whether changes in stiffness of the matrix could be responsible, at least in part, for YAP1 nuclear localization or SOX2 expression upon FAT1 LOF. To this end, present inventors cultured FAT1 WT and FAT1 KO on coverslips coated with different stiffness conditions (3 and 40 kPa) and on glass. As expected, FAT1 WT cells presented very low nuclear YAP1 when cultured on low stiffness substrate (3KP) and high nuclear YAP1 when cultured on high stiffness substrate (40KP) or glass. Interestingly, FAT1 KO tumor cells exhibited high levels of total and nuclear YAP1 expression even on soft substrate. Higher stiffness further increased YAP1 nuclear localization (FIG. 6a-b). These data demonstrate that FAT1 loss of function constitutively activates signaling pathways leading to high YAP1 expression, causing the FAT1 KO cells to behave in respect to YAP1 nuclear expression as if tumor cells were exposed to stiff substrate. FAT1 WT cells were negative for SOX2 independently of the stiffness of the substrate, while FAT1 KO cells expressed high levels of SOX2 in all conditions, demonstrating that SOX2 is constitutively activated upon FAT1 LOF independently of the extracellular stiffness.

In summary, present inventors identified the epigenetic and transcriptional mechanisms that link loss of cell polarity and cell adhesion with the induction of a hybrid EMT phenotype downstream of Fat1 deletion. The present inventors' comprehensive molecular characterization including transciptomic, epigenomic, and proteomic characterization of Fat1 mutants show that the hybrid EMT signature is mediated by the activation of YAP1 and Sox2, which regulates respectively the co-expression of mesenchymal and epithelial transcriptional programs in cancer cells. Importantly, present inventors showed that gene signature associated with Fat1 loss of function is predictive of poor survival in lung cancer patients. Furthermore, using phosphoproteomic analysis coupled with functional characterization of protein phosphorylation network activated or inhibited upon FAT1 deletion, present inventors identified the signaling cascades leading to the activation of YAP1 and SOX2 downstream of Fat1 LOF. Fat1 deletion activates CAMK2, which induced the phosphorylation of SRC/YES and CD44, which promote YAP nuclear translocation and the induction of EMT program including ZEB1 expression. CAMK2 activation also lead to the phosphorylation of EZH2 at Thr487, which inhibits its activity and lead to a decrease of the chromatin repressive mark at SOX2 regulatory regions, which lead to SOX2 upregulation, sustaining the expression of the epithelial program. In addition, FAT1 deletion also decreases the activation of EGFR/MEK pathway.

### Example 3. Resistance to Neoplastic Agents and Vulnerabilities in FAT1 Mutated Tumors

The treatment of advanced SCCs from different organs, such as head and neck, oesophagus or lung, remains challenging, and the prognosis is particularly poor in metastatic disease. FAT1 is among the most frequently mutated genes in SCCs. To which extent FAT1 mutations impact the response to therapy in these cancers is currently unknown. Present inventors tested whether the signalling cascades changed upon FAT1 LOF could predict therapeutic resistance and vulnerability of these cancers. To test this hypothesis, present inventors assessed the sensitivity of WT and isogenic FAT1 KO human cancer cell lines to the inhibitors of the signalling pathways that were found to be differentially regulated between WT and FAT1 KO cells. EGFR inhibitors such as Afatinib are widely used in patients with metastatic SCC. MEK inhibitors also have been proposed to be an attractive therapeutic strategy in metastatic SCC. Very interestingly, FAT1 KO cells were significantly more resistant to the EGFR-inhibitor Afatinib and the MEK-inhibitor Trametinib as compared to FAT1 WT SCC cells (FIG. 4u-v).

In sharp contrast, FAT1 KO TCs were significantly more sensitive to the SRC inhibitors Dasatinib and Saracatinib and the CAMK2 inhibitor KN93 as compared to FAT1 WT TCs (FIG. 4v, w). These results indicate that the understanding of the molecular mechanisms underlying FAT1 LOF can lead to a much better targeted therapy in patients with FAT1-mutated cancers.

In summary, the activation and inhibition of the signaling pathways differentially regulated in FAT1 mutated tumor cells compared to FAT1 wild-type tumor cells lead to an increased sensitivity of FAT1 mutated cancer cells to CAMK2 and SRC inhibition and to resistance to EGFR and MEK inhibition (FIG. 6c). This study has important implications for personalized medicine and the prognosis and treatment of the high number of patients with cancer displaying FAT1 mutations.

### Example 4. Determining Sensitivity or Resistance of a Subject to Treatment with an Antineoplastic Agent and Treating the Subject

A biological sample (e.g. tissue biopsy) is obtained from a SCC patient (e.g. SCC of the skin, SCC of the head and neck, SCC of the oesophagus or SCC of the lung). Subsequently, a first part of the biological sample is prepared for sequence analysis and second part of the sample prepared for protein analysis.

The first part of the biological sample is subjected to genomic sequence analysis.

When the sequence analysis reveals the presence of one or more FAT1 loss-of-function mutations in the biological sample, it is decided to administer to the subject from which the biological sample is taken a CAMK inhibitor or a SRC kinase inhibitor for treating SCC. When no FAT1 loss-of-function mutation is detected in the biological sample, it is decided to administer to the subject from which the biological sample is taken an EGFR inhibitor or a MEK inhibitor.

The second part of the sample is subjected to FAT1 protein analysis (e.g. Western blot analysis).

When a reduced FAT1 protein level or no FAT1 protein is detected in the biological sample, it is decided to administer to the subject from which the biological sample is taken a CAMK inhibitor or a SRC kinase inhibitor for treating SCC. When increased or unchanged FAT1 protein levels are detected in the biological sample, it is decided to administer to the subject from which the biological sample is taken an EGFR inhibitor or a MEK inhibitor.

1. A method for determining sensitivity or resistance to treatment with an antineoplastic agent in a subject diagnosed with a neoplastic disease, the method comprising:
determining in a biological sample obtained from the subject, the presence or absence of a genetic or epigenetic alteration which reduces or abolishes expression or function of FAT1, or
determining that FAT1 expression or function is reduced or abolished in a biological sample obtained from the subject;
wherein the antineoplastic agent is selected from the group consisting of an epidermal growth factor receptor (EGFR) inhibitor, a mitogen-activated protein kinase (MEK) inhibitor, a Ca²⁺/calmodulin-dependent protein kinase (CAMK) inhibitor, and a SRC kinase inhibitor.

2. A method of treating a subject diagnosed with a neoplastic disease, comprising
determining the sensitivity or resistance of the subject to treatment with an antineoplastic agent by:
determining in a biological sample obtained from the subject the presence or absence of a genetic or epigenetic alteration which reduces or abolishes expression or function of FAT1, or
determining that FAT1 expression or function is reduced or abolished in a biological sample obtained from the subject,
wherein the antineoplastic agent is selected from the group consisting of an EGFR inhibitor, a MEK inhibitor, a CAMK inhibitor, and a SRC kinase inhibitor; and
treating the subject with the antineoplastic agent if the subject is determined to be sensitive to the antineoplastic agent.

3. The method according to claim 1, wherein the antineoplastic agent is an antineoplastic agent selected from the group consisting of an EGFR inhibitor, a MEK inhibitor, and a combination thereof, and wherein
the presence of the genetic or epigenetic alteration which reduces or abolishes expression or function of FAT1 in the biological sample, or
reduced or abolished expression or function of FAT1 in the biological sample indicates that the subject is resistant to treatment with the antineoplastic agent.

4. The method according to claim 1, wherein the antineoplastic agent is an antineoplastic agent selected from the group consisting of a CAMK inhibitor, a SRC kinase inhibitor, and a combination thereof, and wherein
the presence of the genetic or epigenetic alteration which reduces or abolishes expression or function of FAT1 in the biological sample, or
reduced or abolished expression or function of FAT1 in the biological sample indicates that the subject is sensitive to treatment with the antineoplastic agent.

5. The method according to claim 1, wherein the biological sample comprises neoplastic cells.

6. The method according to claim 1, wherein the presence or absence of the genetic or epigenetic alteration or the expression or function of FAT1 is determined by a technique selected from the group consisting of nucleic acid analysis, immunological assay, functional assay, and a combination thereof.

7. A kit for determining sensitivity or resistance to treatment with an antineoplastic agent, comprising
means for determining in a biological sample the presence or absence of a genetic or epigenetic alteration which reduces or abolishes expression or function of FAT1, or
means for determining FAT1 expression or function in a biological sample, and
a computer readable storage medium having recorded thereon one or more programs for carrying out the method of claim 1.

8. (canceled)

9. The method according to claim 1, wherein said neoplastic disease is of epithelial, mesenchymal or melanocyte origin, preferably of epithelial origin.

10. The method according to claim 9, wherein the neoplastic disease is a carcinoma originated from epithelial tissue selected from the group consisting of skin, lung, intestine, colon, breast, bladder, head and neck, esophagus, thyroid, kidney, liver, pancreas, bladder, penis, testes, prostate, vagina, cervix, anus, and any combination thereof.

11. The method according to claim 9, wherein the neoplastic disease is a squamous cell carcinoma (SCC), preferably a SCC of the skin, SCC of the head and neck, SCC of the oesophagus or SCC of the lung.

12. The method according to claim 1, wherein the subject is human.

13. The method according to claim 1, wherein the CAMK inhibitor is a CAMK2 inhibitor.

14. The method according to claim 1, wherein the genetic alteration which reduces or abolishes expression or function of FAT1 function is
one or more FAT1 loss-of-function mutations, or
a copy number variation (CNV) comprising the deletion of the FAT1 gene or a portion thereof.

15. The method according to claim 14, wherein the one or more FAT1 loss-of-function mutations are selected from the group consisting of a missense mutation, a nonsense mutation, a frameshift mutation, a splicing mutation, and a combination thereof.

16. The method according to claim 1, wherein the biological sample is a tumor biopsy or a liquid biopsy.
